(19) United States
(12) Patent Application Publication
(10) Pub. No.: US 2008/0299095 A1
(43) Pub. Date: Dec. 4, 2008

(54) Nup98-Hox Fusions for Expansion of Hemopoietic Stem Cells

(75) Inventors: R. Keith Humphries, Vancouver (CA); Guy Sauvageau, Montreal (CA)

Correspondence Address: MERCHANT & GOULD PC, P.O. BOX 2903, MINNEAPOLIS, MN, 55402-0903, US

(73) Assignees: BC Cancer Agency, Vancouver (CA); Universite de Montreal, Montreal (CA)

(21) Appl. No.: 11/632,091

(22) PCT Filed: May 16, 2005
(86) PCT No.: PCT/CA2005/000755

§ 371 Date: Jan. 9, 2007

Related U.S. Application Data
(60) Provisional application No. 60/586,917, filed on Jul. 9, 2004.

(51) Int. Cl. A61K 35/12; C12N 5/06; C12N 15/11; A61K 31/7088; A61P 31/00

(52) U.S. Cl. 424/93.21; 435/375; 424/93.7; 435/366; 536/23.5; 514/44

(57) ABSTRACT

Nucleic acid constructs encoding homeobox-nucleoporin fusions are disclosed, compositions comprising same, and methods which provide enhanced expansion of stem cells. In particular, an isolated nucleic acid construct encoding a NUP98-HOX fusion is provided, which when introduced into hemopoietic stem cells provides enhanced expansion of these cells. Methods of expanding stem cells in vivo or ex vivo and methods of treatment using the stem cells are also described.

### FIELD OF THE INVENTION

The invention relates to nucleic acid constructs, vectors and compositions comprising same, chimeric polypeptides, combinations of polypeptides, the use of same for enhancing expansion of stem cells, and therapeutic methods for using same.

### BACKGROUND OF THE INVENTION

Stem cell therapy and ex vivo gene therapy have become treatments of choice for a variety of inherited and malignant diseases. Expansion of hematopoietic stem cells (HSCs) has important clinical applications since it can contribute to an increase in the rate and magnitude of reconstitution of the stem cells following transplantation. The expansion of non-hematopoietic adult stem cells, including stem cells isolated from organs such as liver, pancreas, kidney, lung, etc., also has important clinical applications, particularly as an external source of cells for replenishing missing or damaged cells of tissues or organs.

Activation of cell intrinsic pathways has been studied as a possible means to increase expansion of HSCs. The activation of Notch-1 by Jagged-1 has been reported to expand multipotent colony-forming cells (CFC) and maintain HSCs with lympho-myeloid repopulation potential (Varnum Finney B et al, Nat. Med. 6: 1278-1281, 2003, Karnau, F. N. et al, J. Exp. Med. 192: 1365-1372, 2000). Cultures supplemented with soluble Sonic Hedgehog have also been reported to increase self-renewal of human HSCs (Bhardwaj, G. et al, Nat. Immunol. 2: 172-180, 2001). Retroviral expression of HOXB4 induced an increase in the number of transduced HSCs in a mouse bone marrow transplantation model (Sauvageau, G. et al, Genes Dev. 9:1753-1765, 1995; Thorsteinsdottir, U. et al, Blood, 94:2605-2612, 1999, Antonchuk, J. et al., Exp. Hematol, 29: 1125-1134, 2001), and promoted an increase in the rate and magnitude of hematopoietic reconstitution when compared to cells transduced with a control retroviral vector (Antonchuk, J. et al, supra). HOXB4 produced a similar effect on primitive human cells in immunocompromised NOD-SCID mice (Buske C et al, Blood 100: 862-868, 2002). HOXB4 has also been demonstrated to induce a rapid 40-fold ex vivo expansion of mouse HSCs without impairment in the normal production of mature cells (Antonchuk, J. et al, Cell 109: 39-45, 2002).

The citation of any reference herein is not an admission that such reference is available as prior art to the instant invention.

### SUMMARY OF THE INVENTION

The present invention relates to novel molecules, compositions, methods which provide or result in enhanced expansion of stem cells, and methods for using same (e.g. methods for research, development, and commercial purposes).

In an aspect the invention features an isolated nucleic acid construct comprising a homeobox polynucleotide and a sequence encoding a transcription activation domain (TAD) (e.g. nucleoporin) wherein the construct provides enhanced expansion of stem cells. In another aspect, a nucleic acid construct is provided comprising a homeobox polynucleotide fused to a sequence encoding a TAD (e.g. nucleoporin). An isolated nucleic acid construct of the invention may optionally comprise an exogenous gene, in particular an exogenous gene encoding a therapeutic. Further, a construct of the invention may comprise a nucleic acid sequence encoding a proliferation factor. Still further, a construct of the invention may comprise a nucleic acid sequence encoding an element that enhances or facilitates delivery of a polypeptide or polynucleotide to stem cells.

A sequence of a homeobox polynucleotide and a sequence encoding a TAD (e.g. nucleoporin) may be incorporated into an appropriate expression vector, i.e. a vector that contains the necessary regulatory elements for the transcription and translation of the inserted coding sequences. Accordingly, vectors adapted for transformation of a host cell (e.g. stem cell) may be constructed which comprise a sequence of a homeobox polynucleotide and a sequence encoding a TAD (e.g. nucleoporin) or a nucleic acid construct, and one or more regulatory elements necessary for transcription and translation, operably linked to the inserted coding sequences. The sequences may be under the control of the same or different regulatory sequences.

The vector can be used to prepare transformed host cells expressing an exogenous homeodomain polypeptide and an exogenous TAD polypeptide (e.g. nucleoporin), or a chimeric polypeptide comprising a homeodomain polypeptide and a TAD (e.g. nucleoporin). Therefore, the invention further provides host cells comprising or transformed with an exogenous homeobox polynucleotide and exogenous TAD (e.g. nucleoporin), a nucleic acid construct or vector of the invention.

The invention contemplates a modified stem cell comprising a homeobox polynucleotide and a sequence encoding a TAD (e.g. nucleoporin). In another aspect, a modified stem cell is provided comprising a nucleic acid construct of the invention, and preparations comprising stem cells modified to express a nucleic acid construct of the invention. The invention also contemplates a modified stem cell comprising a homeodomain polypeptide and a TAD (e.g. nucleoporin). A stem cell may be a long-term repopulating or pluripotent stem cell characterized by the ability to give rise to cells which retain the capability of self-renewal, and in some aspects to proliferate and differentiate into cells of all hematopoietic lineages. In an embodiment, the modified stem cell is a modified hematopoietic stem cell, in particular a human hematopoietic stem cell expressing the surface marker CD34.

In particular classes of embodiments of the invention, modified stem cells can comprise a sequence of a homeobox polynucleotide and a sequence encoding a TAD (e.g. nucleoporin), a nucleic acid construct, or a ABD-B-like HOX polynucleotide and one or more inducible regulatory element which when activated results in expression of the polynucleotides or nucleic acid construct thereby effecting expansion of the stem cells.

A stem cell of the invention may be modified by any means known in the art which results in delivery of a homeodomain polypeptide and TAD polypeptide (e.g. nucleoporin) into the cell, or stable integration and expression of a homeobox polynucleotide and a TAD, or a nucleic acid construct in the modified cell and its progeny.

In an aspect, the invention also provides a method for genetically modifying stem cells with a homeobox polynucleotide and a sequence encoding a TAD (e.g. nucleoporin) comprising obtaining stem cells to be genetically modified, providing the stem cells ex vivo with conditions for cell proliferation, and genetically modifying the stem cells with a homeobox polynucleotide and a sequence encoding a TAD (e.g. nucleoporin). In an aspect of the invention, the stem cells are modified with a nucleic acid comprising an exogenous homeobox polynucleotide and a separate nucleic acid molecule comprising a sequence encoding an exogenous TAD (e.g. nucleoporin). In another aspect, the stem cells are modified with a nucleic acid construct comprising a homeobox polynucleotide and a sequence encoding a TAD (e.g. nucleoporin).

The invention further provides a method for modifying stem cells comprising delivering a homeodomain polypeptide and a TAD polypeptide (e.g. nucleoporin) into the cells (e.g. by protein transduction).

The invention further provides a method for preparing a chimeric polypeptide comprising a homeodomain polypeptide and a TAD (e.g. nucleoporin) utilizing a purified and isolated nucleic acid construct of the invention. In an embodiment, a method is provided for preparing a chimeric polypeptide comprising a homeodomain polypeptide and a TAD (e.g. nucleoporin) comprising (a) transferring a vector comprising a homeobox polynucleotide and a sequence encoding a TAD polypeptide (e.g. nucleoporin) into a host cell; (b) selecting transformed host cells from untransformed host cells; (c) culturing a selected transformed host cell under conditions which allow expression of the chimeric polypeptide; and (d) isolating the chimeric polypeptide.

The invention still further provides a chimeric polypeptide comprising a homeodomain polypeptide and a TAD (e.g. nucleoporin), in particular a chimeric polypeptide produced by a method of the invention. In an aspect the invention provides a chimeric polypeptide comprising a fragment or part derived from a homeodomain polypeptide coupled to a TAD (e.g. nucleoporin).

The present invention also relates to a composition comprising a nucleic acid construct, a chimeric polypeptide of the invention, a homeobox polypeptide and a TAD (e.g. nucleoporin) or polynucleotides encoding same, and optionally a pharmaceutically acceptable carrier, excipient or diluent. A pharmaceutical composition may include a targeting agent to target cells to particular tissues or organs, an element that enhances or facilitates delivery of a polypeptide or polynucleotide to stem cells, and/or a proliferation agent.

The invention contemplates methods for enhancing expansion of stem cells. In aspects of the invention the methods utilize a nucleic acid construct, a homeobox polynucleotide and a sequence encoding a TAD (e.g. a nucleoporin polynucleotide), a chimeric polypeptide, a homeodomain polypeptide and a nucleoporin, or a composition of the invention or components thereof, to enhance expansion of stem cells.

In aspects of the constructs, vectors, host cells, compositions and methods of the invention, the homeobox polynucleotide is a HOX polynucleotide of the Abdominal B class (Abd-B-like HOX polynucleotides), for example, HOXA10 or HOXD13, or the homeodomain polypeptide is a member of the Abdominal B class, for example, HoxA10 or HoxD13. In other particular aspects, HOX polynucleotides of the Antennapedia class (Antp-like HOX polynucleotides), for example HOXB3 and HOXB4, or polypeptides encoded by these genes, are utilized.

According to an aspect of the invention, a method is provided for expanding a population of stem cells comprising modifying the stem cells with a (i) a sequence encoding a homeodomain polypeptide and a sequence encoding a transcription activation domain (TAD); (ii) a homeodomain polypeptide and a TAD; or (iii) an Abd-B-like HOX polynucleotide or a Abd-B-like polypeptide.

According to another aspect of the invention a method is provided for enhancing expansion of stem cells comprising delivering to the stem cells an effective amount of a nucleic acid construct, a homeobox polynucleotide and a sequence encoding a TAD (e.g. a nucleoporin polynucleotide), a chimeric polypeptide, a homeodomain polypeptide and a nucleoporin, or composition of the invention or components thereof, or a Abd-B-like HOX polynucleotide or polypeptide to provide enhanced expansion of the stem cells. In particular, a method of enhancing expansion of stem cells is provided comprising delivering to the stem cells an effective amount of (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-B-like HOX polynucleotide or a Abd-B-like polypeptide; to provide enhanced expansion of the stem cell

Expansion of stem cells may occur in vitro (i.e., prior to transplantation) and/or in vivo (i.e., enhanced regeneration of stem cells after transplantation or after delivery of a nucleic acid construct, composition, etc. to stem cells in a subject). In an embodiment, the stem cells are hematopoietic stem cells and the method is characterized by not altering the normal proportion of mature blood cells and/or the commitment to specific blood cell lineages obtained with nonmodified stem cells.

In aspects of methods of the invention described herein, a sequence encoding a homeodomain polypeptide and a sequence encoding a transcription activation domain may be fused to provide a nucleic acid construct, or a homeodomain polypeptide and a TAD may be fused to provide a chimeric polypeptide.

Expression of an exogenous homeobox polynucleotide and a sequence encoding a TAD (e.g. a nucleoporin), a nucleic acid construct, or an Abd-B-like HOX polynucleotide results in enhanced ability of modified stem cells to generate expanded populations of pluripotent stem cells (i.e. enhanced expansion of stem cells). In an embodiment, the stem cells are hematopoietic stem cells. In a more specific embodiment, the hematopoietic stem cells are human hematopoietic stem cells expressing the cell surface marker CD34.

Aspects of the invention provide methods for producing hematopoietic stem cells from embryonic stem cells, and methods for enhancing the output of hematopoietic stem cells from embryonic stem cells, in particular initiated or differentiated embryonic stem cells. In an embodiment, a method of producing hematopoietic stem cells from embryonic stem cells is provided comprising obtaining embryonic stem cells and modifying the stem cells with (i) a homeodomain polypeptide; (ii) a homeobox polynucleotide; (iii) a sequence encoding a homeodomain polypeptide and a sequence encoding a transcription activation domain (TAD); or (iv) a homeodomain polypeptide and a TAD so that the embryonic stem cells form hematopoietic stem cells. In another embodiment, a method for enhancing expansion or output of hematopoietic stem cells from embryonic stem is provided comprising modifying the embryonic stem cells with an effective amount of (i) a homeodomain polypeptide; (ii) a homeobox polynucleotide; (iii) a sequence encoding a homeodomain polypeptide and a sequence encoding a transcription activation domain (TAD); or (iv) a homeodomain polypeptide and a TAD, to generate expanded populations of hematopoietic stem cells

The invention features a method of expanding a population of hematopoietic stem cells by modifying the hematopoietic stem cells to express a sequence encoding a homeodomain polypeptide and a sequence encoding a TAD (e.g. nucleoporin), a homeodomain polypeptide and a TAD (e.g. nucleoporin), a nucleic acid construct, chimeric polypeptide, a composition of the invention or components thereof, or an Abd-B-like HOX polynucleotide or polypeptide. The methods can be performed in vitro or in vivo. The resulting expanded cell population can be characterized by the capacity to undergo substantial self-renewal and the ability to give rise to all hematopoietic cell lineages. The effect of a homeobox polynucleotide and a sequence encoding a TAD (e.g. nucleoporin), a nucleic acid construct, a homeodomain polypeptide and a TAD (e.g. nucleoporin), a chimeric polypeptide, a composition of the invention or component thereof, or an Abd-B-like polynucleotide or polypeptide on cell expansion results in no discernable effect on differentiation. The expanded cell population can give rise to mature blood cells in the same or similar proportions resulting from the expansion of nonmodified stem cells. Still further, when transplanted into a recipient subject, the expanded population of stem cells can substantially restore hematopoietic capability to a subject without the development of leukemia.

In an aspect, the invention provides a method for expanding hematopoietic stem cells and progenitor cells comprising (a) obtaining a sample comprising stem cells and enriching for stem cells by positive or negative selection; (b) modifying the enriched stem cells so that they comprise (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or, (iii) an Abd-B-like HOX polynucleotide or a Abd-B-like polypeptide; and (c) culturing and isolating increased numbers of stem cells and progenitor cells.

The invention provides a method for expanding hematopoietic stem cells and progenitor cells comprising (a) obtaining a sample comprising stem cells and enriching for stem cells by positive or negative selection, preferably enriching for CD34⁺ cells; (b) modifying the enriched stem cells so that they comprise a homeobox polynucleotide and a sequence encoding a TAD (e.g. a nucleoporin polynucleotide), a nucleic acid construct, vector, a composition of the invention or components thereof, a homeodomain polypeptide and a TAD (e.g. nucleoporin), or an Abd-B-like HOX polynucleotide or polypeptide; and (c) culturing and isolating increased numbers of stem cells and progenitor cells.

According to an aspect of the invention there is provided a method of ex vivo expanding stem cells comprising modifying the stem cells with a homeodomain polypeptide and a TAD (e.g. a nucleoporin polypeptide), nucleic acid construct, a homeobox polynucleotide and a sequence encoding a TAD (e.g. a nucleoporin polynucleotide), a composition or components thereof, chimeric polypeptide, or an Abd-B-like HOX polynucleotide or polypeptide of the invention, and culturing ex vivo to thereby expand the stem cells.

The invention also contemplates a method for enhancing the stability and/or potency of HOXB4 for enhancing expansion of stem cells comprising fusing HOXB4 to a sequence encoding a TAD (e.g. nucleoporin). In particular, a method is provided for enhancing the stability and/or potency of HOXB4 for enhancing expansion of stem cells comprising obtaining stem cells and modifying the stem cells with a HOXB4 polynucleotide fused to a sequence encoding a TAD wherein expansion of the stem cells is increased at least 10-fold, 20-fold, 50-fold, or 1000-fold relative to expansion of the stem cells with HOXB4 alone

The invention relates to an expanded cell preparation comprising modified stem cells obtained using a method of the invention and progeny thereof.

The invention also relates to an ex vivo expanded cell preparation obtained by modifying harvested stem cells with a nucleic acid construct, a homeobox polynucleotide and a sequence encoding a TAD (e.g. a nucleoporin polynucleotide), a composition of the invention or components thereof, a homeodomain polypeptide and a TAD (e.g. nucleoporin polypeptide), or an Abd-B-like HOX polynucleotide or Abd-B-like HOX polypeptide, and culturing the modified cells under proliferation conditions to thereby expand the harvested stem cells.

The ability of a homeobox polynucleotide and a sequence encoding a TAD (e.g. nucleoporin), a nucleic acid construct of the invention, Abd-B-like HOX polynucleotides and polypeptides, a homeodomain polypeptide and a TAD (e.g. nucleoporin), a chimeric polypeptide and composition of the invention and components thereof, to induce hematopoietic stem cell production and/or expand a population of long-term repopulating cells which retain the ability to give rise to all hematopoietic cell lineages in normal proportions and which is preferably not accompanied by development of leukemia is therapeutically useful.

Modified stem cells or cells in an expanded stem cell preparation of the invention comprising a homeodomain polypeptide and a TAD (e.g. nucleoporin polypeptide) or a Abd-B-like HOX polypeptide, or expressing a homeobox polynucleotide and a sequence encoding a TAD (e.g. nucleoporin polynucleotide), a nucleic acid construct of the invention, or a Abd-B-like HOX polynucleotide can be used in a variety of methods (e.g. transplantation or grafting) and they have numerous uses in the field of medicine. Preparations comprising modified stem cells or expanded stem cells preparations described herein may be used in both cell therapies (e.g. hematopoietic stem cell transplantation) and gene therapies aimed at alleviating conditions and/or diseases. The cell preparations can also be useful for a number of research, development, and commercial purposes.

The invention can be applied to the treatment of a condition and/or disease involving hematopoietic cells. According to an aspect of the invention a method of treatment is provided comprising administering a therapeutically effective amount of stem cells modified with one or more of a homeodomain polypeptide and a TAD (e.g. a nucleoporin polypeptide), a nucleic acid construct, a homeobox polynucleotide and a sequence encoding a TAD (e.g. a nucleoporin polynucleotide), a chimeric polypeptide, an Abd-B-like HOX polynucleotide or polypeptide, or composition of the invention or components thereof.

Thus, the invention contemplates a method of treating a patient with a condition and/or disease involving hematopoietic cells comprising transferring to a patient a therapeutically effective amount of a cell preparation comprising modified stem cells or expanded stem cells described herein.

In an aspect, the invention provides a method for treating a condition and/or disease in a subject in which reconstitution of stem cells is desirable comprising administering a therapeutically effective amount of stem cells modified with (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD and optionally an element that enhances or facilitates delivery of the sequences into stem cells; or (iii) an Abd-B-like HOX polynucleotide or a Abd-B-like polypeptide.

The invention has particular applications in preventing and/or treating conditions and/or diseases requiring reconstitution of the hematopoietic system. The invention relates to a method for ameliorating progression of, or obtaining a less severe stage of, a condition and/or disease in a subject suffering from a condition and/or disease requiring reconstitution of the hematopoietic system comprising administering a therapeutically effective amount of stem cells modified with a nucleic acid construct, a homeobox polynucleotide and a sequence encoding a TAD (e.g. a nucleoporin polynucleotide), a chimeric polypeptide, a homeodomain polypeptide and a TAD (e.g. nucleoporin), a composition of the invention or components thereof, or an Abd-B-like HOX polynucleotide or polypeptide. In aspects of the methods of the invention, the modified stem cells are administered to a subject and expanded in vivo. In other aspects, the modified stem cells are expanded in vitro and administered to a subject.

According to an aspect of the present invention there is provided a method of hematopoietic cell transplantation comprising obtaining hematopoietic stem cells to be transplanted from a donor; modifying the hematopoietic stem cells with a homeodomain polypeptide and a TAD (e.g. a nucleoporin), a nucleic acid construct, a homeobox polynucleotide and a sequence encoding a TAD (e.g. a nucleoporin polynucleotide), chimeric polypeptide, composition or components thereof, or an Abd-B-like HOX polynucleotide or polypeptide; culturing the hematopoietic stem cells under proliferation conditions to thereby expand the hematopoietic stem cells, and transplanting the hematopoietic stem cells to a patient. In an embodiment, the donor and patient is a single individual.

According to another aspect of the present invention there is provided a method of adoptive immunotherapy comprising obtaining hematopoietic stem cells from a patient; modifying the hematopoietic stem cells with a homeodomain polypeptide and a sequence encoding a TAD (e.g. a nucleoporin), a nucleic acid construct, a homeobox polynucleotide and a sequence encoding a TAD (e.g. nucleoporin polynucleotide), a chimeric polypeptide, a composition or components thereof, or a Abd-B-like HOX polynucleotide or polypeptide of the invention; culturing the hematopoietic stem cells under proliferation conditions to thereby expand the hematopoietic stem cells; and transplanting the hematopoietic stem cells to the patient. Adoptive immunotherapy of various malignancies and immunodeficiency, viral and genetic diseases, can enhance the required immune response or replace deficient functions.

The invention also features a therapeutic method for restoring hematopoietic capability to a human subject comprising recovering stem cells from a subject, modifying and expanding the stem cells in vitro as described herein, and returning the stem cells to the subject or a different subject, resulting in enhancement or restoration of hematopoietic capability to the subject.

In an aspect, the invention provides a therapeutic method for restoring hematopoietic capability to a mammalian subject, said method comprising the steps of: (a) removing hematopoietic stem cells from a mammalian subject; (b) modifying said stem cells to express or comprise a homeodomain polypeptide and a TAD (e.g. nucleoporin), a homeobox polynucleotide and a sequence encoding a TAD (e.g. nucleoporin polynucleotide), or a nucleic acid construct or chimeric polypeptide of the invention; (c) expanding said stem cells to form an expanded population of stem cells from said subject, and (d) returning said expanded cells to said subject, wherein hematopoietic capability is restored to said subject.

In an embodiment, the invention provides a method for preventing and/or treating leukemia in a patient comprising administering to the patient a therapeutically effective amount of stem cells modified with a homeodomain polypeptide and a nucleoporin, nucleic acid construct, a homeobox polynucleotide and a sequence encoding a TAD (e.g. nucleoporin polynucleotide), chimeric polypeptide, or composition of the invention or components thereof, or a Abd-B-like HOX polynucleotide or polypeptide.

A gene therapy aspect of the invention comprises removing hematopoietic stem cells from a subject, transducing the stem cells in vitro with an exogenous gene (e.g. therapeutic) and a nucleic acid construct, a sequence of a homeobox polynucleotide and a sequence encoding a nucleoporin, or an Abd-like HOX polynucleotide of the invention, and administering transduced stem cells to a subject wherein the stem cells having the capability of substantial self-renewal and ability to give rise to all hematopoietic cell lineages.

Another gene therapy aspect of the invention comprises removing hematopoietic stem cells from a subject, transducing the stem cells in vitro with an exogenous gene and a nucleic acid construct, a sequence of a homeobox polynucleotide and a sequence encoding a nucleoporin, or an Abd-like HOX polynucleotide, expanding the cells in vitro, and administering transduced stem cells to a subject wherein the stem cells having the capability of substantial self-renewal and ability to give rise to all hematopoietic cell lineages. These modified stem cells and their progeny will express the therapeutic in vivo.

The invention relates to methods for in vivo expanding stem cells in a subject in need thereof comprising administering to stem cells in the subject a therapeutically effective amount of a homeodomain polypeptide and nucleoporin, a nucleic acid construct, a homeobox polynucleotide and a sequence encoding a TAD (e.g. nucleoporin polynucleotide) and optionally an exogenous gene, a composition of the invention or components thereof, an Abd-B-like HOX polynucleotide or polypeptide, or a chimeric polypeptide of the invention, to thereby in vivo expand the stem cells in the subject. In an embodiment, stem cells of the subject are transduced in vivo with a homeodomain polypeptide and nucleoporin. In another embodiment, stem cells of the subject are transduced in vivo with a vector(s) comprising a nucleic acid construct, a homeobox polynucleotide and a sequence encoding a TAD (e.g. nucleoporin polynucleotide), an Abd-B-like HOX polynucleotide, and optionally an inducible regulatory element which is activated by an endogenous or exogenous factor (e.g. chemicals, chemo-attractants, particular ligands, and the like.

The invention provides use of a homeodomain polypeptide and a nucleoporin, nucleic acid construct, a homeobox polynucleotide and a sequence encoding a TAD (e.g. nucleoporin polynucleotide), chimeric polypeptide, or composition of the invention or components thereof, or a Abd-B-like HOX polynucleotide or polypeptide, in the preparation of a medicament for restoring hematopoietic capability to a mammalian subject. In addition the invention provides use of a therapeutically effective amount of stem cells modified with a homeodomain polypeptide and a nucleoporin, nucleic acid construct, a homeobox polynucleotide and a sequence encoding a TAD (e.g. nucleoporin polynucleotide), chimeric polypeptide, or composition of the invention or components thereof, or a Abd-B-like HOX polynucleotide or polypeptide, in the preparation of a medicament for treating a condition and/or disease in a subject in which reconstitution of stem cells is desirable; for restoring hematopoietic capability to a mammalian subject; for transplantation into a subject in need of such transplantation; and for adoptive immunotherapy. In aspects of the invention, the subject has a condition and/or disease involving hematopoietic cells, in particular leukemia.

Aspects of the invention contemplate use of a therapeutically effective amount of stem cells modified with a sequence encoding a exogenous gene (e.g. therapeutic) and a sequence encoding a homeodomain polypeptide and a sequence encoding a transcription activation domain (e.g. nucleoporin polynucleotide), a nucleic acid construct, or an Abd-B-like HOX polynucleotide in the preparation of a medicament for gene therapy.

The invention further provides a kit for carrying out the methods of the invention, and kits comprising components utilized in such methods.

These and other aspects, features, and advantages of the present invention should be apparent to those skilled in the art from the following detailed description.

### DETAILED DESCRIPTION OF THE INVENTION

In accordance with the present invention there may be employed conventional molecular biology, microbiology, recombinant DNA techniques, cell biology, and protein chemistry within the skill of the art. Such techniques are explained fully in the literature. See for example, Sambrook, Fritsch, & Maniatis, Molecular Cloning: A Laboratory Manual, Second Edition (1989) (Cold Spring Harbor Laboratory Press, Cold Spring Harbor, N.Y.); Current DNA Cloning: A Practical Approach, Volumes I and II (D. N. Glover ed. 1985); Oligonucleotide Synthesis (M. J. Gait ed. 1984); Transcription and Translation B. D. Hames & S. J. Higgins eds (1984); Animal Cell Culture R. I. Freshney, ed. (1986); Immobilized Cells and enzymes IRL Press, (1986); B. Perbal, A Practical Guide to Molecular Cloning (1984); Current Protocols in Molecular Biology (F. M. Ausubel et al. eds., Wiley & Sons); Current Protocols in Protein Science (J. E. Colligan et al. eds., Wiley & Sons); Current Protocols in Cell Biology (J. S. Bonifacino et al., Wiley & Sons); and, Current protocols in Immunology (J. E. Colligan et al. eds., Wiley & Sons.). Reagents, cloning vectors, and kits for genetic manipulation referred to herein can be obtained from commercial vendors including BioRad, Stratagene, Invitrogen, ClonTech, and Sigma-Aldrich Co. Cell culture methods are generally described in Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Tissue culture supplies and reagents can be obtained from commercial vendors including Gibco/BRL, Nalgene-Nunc International, Sigma Chemical Co., and ICN Biomedicals.

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions there of are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to “a cell” includes a plurality of cells, including mixtures thereof.

The terms “administering” or “administration” refers to the process by which cells, cell preparations, nucleic acid constructs, polypeptides, polynucleotide, chimeric polypeptides, or compositions of the invention, or components thereof, are delivered to a patient for treatment purposes. Cells, cell preparations etc. are administered in accordance with good medical practices taking into account the patient's clinical condition, the site and method of administration, dosage, patient age, sex, body weight, and other factors known to physicians. For example, cells, cell preparations, etc. may be administered a number of ways including but not limited to parenteral (e.g. intravenous and intra-arterial as well as other appropriate parenteral routes), subcutaneous, or transdermal. The terms “transplanting” “transplantation”, “grafting” and “graft” are also used herein to describe a method of administration in which cells, modified cells, and cell preparations are delivered to the site within the patient where the cells are intended to exhibit a favorable effect, such as repairing damage to a patient's tissues, treating a disease, injury or trauma, or genetic damage or environmental insult to an organ or tissue caused by, for example an accident or other activity. Cells, modified cells, and cell preparations may also be delivered in a remote area of the body by any mode of administration relying on cellular migration to the appropriate area in the body to effect transplantation.

An “analog” refers to a polypeptide wherein one or more amino acid residues of a parent or native polypeptide have been substituted by another amino acid residue, one or more amino acid residues of a parent or native polypeptide have been inverted, one or more amino acid residues of the parent polypeptide have been deleted, and/or one or more amino acid residues have been added to the parent polypeptide. Such an addition, substitution, deletion, and/or inversion may be at either of the N-terminal or C-terminal end or within the parent or native polypeptide, or a combination thereof. Mutations may be introduced into a polypeptide by standard methods, such as site-directed mutagenesis and PCR-mediated mutagenesis. Conservative substitutions can be made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed.

An “antibody” includes but is not limited to a monoclonal or polyclonal antibody, immunologically active fragments (e.g. a Fab, (Fab)₂ fragment, or Fab expression library fragments and epitope-binding fragments thereof), an antibody heavy chain, an antibody light chain, a genetically engineered single chain Fv molecule (Ladner et al, U.S. Pat. No. 4,946,778), humanized antibody, or a chimeric antibody, for example, an antibody which contains the binding specificity of a murine antibody, but in which the remaining portions are of human origin. Antibodies can be prepared using methods known to those skilled in the art.

A “cationic delivery vehicle” refers to a vehicle that is adapted to fuse with a cell membrane to effect intracellular delivery of an associated polypeptide. A vehicle may comprise a cationic lipid, cationic liposome, a lipoplex comprising a cationic lipid and nucleic acid, and an anionic polymer in association with a cationic lipid wherein the anionic polymer (e.g. biopolymer such as a nucleic acid or a synthetic polymer) comprises an anionic group coupled to the polypeptide to be delivered.

A cationic delivery vehicle may be linked directly or indirectly (through a linker) to an associated polypeptide. For example, a polypeptide may be linked to a polynucleotide and the polynucleotide may be associated with a cationic lipid such as through a peptide nucleic acid (PNA) linker or a linker that is linked to a cationic lipid. In embodiments of the invention at least one of the protein-linker and linker-cationic lipid associations is covalent. In other embodiments, at least one of the protein-linker and linker-cationic lipid associations is ionic. Examples of cationic delivery vehicles are described in PCT Published Application No. WO 03095641 (Application WO 2003US0013873).

The term “chimeric” describes and relates to polypeptides wherein two individual or distinct polypeptides or portions thereof are fused to form a single amino acid chain. Such fusion may arise from the expression of a single continuous coding sequence formed by recombinant techniques. Chimeric polypeptides include contiguous polypeptides comprising a homeodomain polypeptide or portion thereof covalently linked via an amide bond to one or more amino acid sequences which define a TAD (e.g. nucleoporin) or portion thereof. A chimeric polypeptide of the invention can comprise one or more regions of a homeodomain polypeptide and/or a TAD (e.g. nucleoporin) which can be contiguous in the chimeric polypeptide or scattered throughout the chimeric polypeptide. A chimeric polypeptide can also have sequences derived from different species and it can comprise additional sequences separating or flanking the homeodomain polypeptide and nucleoporin. A chimeric polypeptide may also comprise additional polypeptide or peptide sequences including a sequence of an element that enhances or facilitates delivery of a polypeptide or polynucleotide to stem cells.

“Condition(s) and/or “disease(s)” include conditions or diseases requiring partial, substantial, or complete reconstitution of stem cells or that involve a dysfunction of stem cells. The terms include conditions or diseases requiring partial, substantial, or complete reconstitution of the hematopoietic system, in whole or in part, in particular neoplastic, infectious or genetic diseases. A condition and/or disease includes an acute or chronic hematopoietic dysfunction such as an inherited deficiency of the erythroid, granulocytic, macrophage, megakaryocyte, or lymphoid lineage, inadequate hematopoietic capacity causing anemia or immune deficiency, or hematopoietic toxicity. Examples of conditions and/or diseases are leukemia (e.g. acute myelogenous leukemia, chronic myelogenous leukemia), lymphomas (e.g. non-Hodgkin's lymphoma), neuroblastoma, testicular cancer, multiple myeloma, melanomas, breast cancer, solid tumors that have a stem cell etiology, or other cancers in which therapy results in the depletion of hematopoietic cells, HIV, autoimmune diseases such as lupus, exposure to radiation, genetic diseases including but not limited to β-thalassemia (Mediterranean anemia), sickle cell anemia, aplastic anemia, myelodysplastic syndrome, ADA deficiency, recombinase deficiency, recombinase regulatory gene deficiency and the like, and diseases relating to a deficiency of secretory proteins such as hormones, enzymes, cytokines, growth factors and the like.

Conditions and/or diseases that are also contemplated herein include those that require partial, substantial, or complete replacement or replenishment of non-hematopoietic cells, tissues, or organs. For example, expansion of neural stem cells or oligodendrocyte progenitors may be useful in treating neurological diseases, in particular, myelin disorders. In addition, ex vivo and in vivo expansion of stem cells can be used for example in mesenchymal tissue regeneration or repair, skin regeneration, hepatic regeneration, muscle regeneration, and bone growth in osteoporosis.

A “derivative” refers to a polypeptide in which one or more of the amino acid residues of a parent or native polypeptide have been chemically modified. A chemical modification includes adding chemical moieties, creating new bonds, and removing chemical moieties. A polypeptide may be chemically modified, for example, by alkylation, acylation, glycosylation, pegylation, ester formation, deamidation, or amide formation.

“Detectable substances” include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags).

“Enhanced expansion of stem cells” refers to an increase in the number of stem cells and progenitor cells compared to a control. In particular, the control is the amount of stem cell expansion obtained with expression of HOXB4 alone. The increase in the number of cells can be at least about 2-fold to 20-fold, 2-fold to 50-fold, 2-fold to 100-fold, 10-fold to 20-fold, 10-fold to 50-fold, 10-fold to 100-fold, 10-fold to 200 fold, 10-fold to 300-fold, 10-fold to 400-fold, 10-fold to 500-fold, 10-fold to 600 fold, 10-fold-700 fold, 10-fold to 800-fold, 10-fold to 900-fold, 10-fold to 1000-fold, 10-fold to 1100-fold, 10-fold to 1200-fold, 10-fold to 1300-fold, 10-fold to 1400-fold, or 10-fold to 1500-fold increase relative to the number of stem cells and progenitor cells that are present in a parallel control culture of cells that are not modified with a nucleic acid construct, a homeobox polynucleotide and a sequence encoding a TAD (e.g. nucleoporin polynucleotide), vector, a homeodomain polypeptide and a nucleoporin, a chimeric polypeptide, composition, or Abd-B-like HOX polynucleotide or polypeptide, or a parallel control culture of cells that are modified with HOXB4 alone. More preferably, the increase in the number of cells can be at least a 10- to 100-fold expansion or at least a 50-fold, 100-fold, 500-fold, or 1000-fold expansion relative to the number of stem cells and progenitor cells that are present in a parallel control culture of cells that are not modified as described herein. The term “expand” or “expansion” contemplates the process of proliferation of stem cells substantially devoid of cell differentiation. Cells that undergo expansion maintain their renewal properties. An enhanced expansion of stem cells can be statistically significant.

“Exogenous gene” refers to a gene expressing a gene product including but not limited to proteins, peptides, glycoproteins, lipoproteins, and products produced by way of gene replacement to defective organs such as insulin, amylase, protease, lipase, phospholipase, and elastase, gene products produced by the liver including blood clotting factors, UDP glucuronyl transferase, ornthine transcarbanoylase, cytochrome p450 enzymes, adenosine deaminase, gene products produced by the thymus such as serum thymic factor, thymic humoral factor, thymoprotein, and thymosin, and gene products produced by the digestive tract including gastrin, secretin, cholecystokinin, somatostatin, serotonin, and substance P. An exogenous gene does not include a homeobox polynucleotide or a sequence encoding a TAD (e.g. nucleoporin polynucleotide). In an aspect of the invention the exogenous gene encodes a therapeutic.

“Gene therapy” refers to the transfer and stable insertion of new genetic information into cells for the therapeutic treatment of conditions and/or diseases described herein. An exogenous gene is transferred into a cell that proliferates to introduce the transferred gene throughout the cell population. Therefore, stem cells may be the target of gene transfer, since they will produce various lineages that will potentially express the exogenous gene. In aspects of the invention, an exogenous gene encodes a therapeutic.

There are two approaches to gene therapy: (i) ex vivo or cellular gene therapy; and (ii) in vivo gene therapy. In ex vivo gene therapy cells are removed from a patient, and while being cultured are treated in vitro. An exogenous gene is introduced into the cells via an appropriate delivery vehicle/method (transfection, transduction, homologous recombination, etc.) and regulatory elements as required, and the modified cells are expanded in culture and returned to the patient. The genetically re-implanted cells express the transfected exogenous gene in situ. In in vivo gene therapy an exogenous gene is introduced into tissues and cells in subjects, for example, by systemic administration or direct injection into sites in situ. General references describing using stem cells as vehicles for gene therapy and clinical applications include Stem Cell Biology and Gene Therapy by P. J. Quesenberry et al., (eds), John Wiley & Sons, 1998; and Blood Cell Biochemistry: Hematopoiesis and Gene Therapy (Blood Cell Biochemistry, Vol. 8) by L. J. Fairbairn & N. G. Testa (eds)., Kluwer Academic Publishers, 1999.

As used herein, “hematopoietic cells” refers to cells from the hematopoiesis pathway or cells that are related to the production of blood cells, including cells of the lymphoid, myeloid and erythroid lineages. The cells can express some of the phenotypic markers or morphological features characteristic of the hematopoietic lineage. Hematopoietic cells include hematopoietic progenitors, committed replication-competent or colony forming cells, and fully differentiated cells. A hematopoietic progenitor is a cell that is capable of generating fully differentiated hematopoietic cells and is capable of self-renewal.

Exemplary hematopoietic cells include early lineage cells of the mesenchymal, myeloid, lymphoid and erythroid lineages, bone marrow cells, blood cells, umbilical cord blood cells, stromal cells, and other hematopoietic cells that are known to those of ordinary skill in the art. The hematopoietic cells may be obtained from fresh blood, reconstituted cryopreserved blood, or fresh or reconstituted fractions thereof. The hematopoietic cells are preferably mammalian cells, more preferably, the cells are primate, pig, rabbit, dog, or rodent (e.g. rat or mouse) in origin. Most preferably, the cells are human in origin. The hematopoietic cells may be obtained from a fetus, a child, an adolescent, or an adult. The hematopoietic cells may also be derived from embryonic stem cells, in particular initiated or differentiated embryonic stem cells.

A “homeodomain polypeptide” refers to a polypeptide comprising a homeodomain which comprises a highly-conserved structural motif of about 60 amino acids. The tertiary structure of the homeodomain, as first determined by solution NMR studies of the Antennapedia homeodomain from Drosophila, consists of three alpha-helices folded into a compact, globular structure, with a flexible N-terminal arm (Kissinger C, et al, (1990) Cell 63:579-590; Wolberger, C. et al (1991) Cell 67:517-528; Wilson et al, (1995) Cell 82:709-719; Billeter, M. et al, (1993) J. Mol. Biol 234:1084-1093; Hirsch J. A. and Aggarwal A. K., (1995) EMBO J. 14:6280-6291; Li, T et al, (1995) Science 270:262-269). The structure comprises second and third helices of the compact three-helix domain that are structurally similar to a helix-turn-helix motif of the prokaryotic repressors.

A homeodomain polypeptide includes native-sequence or synthetic polypeptides, fragments or portions thereof, analogs (e.g. muteins), derivatives, isoforms, variants, polypeptides with sequence identity, peptidomimetics, polypeptides encoded by a homeobox polynucleotide, and pharmaceutically acceptable salts thereof.

Homeodomain polypeptides can be found on the Homeodomain Resource database of the National Human Genome Research Institute, National Institutes of Health at http://research.nhgri.nih.gov/homeodomain. [See also Lawrence, H. J., Sauvageau, G., Largman, C., and Humphries, R. K. (2001). Homeobox gene networks and the regulation of hematopoiesis. In Hematopoiesis: A Developmental Approach, L. I. Zon, ed. (Oxford, Oxford University Press), pp. 402-416.]

In aspects of the invention the homeodomain polypeptide is a member of the Antennapedia subfamily or Abdominal B subfamily. In a particular aspect, the homeodomain polypeptide is an Abd-B like Hox polypeptide. More particularly the homeodomain polypeptide is homeobox protein HoxA9, homeobox protein Hox A10, or homeobox protein HoxD13. In another particular aspect, the homeodomain polypeptide is an Antennapedia-like Hox polypeptide belonging to paralog groups 9 through 13 of HOX A, B, C, or D clusters (see below). The sequences of Antennapedia proteins contain a conserved hexapeptide 5-16 residues upstream of the homeobox. More particularly the homeodomain polypeptide is homeobox protein HoxB3 or homeobox protein HoxB4. See Table 1 and the Sequence Listing for sequences of representative homeodomain polypeptides.

A homeodomain polypeptide for applications of the present invention may comprise a fragment or portion, in particular a fragment or portion comprising a homeodomain alone or with additional flanking sequence such as a PBX Interacting Motif (PIM). In some aspects of the invention, a homeodomain polypeptide comprises a fragment including the DNA binding domain but excluding the PIM. In particular aspects of the invention the homeodomain polypeptide comprises or consists essentially of a homeodomain.

A “homeobox polynucleotide” comprises a homeobox that is about 180 base pairs long encoding a protein domain (the homeodomain) which can bind DNA and switch on cascades of genes. In an aspect, a homeobox polynucleotide encodes a homeodomain polypeptide. A particular subgroup of homeobox polynucleotides are the HOX genes, which are found in a special gene cluster, the HOX cluster. The term includes the four mammalian homeobox containing gene clusters (HOX-clusters A, B, C, and D) that are a highly conserved group of genes evolutionary related to the Drosophila Antennapedia- and Bithorax-complexes. HOX genes similarly located in the different clusters are subgrouped by virtue of their homology to different Drosophila HOM-C genes. Members belonging to the same subfamily (paralogs) have similar anterior limits and patterns of expression. There are 13 subfamilies including but not limited to: Abd-B, Abd-A, Ubx, Antp, Scr, Dfd, Zen, pb, and lab. [See Burglin, T. R. (1996) Homeodomain Proteins. In Meyers, R. A. (ed.), Encyclopedia of Molecular Biology and Molecular Medicine, Vol 3., VCH Verlagsgesellschaft mbH, Weinheim, pp. 55-76 for a review of homeobox polynucleotides.]

In embodiments of the invention the homeobox polynucleotide is an Abd-B-like HOX polynucleotide or Ant-like HOX polynucleotide. In particular the homeobox polynucleotide is HOXB4, HOXA9, HOXA10, HOXD13, HOXB3, more particularly HOXA10, HOXA9, or HOXD13.

See Table 1 and the Sequence Listing for sequences of representative homeobox polynucleotide sequences.

Polynucleotides or nucleic acids referenced herein include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

Polynucleotides also include sequences that differ from a native sequence due to degeneracy in the genetic code. As one example, DNA sequence polymorphisms within the nucleotide sequence of a homeobox polynucleotide may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide. Polynucleotides also include truncated nucleic acids or nucleic acid fragments and variant forms of the nucleic acids.

Further, polynucleotides include nucleic acids that hybridize under stringent conditions, preferably high stringency conditions to a homeobox polynucleotide. Appropriate stringency conditions which promote DNA hybridization are known to those skilled in the art, or can be found in Current Protocols in Molecular Biology, John Wiley & Sons, N0. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

A polynucleotide or nucleic acid described herein includes DNA and RNA (e.g. mRNA) and can be either double stranded or single stranded. A polynucleotide or nucleic acid may, but need not, include additional coding or non-coding sequences, or it may, but need not, be linked to other molecules. A polynucleotide or nucleic acid for use in the methods of the invention may be of any length suitable for a particular method.

A homeodomain polypeptide or a homeobox polynucleotide can be selected for use in the present invention that has one or more of the following characteristics: (i) it provides enhanced proliferative potential of stem cells to generate a population of pluripotent or long-term repopulating stem cells which give rise to all hematopoietic cell lineages; (ii) it is transplantable; (iii) it is able to restore hematopoietic capability to a mammal upon transplantation; and (iii) it does not result in leukemogenesis in the transplanted subject.

“Host cells” include a wide variety of prokaryotic and eukaryotic host cells. For example, constructs and polynucleotides or nucleic acids described herein may be expressed in bacterial cells such as E. coli, Bacillus, or Streptomyces, insect cells (using baculovirus), yeast cells, or mammalian cells. Other suitable host cells can be found in Goeddel, Gene Expression Technology: Methods in Enzymology 185, Academic Press, San Diego, Calif. (1991). A host cell may also be chosen which modulates the expression of an inserted nucleotide sequence, or modifies (e.g. glycosylation or phosphorylation) and processes (e.g., cleaves) the polypeptide in a desired fashion. Host systems or cell lines may be selected which have specific and characteristic mechanisms for post-translational processing and modification of proteins. In embodiments of the invention a host cell is a stem cell, in particular a hematopoietic stem cell or embryonic stem cell.

“Inducible regulatory element” refers to a regulatory element that induces expression of a gene to which it is operably linked in response to particular stimuli such as chemicals, chemo-attractants, particular ligands, and the like. An inducible regulatory element (e.g., an inducible promoter) permits modulation of the production of a gene product in a cell. Examples of suitable inducible regulatory systems for use in eukaryotic cells include hormone-regulated elements (e.g., see Mader, S, and White, J. H. (1993) Proc. Natl. Acad. Sci. USA 90: 5603-5607), synthetic ligand-regulated elements (see, e.g., Spencer, D. M. et al 1993) Science 262: 1019-1024) and ionizing radiation-regulated elements (e.g., see Manome, Y. Et al. (1993) Biochemistry 32:10607-10613; Datta, R. et al. (1992) Proc. Natl. Acad. Sci. USA 89: 1014-10153). Additional tissue-specific or inducible regulatory systems, which may be developed, can also be used in accordance with the invention.

“Isolated” or “purified” refers to altered “by the hand of man” from the natural state i.e. anything that occurs in nature is defined as isolated when it has been removed from its original environment, or both. For example, the term “isolated” when applied to a polynucleotide refers to a nucleic acid substantially free of cellular material or culture medium when produced by recombinant DNA techniques, or chemical reactants, or other chemicals when chemically synthesized. An “isolated” polynucleotide may also be free of sequences which naturally flank the nucleic acid (i.e., sequences located at the 5′ and 3′ ends of the nucleic acid molecule) from which the nucleic acid is derived.

The terms “long-term repopulating stem cell” and “CRU” are used interchangeably to mean stem cells capable of self-renewal and of giving rise to all hematopoietic cell lineages.

“Modified stem cell” refers to a stem cell into which exogenous genetic material (e.g. a nucleic acid construct of the invention, or a homeobox polypeptide and a nucleoporin) has been operatively incorporated into its genome, or into which a chimeric polypeptide or exogenous polypeptides (e.g. homeodomain polypeptide and nucleoporin) or compositions disclosed herein have been introduced. The modified stem cell is characterized by an enhanced ability to undergo self-renewal as compared to an unmodified stem cell. In an aspect a modified stem cell of the invention has a stably incorporated nucleic acid construct or a homeobox polynucleotide and a sequence encoding a TAD (e.g. nucleoporin polynucleotide), expression of which results in a 2-fold to 20-fold, 2-fold to 50-fold, 2-fold to 100-fold, 10-fold to 20-fold, 10-fold to 50-fold, or 10-fold to 100-fold, 10-fold to 200-fold, 10-fold to 300-fold, 10-fold to 400-fold, 10-fold to 500-fold, 10-fold to 600-fold, 10-fold-700 fold, 10-fold to 800-fold, 10-fold to 900-fold, 10-fold to 1000-fold, 10-fold to 1100-fold, 10-fold to 1200-fold, 10-fold to 1300-fold, 10-fold to 1400-fold, or 10-fold to 1500-fold expansion of a pluripotent stem cell population relative to expansion of stem cells modified with HOXB4 alone. More preferably, the stably incorporated construct or polynucleotides result in a 10- to 100-fold expansion or at least a 50-fold, 100-fold, 500-fold, or 1000-fold expansion of a pluripotent stem cell population relative to expansion of stem cells modified with HOXB4 alone. In another aspect a modified stem cell of the invention comprises an exogenous homeodomain polypeptide and TAD (e.g. nucleoporin) which results in a 2-fold to 20-fold, 2-fold to 50-fold, 2-fold to 100-fold, 10-fold to 20-fold, 10-fold to 50-fold, 10-fold to 100-fold, 10-fold to 200-fold, 10-fold to 300-fold, 10-fold to 400-fold, 10-fold to 500-fold, 10-fold to 600-fold, 10-fold-700 fold, 10-fold to 800-fold, 10-fold to 900-fold, 10-fold to 1000-fold, 10-fold to 1100-fold, 10-fold to 1200-fold, 10-fold to 1300-fold, 10-fold to 1400-fold, or 10-fold to 1500-fold expansion of a pluripotent stem cell population relative to expansion of stem cells modified with HOXB4 alone. More preferably, the modified stem cell comprising an exogenous homeodomain polypeptide and a TAD (e.g. nucleoporin) results in a 10- to 100-fold expansion or at least a 50-fold, 100-fold, 500-fold, or 1000-fold expansion of a pluripotent stem cell population relative to expansion of stem cells modified with HOXB4 alone.

A “native-sequence polypeptide” or “a native polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

A “nucleoporin” refers to a component of the nuclear pore complex (for reviews see Rout and Wente, 1994; Bastos et al., 1995). The term in particular refers to a member of the nucleoporin family containing the highly repeated peptide motifs, FXFG and FG. More particularly the term refers to Nup358, Nup214, Nup98, and Nup153, or portions thereof. [See SEQ ID NOs.: 2, 26, 28, 30, 32 and 34] Most particularly the term refers to Nup98, or portions thereof, in particular portion comprising the repeat peptide motif FG or FGF. (See B. M. A. Fontura et al, J. Cell Biol. 144(6): 1097, 1999 and references referred therein). A nucleoporin includes native-sequence or synthetic polypeptides, fragments, analogs (e.g. muteins), derivatives, isoforms, variants, polypeptides with sequence identity, peptidomimetics, and pharmaceutically acceptable salts thereof. In particular aspects of the invention, a fragment of a Nup98 polypeptide comprising a FGF repeat region is utilized.

A “nucleic acid encoding a nucleoporin” or “nucleoporin polynucleotide” refers to a sequence encoding a nucleoporin. A nucleoporin polynucleotide includes but is not limited to NUP358, NUP214, NUP98, and NUP153, or fragments thereof. [See SEQ ID NOs.: 1, 27, 29, 31, 33, and 34.] In a particular embodiment, the nucleoporin polynucleotide is NUP98 or a fragment thereof.

As defined herein “operably linked” means that a polynucleotide and a regulatory element are situated within a nucleic acid construct or cell in such a way that the polypeptide product is expressed by a host cell which has been transformed (transfected) with the ligated polynucleotide/regulatory element sequence.

A “peptidomimetic” refers to a synthetic chemical compound that has substantially the same structural and/or functional characteristics of a polypeptide described herein. A peptidomimetic can be composed entirely of synthetic, non-natural analogues of amino acids, or, is a chimeric molecule of partly natural peptide amino acids and partly non-natural analogs of amino acids. A polypeptide can be characterized as a peptidomimetic when all or some of its residues are joined by chemical means other than natural peptide bonds (see, e.g., Spatola (1983) in Chemistry and Biochemistry of Amino Acids, Peptides and Proteins, Vol. 7, pp 267-357, “Peptide Backbone Modifications,” Marcell Dekker, N.Y.). Peptidomimetics also include peptoids, oligopeptoids (Simon et al (1972) Proc. Natl. Acad, Sci USA 89:9367); and peptide libraries containing peptides of a designed length representing all possible sequences of amino acids corresponding to a motif or peptide.

“Percent identity” of two amino acid sequences, or of two nucleic acid sequences identified herein is defined as the percentage of amino acid residues or nucleotides in a candidate sequence that are identical with the amino acid residues in a polypeptide or polynucleotide sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST programs are publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95%, 98%, or 99% amino acid sequence identity to the sequences identified in any of SEQ ID NOs. 1 through 36. Such variants include, for example, polypeptides wherein one or more amino acid residues are added to, or deleted from, the full-length or mature sequences of SEQ ID NOs: 1 through 36 including variants from other species, but excludes a native-sequence polypeptide.

“Proliferation factor” refers to a protein, peptide, phosphopeptide, glycoprotein, lipoprotein, antibody, polynucleotide, ribozyme, carbohydrate, small molecule which when introduced in a stem cell results in enhanced proliferative potential of stem cells to generate a population of pluripotent or long-term repopulating stem cells. A proliferation factor does not include a homeodomain polypeptide or a TAD (e.g. nucleoporin). Examples of proliferation factors include activators of Notch-1 (e.g. Jagged-1, see Varnum-Finney et al, Nat. Med 6:1278-1281, 2000; Karanu, F N et al, J. Exp. Med. 192:1365-1372, 2000), Sonic-2 Hedgehog (see Bhardwaj G et al, Nat. Immunol. 2:172-180, 2001), blockers which reduce expression levels of at least one gene normally limiting HOX-induced expansion of stem cells [e.g. antisense, antibody, SiRNA, a peptide, chemical compound such as antisense DNA to PBx1, PBx2, PBx3 and PBx4; see PCT Publication No. WO 04033672 (Application No. WO 2003CA/0001539), fibroblast growth factor-1, thrombopoietin, stem cell factor (see for example, U.S. Pat. No. 6,852,313], telomerase reverse transcriptase, activated STAT3 [see for example, PCT Publication No. WO 03068952 (Application Serial No. WO20031B0000515)], and activators of the wnt signalling pathway including β-catenin, Wnt10B, Frizzled 1, Frizzled 5, and histone deacetylase inhibitors (e.g. valproic acid).

“Regulatory element” refers to a genetic element or elements having a regulatory role in gene expression, for example, promoters or enhancers. A regulatory element can be a constitutive or an inducible transcriptional regulatory region (i.e. inducible regulatory element). Suitable regulatory elements may be obtained from a variety of sources, including bacterial, fungal, viral, mammalian, or insect genes. (For example, seethe regulatory sequences described in Goeddel, GeneExpression Technology: Methods in Enzymology 185, Academic Press, San Diego, Calif. (1990)). Other sequences, such as an origin of replication, additional DNA restriction sites, enhancers, and sequences conferring inducibility of transcription may also be incorporated into the expression vector.

“Stem cell” refers to cells that are capable under appropriate conditions of producing progeny of several different cell types that are derivatives of all of the three germinal layers (endoderm, mesoderm, and ectoderm). In particular, a stem cell refers to a pluripotent cell capable of self-regeneration when provided to a subject in vivo, and gives rise to lineage restricted progenitors, which further differentiate and expand into specific lineages. Stem cells include a population of cells having all of the long-term engrafting potential in vivo.

In some aspects the term includes hematopoietic cells and may include stem cells of other origins such as stem cells from liver, pancreas, epithelium, neuron and bone marrow mesenchymal stem cells. In particular aspects, the term “stem cells” refers to mammalian hematopoietic stem cells; more particularly, the stem cells are human hematopoietic stem cells. An enriched stem cell population is preferably used in the present invention. For example, an enriched hematopoietic stem cell population can comprise a population of cells which have been selected by expression of the CD34 surface marker, lack of expression of lineage specific markers (Lin⁻), and/or which demonstrate selective enrichment of primitive pluripotent cells by functional assays, such as the in vitro initiating cell assay (LTCIC) (Sutherland et al. (1990) Proc. Natl. Acad. Sci. 87:3584-3588) or the in vivo CRU assay.

In aspects of the invention, the stem cells are capable of differentiating into non-hematopoietic tissues including without limitation liver, heart, kidney, or nervous tissues.

Stem cells may be isolated from any known source of stem cells, and can be obtained from any tissue of any multicellular organism. The term includes cells obtained from primary tissue that are pluripotent and established cell lines of stem cells. In particular, stem cells may be isolated from embryonic tissues, fetal tissues, bone marrow, both adult and fetal, mobilized peripheral blood and umbilical cord blood. In an aspect, bone marrow cells are obtained from a source of bone marrow, including ilium (e.g. from the hip bone via the iliac crest), tibia, femora, spine, or other bone cavities. Other sources of stem cells include but are not limited to embryonic yolk sac, fetal liver, fetal spleen, fetal para-aortic region (AGM region), and stem cells of other cell types, such as skin and gut epithelial cells, hepatocytes, mesenchymal cells, stromal cells, and neuronal cells.

Stem cells can also be derived from embryonic cells of various types, in particular, embryonic stem cells and more particularly initiated or differentiated embryonic stem cells. An “initiated” embryonic stem cell is an embryonic stem cell that has been initiated into differentiation in a non-specific way. Embryonic stem cells can be differentiated in a non-specific way using methods described herein including culturing the cells in a medium that supports differentiation, withdrawing factors that inhibit differentiation, including retinoic acid or dimethyl sulfoxide in the culture medium, or by forming primitive ectoderm-like cells (Rathjen et al., J. Cell Sci. 112:601, 1999). “Differentiated” in respect to a cell refers to a cell that has progressed further down the developmental pathway than the cell it is being compared with. An embryonic stem cell can differentiate to lineage-restricted precursor cells including a multipotent hematopoietic progenitor cell that is capable of forming cells of each of the erythroid, granulocyte, monocyte, megakaryocyte and lymphoid lines. These hematopoietic progenitor cells are capable of differentiating into self-renewing cells that are committed to form cells of only one of the four hematopoietic lines. The self-renewing cells can differentiate into terminally differentiated cells such as erythrocytes, monocytes, macrophages, neutrophils, eosinophils, basophils, platelets, and lymphocytes. Stem cells also include embryonic germ (EG) cells (Shamblott et al., Proc. Natl. Acad. Sci. USA 95:13726, 1998).

The term “subject” or “patient” refers to an animal including a warm-blooded animal such as a mammal, which is afflicted with or suspected of having or being pre-disposed to a condition and/or disease described herein. Mammal includes without limitation any members of the Mammalia. In general, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. The methods herein for use on subjects/patients contemplate prophylactic as well as curative use. Typical subjects for treatment include persons susceptible to, suffering from or that have suffered a condition and/or disease requiring reconstitution of stem cells in particular reconstitution of the hematopoietic system.

A “therapeutic” is used in a generic sense and includes treating agents, prophylactic agents, and replacement agents. Therapeutics can directly exert a therapeutic effect or they can have a less direct effect (e.g. a polypeptide that elicits an immune response). Examples include without limitation a peptide, polypeptide, oligonucleotide, polynucleotide, an enzyme, an enzyme inhibitor, an antigen, an antibody, a hormone, a factor involved in cell intrinsic pathways, an interferon, a cytokine, a chemokine, atrophic protein, a growth factor, or a tumor toxic protein.

A “therapeutically effective amount” refers to the amount or dose of stem cells, expanded stem cell preparation, a composition, a chimeric polypeptide, a nucleic acid construct, a homeodomain polypeptide and a nucleoporin, a homeobox polynucleotide and a sequence encoding a TAD (e.g. nucleoporin polynucleotide), or a Abd-B like Hox polynucleotide or polypeptide that will lead to one or more desired therapeutic effects. A therapeutically effective amount can vary according to factors such as the disease state, age, sex, and weight of the individual, and the ability of the substance to elicit a desired response in the individual. Dosage regimens may be adjusted to provide the optimum therapeutic response (e.g. sustained beneficial effects). For example, several divided doses may be administered daily or the dose may be proportionally reduced as indicated by the exigencies of the therapeutic situation.

The terms “transcription activation domain” and “TAD” are used interchangeably herein and refer to a regulatory element that stimulates transcription in cells. A transactivating domain is generally selected that is stable and provides enhanced expansion of stem cells. A transcription activation domain does not include a transcription activation domain of a homeobox polynucleotide, i.e. a HOX-TAD. Therefore, in the context of the present invention a TAD is a non-HOX-TAD. Examples of TADs include a transcription activation domain comprising a FG repeat region, a GAL4 transcription activating domain (Brent, R., Cell, 1985, 43: 729-736), viral VP16 activation domain, in particular the HSV VP16 activation domain, (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B [Bitko & Barik, J. Virol. 72:5610-5618 (1998) and Doyle & Hunt, Neuroreport 8:2937-2942 (1997)); Liu et al., Cancer Gene Ther. 5:3-28 (1998)], artificial chimeric functional domains such as VP64 (Seifpal et al., EMBO J. 11, 4961-4968 (1992)); and, a transcription factor module of Gal4-VP16 (Sadowski et al. (1988) Nature 335, 563-564). Additional exemplary activation domains include, but are not limited to, VP64, p300, CBP, PCAF, SRC1 PvALF, AtHD2A, ERF-2, OsGAI, HALF-1, Cl, AP1, ARF-5, -6, -7, and -8, CPRF1, CPRF4, MYC-RP/GP, and TRAB1 [See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504; Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15, 353.]

In aspects of the invention the transcription activation domain is a transcription activation domain comprising a FG repeat region, in particular the TAD is a nucleoporin.

“Test substance” includes but is not limited to proteins, peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], polynucleotides, ribozymes, carbohydrates, and small organic or inorganic molecules. A test substance may be an endogenous physiological compound or it may be a natural or synthetic compound.

A “translocating polypeptide” refers to a polypeptide or functional fragment thereof that transduces or crosses biological membranes, such as cell membranes. Translocating polypeptides, functional fragments thereof that have translocating properties, and polypeptide variants thereof, can possess one or more of the following properties: resistance to proteolysis, receptor-independent penetration of cell membranes, and substantially energy-free penetration of cell membranes. A translocating polypeptide can be used to deliver a chimeric polypeptide or a homeobox polypeptide and a TAD (e.g. nucleoporin) to the interior of a target cell (e.g. stem cell) either in vitro or in vivo.

Examples of translocating polypeptides include VP22 from Herpes Simplex Virus type 1 (see Elliot G. and P. O'Hare, Cell 88:223-233, 1997; Phelan A et al., Nature Biotechnology 16:440-443, 1998; Dilber M S et al., Gene Therapy 6:12-21, 1999; Aints A. et al., Gene Med. 1:275-9, 1999; Wybranietz W A et al., J. Gene Med. 1:265-274, 1999; Derer W. et al., J. Mol. Med. 77:609-6138, 1999; PCT Publication No. WO 97/05265; U.S. Pat. No. 6,017,735 describes homologues; and, U.S. Pat. No. 6,017,735); HIV-1 Tat polypeptide or functional fragment thereof [e.g. protein transduction domain (PTD) (amino acids 49-57) RKKRRQRRR (SEQ ID NO. 37)] (Park J. et al, 2000, Mol Cells 13: 202-208; Fawell et al, 1994, Proc. Natl. Acad, Sci. USA 91:664-668; Nagahara et al, 1998, Nat. Med. 4:1449-1452; Schwarze et al, 1999, Science 285:1569-1572; Vocero-Akbani, 1999, Nat. Med. 5:29-33); a fragment of the Antennapedia protein from Drosophila (Antp) (amino acids 43 through 58) (5′-RQIKIWFQNRRMKWKK-3′ SEQ ID NO. 38) (Derossi, D. et al., J. Biol. Chem. 269:10444-10450 1994; Axcrona et al, 1999), and Protein H from Streptococcus pyogenes (Derossi, D., et al, J. Biol. Chem. 271:18188-93), and the like. The general application of translocating polypeptides or fragments thereof is to deliver other molecules to cells, by incorporating the polypeptide or functional fragments thereof in a chimeric polypeptide or attaching a desired molecule(s) to the translocating protein. In a chimeric polypeptide, a translocating polypeptide can be located either in the N-terminal or C-terminal position. In aspects of the invention, small protein transduction domains (PTDs) from a translocating polypeptide can be fused to a homeodomain polypeptide, nucleoporin, or chimeric polypeptide of the invention to transport the polypeptides into a cell (e.g. stem cell). In particular aspects of the invention the translocating polypeptide is a HIV-1 TAT polypeptide or functional fragment thereof.

A translocating polypeptide can be covalently attached or attached by means of a linker to a desired molecule(s). Examples of linkers that may be fused to translocating polypeptides include peptide linkers [e.g. polylysine sequences and sequences containing three or more repeats of the peptide sequence LARL, in particular LARL-LARL-LARL (Fritz J D et al, Hum. Gene Ther. 7:1395-1404, 1996]. In some aspects of the invention a translocating polypeptide is fused to a protein domain that readily associates with a cationic liposome (e.g., a hydrophobic transmembrane domain or a glycosyl phosphatidylinositol anchor).

The term “treating” refers to reversing, alleviating, or inhibiting the progress of a condition and/or disease, or one or more symptoms of such condition and/or disease, to which such term applies. Depending on the condition of the subject, the term also refers to preventing a condition and/or disease, and includes preventing the onset, or preventing the symptoms associated with a condition and/or disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition of the present invention to a subject that is not at the time of administration afflicted with the disease. “Preventing” also refers to preventing the recurrence of a disease or of one or more symptoms associated with such disease. The terms “treatment” and “therapeutically,” refer to the act of treating, as “treating” is defined above.

### Constructs, Vectors, Cells, and Chimeric Polypeptides

The invention provides an isolated nucleic acid construct comprising a homeobox polynucleotide and a sequence encoding a TAD (e.g. nucleoporin polynucleotide). The N-terminal region of a sequence encoding a TAD (e.g. nucleoporin polynucleotide) can be fused to the C-terminal (homeodomain containing region) of a homeobox polynucleotide.A homeobox polynucleotide and sequence encoding a TAD (e.g. nucleoporin polynucleotide) can be selected that provide stem cells with enhanced capacity to undergo substantial self-renewal and the ability to give rise to all hematopoietic cell lineages.

In an aspect of the invention, the homeobox polynucleotide is a HOX gene, more particularly a HOX gene of the Antennapedia class or Abdominal B class. In a particular aspect, an isolated nucleic acid construct of the invention comprises a HOXB4, HOXA9, HOXA10, HOXD13, or HOXB3 polynucleotide, or fragment thereof (e.g. homoebox), more particularly HOXA10, HOXD13 or fragments thereof (e.g. DNA binding domain or homeodomain alone).

Nucleic acid constructs of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

A homeobox polynucleotide and sequence encoding a TAD (e.g. nucleoporin polynucleotide) may be inserted into a vector that contains the necessary regulatory elements for the transcription and translation of the inserted sequences. Accordingly, vectors adapted for transformation of a host cell (e.g. stem cell) may be constructed which comprise a homeobox polynucleotide and sequence encoding a TAD (e.g. nucleoporin polynucleotide), and one or more regulatory elements necessary for transcription and translation, operably linked to a nucleic acid sequence in the construct. Vectors can be prepared using techniques well known to those skilled in the art (see for example, Sambrook et al.). Possible expression vectors include but are not limited to cosmids, plasmids, phages, or modified viruses (e.g. replication defective retroviruses, adenoviruses and adeno-associated viruses), so long as the vector is compatible with the host cell used. Selection of appropriate regulatory elements is dependent on the host cell chosen and may be readily accomplished by one of ordinary skill in the art. The necessary regulatory elements may be supplied by the native sequence and/or its flanking regions.

A nucleic acid construct can also comprise additional sequences such as sequences that enhance or facilitate delivery of a polypeptide or polynucleotide, exogenous genes, and/or sequences encoding proliferation factors. In aspects of the invention a nucleic acid construct comprises a sequence encoding a translocating polypeptide or functional fragment thereof, in particular a HIV-1 Tat, more particularly the nine amino acids of the Tat PTD domain.

A nucleic acid construct or vector may also contain a gene encoding a detectable substance which facilitates the selection of host cells transformed or transfected with a nucleic acid construct of the invention. The markers can be introduced on a separate vector from the nucleic acid construct.

In addition a nucleic acid construct or vector may contain a sequence encoding an epitope tag to facilitate detection, isolation, and/or purification of a polypeptide produced using the construct. Examples of epitope tags include, without limitation, Flag-tag, His-tag, and GST-tag.

In an aspect of the invention, a nucleic acid construct comprises a sequence encoding a nucleoporin fused to a FLAG sequence which is fused to a homeobox polynucleotide or fragment thereof. In an embodiment, the nucleoporin is NUP98 and the homeobox polynucleotide is HOXA10, HOXD13, HOXA9, or HOXB3 or a fragment thereof (e.g. homeobox), in particular HOXA10 or a fragment thereof.

In a particular embodiment, a NUP98-homoebox polynucleotide construct is provided comprising a FLAG sequence fused to NUP98 through a BamHI site of SuperCatch-NUP98. A NUP98 sequence can be fused to a homeobox polynucleotide through an engineered site (e.g. EcoRI site) at the breakpoint site in NUP98.

In another particular embodiment, a NUP98-HOXB4 construct comprises the second exon of HOXB4 and an engineered site (e.g. EcoRI site) added upstream of the start of the HOXB4 second exon. More particularly, a FLAG sequence can be fused to a NUP98 sequence and the whole second exon of HOXB4 fused to NUP98 at an engineered site (e.g. EcoRI site). In a further particular embodiment, a NUP98-HOXB4 construct is provided which includes a PIM of HOX B4 starting upstream (e.g. about 14 codons) of the PIM.

Examples of nucleic acid constructs that can be utilized in the present invention include NUP98-HOXD13 (with or without a FLAG sequence), NUP98-HOXD13 (homeodomain of HOXD13 only), NUP98-HOXB4, NUP98-HOXB4-PIM (with or without a FLAG sequence), NUP98-HOXA10, NUP98-HOXA10-PIM (with or without a FLAG sequence), NUP98-HOXA10 (homeodomain of HOXA10 only), NUP98-HOXB3 (with or without a FLAG sequence), and NUP98-HOXB3 (homeodomain of HOXB3 alone). Sequences for particular nucleic acid constructs of the invention are in SEQ ID NOs. 16, 18, 20, 22, 24, and 25.

A nucleic acid construct or vector of the invention can be used to prepare transformed host cells comprising a nucleic acid construct or expressing a chimeric polypeptide comprising a homeodomain polypeptide and a nucleoporin. Nucleic acid constructs or vectors can also be used to transform host cells with an exogenous homeobox polynucleotide and an exogenous sequence encoding a TAD (e.g. nucleoporin polynucleotide) Therefore, the invention further provides host cells comprising or transformed with a nucleic acid construct or vector(s) described herein. Constructs or vectors can be introduced into a cell by one of many standard techniques known in the art.

Suitable host cells include a wide variety of prokaryotic and eukaryotic host cells. In reference to embodiments described herein the host cell is a stem cell. Therefore, in accordance with an aspect of the invention a stem cell can be modified to express a nucleic acid construct of the invention or a homeobox polynucleotide and a sequence encoding a TAD (e.g. nucleoporin polynucleotide). Thus, the invention contemplates a stem cell modified to express a nucleic acid construct of the invention or a homeobox polynucleotide and a sequence encoding a TAD (e.g. nucleoporin polynucleotide). Also contemplated are cell preparations comprising stem cells modified to express a nucleic acid construct of the invention or an exogenous homeobox polynucleotide and a sequence encoding a TAD (e.g. nucleoporin polynucleotide). A stem cell of the invention may be modified by any means known in the art which results in stable integration and expression of a nucleic acid construct or polynucleotides in the modified cell and its progeny (e.g., see for example method disclosed herein).

The invention also contemplates a cell line comprising modified stem cells expressing a nucleic acid construct of the invention or an exogenous homeobox polynucleotide and a sequence encoding a TAD (e.g. nucleoporin polynucleotide), and transgenic non-human mammals whose germ cells and somatic cells comprise a nucleic acid construct of the invention, or an exogenous homeobox polynucleotide and a sequence encoding a TAD (e.g. nucleoporin polynucleotide) of the invention.

The invention also permits the construction of nucleotide probes that are unique to a nucleic acid construct of the invention. A probe may be labeled, for example, with a detectable substance and it may be used to select nucleic acid constructs of the invention. A probe may be used to mark cells comprising a nucleic acid construct or chimeric polypeptide of the invention.

The invention further provides a method for preparing a chimeric polypeptide encoded by a nucleic acid construct of the invention or comprising a homeodomain polypeptide and a TAD (e.g. nucleoporin) utilizing a purified and isolated nucleic acid construct, vectors or host cells of the invention. In an embodiment a method for preparing a chimeric polypeptide comprising a homeodomain polypeptide and a TAD (e.g. nucleoporin) is provided comprising (a) transferring a vector of the invention into a host cell; (b) selecting transformed host cells from untransformed host cells; (c) culturing a selected transformed host cell under conditions which allow expression of a chimeric polypeptide; and (d) isolating the chimeric polypeptide. The invention also relates to chimeric polypeptides prepared by a process of the invention.

Chimeric polypeptides of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

The invention provides a chimeric polypeptide comprising a homeodomain polypeptide and a nucleoporin. In an aspect the invention provides a chimeric polypeptide comprising a part derived from a homeodomain polypeptide fused to a nucleoporin.

Examples of chimeric polypeptides of the invention include nucleoporin 98-homeodomain polypeptide HoxD13, nucleoporin 98-homeodomain polypeptide HoxB4, nucleoporin 98-homeodomain polypeptide HoxB4-PIM, nucleoporin 98-homeodomain polypeptide HoxA10 with or without a PIM, and nucleoporin 98-homeodomain polypeptide HoxB3 with or without a PIM. A homeodomain polypeptide of a chimeric polypeptide of the invention can be the homeodomain only. Sequences for particular isolated chimeric polypeptides of the invention are in SEQ ID NOs. 16, 18, 20, and 22.

In some aspects of the invention a chimeric polypeptide of the invention may comprise an epitope tag (e.g. Flag-tag, His-tag, or GST-tag) to facilitate detection, isolation, and/or purification of the chimeric polypeptide.

In other aspects of the invention a chimeric polypeptide of the invention may also comprise an element that enhances or facilitates delivery of a polypeptide. In particular, a chimeric polypeptide can comprise a translocating polypeptide or functional fragment thereof, more particularly a HIV-1 Tat, most particularly a Tat PTD domain.

The invention further contemplates antibodies having specificity against an epitope of a chimeric polypeptide of the invention. Antibodies can be prepared which bind a distinct epitope in an unconserved or conserved region of a polypeptide, preferably conserved region. Antibodies may be labeled with a detectable substance and used to detect chimeric polypeptides of the invention in cells.

### Compositions

The present invention relates to a composition comprising a homeodomain polypeptide and a nucleoporin, a chimeric polypeptide, an Abd-B like Hox polypeptide or polynucleotide, a nucleic acid construct, modified stem cells comprising a nucleic acid construct, or expanded cell preparations of the invention, and optionally a pharmaceutically acceptable carrier, excipient or diluent. A pharmaceutical composition may include a targeting agent to target cells to particular tissues or organs.

A composition of the invention can also a product of an exogenous gene (e.g., a therapeutic) and/or a proliferation factor.

A homeodomain polypeptide and a nucleoporin, a chimeric polypeptide, modified stem cells, an Abd-B like Hox polypeptide or polynucleotide, a nucleic acid construct, a homebox polynucleotide and a sequence encoding a TAD (e.g. nucleoporin polynucleotide), or expanded cell preparations of the invention may be formulated into pharmaceutical compositions for administration to subjects in a biologically compatible form suitable for administration. By “biologically compatible form suitable for administration” is meant a form in which any toxic effects are outweighed by the therapeutic effects. Dosage regimens may be adjusted to provide the optimum therapeutic response. For example, several divided doses may be administered daily or the dose may be proportionally reduced as indicated by the exigencies of the therapeutic situation.

The compositions described herein can be prepared by per se known methods for the preparation of pharmaceutically acceptable compositions which can be administered to subjects, such that a therapeutically effective amount of the cells/polypeptides is combined in a mixture with a pharmaceutically acceptable vehicle. Suitable vehicles are described, for example, in Remington's Pharmaceutical Sciences, 19^{th} Edition (Mack Publishing Company, Easton, Pa., USA 1995). On this basis, the compositions include, albeit not exclusively, solutions of the cells/polypeptides in association with one or more pharmaceutically acceptable vehicles or diluents, and contained in buffered solutions with a suitable pH and iso-osmotic with the physiological fluids.

Aspects of the invention provide compositions for in vitro or in vivo delivery of a homeodomain polypeptide and TAD (e.g. nucleoporin) or chimeric polypeptide of the invention. In particular, the invention contemplates a pharmaceutically acceptable delivery composition comprising a homeodomain polypeptide and a TAD (e.g. nucleoporin), or a chimeric polypeptide, and an element that enhances or facilitates delivery of the polypeptides to stem cells. The element can be an intracellular delivery vehicle operatively associated with a polypeptide to be delivered. Thus, the invention provides compositions for intracellular delivery of homeodomain polypeptides and TADs (e.g. nucleoporin), or chimeric polypeptides comprising an intracellular delivery vehicle associated with the polypeptides, wherein the vehicle upon contact with a cell membrane effects intracellular delivery of the associated polypeptide. A polypeptide can be directly linked to the vehicle or linked via a linker.

In accordance with one aspect a composition is provided comprising a homeodomain polypeptide and a TAD (e.g. nucleoporin) each in operative association with an intracellular delivery vehicle including a translocating polypeptide or cationic delivery vehicle (e.g. cationic lipid, cationic liposome, a lipoplex, or an anionic polymer).

The intracellular delivery vehicle can be a translocating polypeptide. In an embodiment, a composition of the invention provides a chimeric polypeptide comprising a translocating polypeptide for transport of the chimeric polypeptide across a cell membrane (e.g. TAT or VP22). In a particular embodiment the translocating polypeptide is HIV-1 Tat or a functional fragment thereof (e.g. Tat PTD).

An intracellular delivery vehicle can be a cationic delivery vehicle. In embodiments of the invention, compositions are provided for intracellular delivery of a homeodomain polypeptide and a nucleoporin, or a chimeric polypeptide comprising a cationic delivery vehicle in operative association with the polypeptide and nucleoporin, or chimeric polypeptide. A cationic delivery vehicle is adapted to fuse with a cell membrane to effect intracellular delivery of the associated protein. A delivery vehicle may be directly linked or indirectly linked through a linker to a polypeptide to be delivered.

An intracellular delivery vehicle can be a polynucleotide (RNA or DNA). In an aspect of the invention a composition of the invention comprises a homeodomain polypeptide and a nucleoporin, or a chimeric polypeptide, and a polynucleotide. In an embodiment, a composition is provided comprising (a) a polynucleotide, (b) a linker comprising a reactive group capable of binding to a polypeptide; (c) a homeodomain polypeptide and a TAD (e.g. nucleoporin), or chimeric polypeptide, each capable of binding to, or bound to a reactive group of the linker, and (c) a cationic lipid. In particular the linker is a peptide nucleic acid (PNA) bound to the polynucleotide, wherein the PNA includes a reactive group capable of binding to the polypeptide. Other heterobifunctional linkers include but are not limited to imidoesters, N-hydroxysuccinimide (NHS)-esters such as SMCC and succimidyl-4-(p-maleimidophenyl)-butyrate, maleimides, haloacetyls, pyridyl disulfides, carbodiimide crosslinkers (e.g. 1-ethyl-3-(3-dimethylaminopropyl-carbodihnide hydrochloride.

A pharmaceutically acceptable delivery composition can be used in the form of a solid, a solution, an emulsion, dispersion, a micelle, a liposome, and the like, in admixture with an organic or inorganic carrier or excipient suitable for administration to stem cells in vitro or in vivo.

The invention relates to a method of making a polypeptide delivery composition for enhancing expansion of stem cells, comprising combining each of a homeodomain polypeptide and a nucleoporin, or a chimeric polypeptide with an intracellular delivery vehicle including a translocating polypeptide or a cationic delivery vehicle.

The compositions can be indicated as proliferation or therapeutic agents either alone or in conjunction with other proliferation factors, therapeutics, or other forms of treatment (e.g. chemotherapy or radiotherapy). For example, the compositions may be used in combination with proliferation factors, anti-proliferative agents, antimicrobial agents, immunostimulatory agents, or anti-inflammatories. The compositions of the invention may be administered concurrently, separately, or sequentially with other factors, therapeutic agents, or therapies.

A composition of the invention or components utilized in methods of the invention may be supplied as a kit comprising a container that comprises one or more nucleic acid construct, a homeobox polynucleotide and a sequence encoding a TAD (e.g. nucleoporin polynucleotide), an Abd-like HOX polynucleotide or polypeptide, chimeric polypeptide, a homeodomain polypeptide and a nucleoporin, or a composition of the invention. A kit can further comprise a pharmaceutically acceptable carrier, excipient, or vehicle. In addition, a kit can comprise written information on indications and usage of the components.

In an aspect, the invention provides a kit for expanding stem cells, containing a composition comprising one or more nucleic acid construct, a homeobox polynucleotide and a sequence encoding a TAD (e.g. nucleoporin polynucleotide), a homeodomain polypeptide and a nucleoporin, a Abd-B-like HOX polynucleotide or polypeptide, chimeric polypeptide, or composition of the invention or components thereof, a container, and instructions for use.

Kits comprising therapeutic polypeptides can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide.

Cells or cell preparations of the invention can be packaged and distributed separately or in separate containers in kit form, or for simultaneous administration to the same site they can be mixed together. Sets of cells existing at any time during their manufacture, distribution, or use are also contemplated herein. Cell sets may comprise any cell populations described herein, alone or in combination with other cell types. Each cell type in a set may be packaged together, in separate containers at the same or different facilities, under the control of the same of different entities.

### Applications

The present invention in part is based on a finding that the expression of nucleic acid constructs, Abd-B-like HOX polynucleotides or polypeptides, chimeric polypeptides, compositions, or a combination of a TAD (e.g. nucleoporin) and a homeodomain polypeptide described herein has unique and unexpected effects on stem cells. Long-term repopulating stem cells engineered to express the nucleic acid constructs, a homeobox polynucleotide and a sequence encoding a TAD (e.g. nucleoporin polynucleotide), or a Abd-B-like HOX polynucleotide, or that contain a chimeric polypeptide, Abd-B-like Hox polypeptide, composition of the invention, or a combination of a TAD (e.g. nucleoporin) and a homeodomain polypeptide generate an expanded population of cells with the ability to undergo substantial self-renewal and the ability to give rise to different cell lineages, in particular hematopoietic cell lineages. The effect on cell expansion results in no discernable effect on differentiation following transplantation into recipients.

The invention provides methods to expand stem cells producing large numbers of cells that can be used for a number of research, development, and conunercial purposes. Of particular interest are uses of the constructs, compositions, and methods of the invention for clinical therapy of hematopoietic pathology, inducing immune tolerance, and for drug development.

The ability of a nucleic acid construct, a homeobox polynucleotide and a sequence encoding a TAD (e.g. nucleoporin polynucleotide), Abd-B-like HOX polynucleotide, Abd-B-like Hox polypeptide, chimeric polypeptide, a homeodomain polypeptide and a nucleoporin, or a composition or combination described herein to enhance the proliferative capacity of a primitive stem cell subpopulation without loss of pluripotent capacity has important clinical implications for enhancement or restoration of stem cell capability in subjects in which such capability is lost or threatened.

The invention features methods for enhancing expansion of stem cells or regenerative potential of stem cells in vivo. According to an aspect of the invention a method is provided for increasing regenerative potential of long-term repopulating stem cells in vivo comprising administering a nucleic acid construct, a homeobox polynucleotide and a sequence encoding a TAD (e.g. nucleoporin polynucleotide), Abd-B-like HOX polynucleotide, Abd-B-like Hox polypeptide, chimeric polypeptide, a homeodomain polypeptide and a nucleoporin, composition, or combination described herein to the stem cells.

The invention also provides a method for repopulating stem cells in a subject comprising delivering to stem cells in the subject a nucleic acid construct, a homeobox polynucleotide and a sequence encoding a TAD (e.g. nucleoporin polynucleotide), Abd-B-like HOX polynucleotide, Abd-B-like Hox polypeptide, chimeric polypeptide, a homeodomain polypeptide and a nucleoporin, composition, or combination described herein.

In an aspect the stem cells are transduced with a nucleic acid construct, a homeobox polynucleotide and a sequence encoding a TAD (e.g. nucleoporin polynucleotide) or an Abd-B-like HOX polynucleotide. In an embodiment, the invention provides a method for reconstituting or repopulating hematopoietic cells in a patient comprising administering to the patient a HOX polynucleotide selected from the group consisting of a class HOXA, HOXB, HOXC, and HOXD gene at a dose lower than hereto before administered, in particular at a dose substantially lower than contemplated in U.S. Pat. No. 5,837,507. An embodiment of a method for reconstituting or repopulating hematopoietic cells in a patient comprises administering a dose which is 20 to 200 fold lower than the conventional dose.

In particular aspects of the invention, nucleic acid constructs, a homeobox polynucleotide and a sequence encoding a TAD (e.g. nucleoporin polynucleotide), or Abd-B-like HOX polynucleotide may be introduced into stem cells using viral gene delivery systems for example, derived from retroviruses, adenoviruses, herpes or vaccinia viruses or from various bacterial plasmids for delivery of nucleic acid constructs to the target organ, tissue, or cells. In viral delivery methods, vectors may be administered to a subject by injection, e.g. intravascularly or intramuscularly, by inhalation, or other parenteral modes. Non-viral delivery methods include administration of the polynucleotides or nucleic acid constructs using complexes with liposomes or by injection; a catheter or biolistics may also be used. In an aspect, a retroviral gene delivery system is used which results in a high rate of gene transfer and stable integration of the genetic material which ensures the progeny of the modified cell will contain the transferred genetic material.

Administration to a subject of a composition of the invention, a homeodomain polypeptide and a nucleoporin, or a chimeric polypeptide, and optionally an element that enhances or facilitates delivery of polypeptides into stem cells and/or a targeting agent to target the polypeptides or compositions to stem cells (e.g., antibodies specific for markers on stem cells), can be intravenous, intra-arterial, intraperitoneal, intramuscular, subcutaneous, intrapleural, intrathecal, by perfusion through a regional catheter, by direct intralesional injection, oral, mucosal-membrane, pulmonary, and transcutaneous.

For oral delivery polyester microspheres, zein microspheres, proteinoid microspheres, polycyanoacrylate microspheres, and lipid-based systems may be suitable (see for example, DiBase and Morrel, “Oral Delivery of Microencapsulated Proteins”, in Protein Delivery: Physical Systems, Sanders and Hendren (eds.), pages 255-288 (Plenum Press 1997). Intranasal delivery may be suitable; for example, dry or liquid particles of a composition of the invention or polypeptides therein can be prepared and inhaled with the aid of dry-powder dispersers, liquid aerosol generators, or nebulizers (for example, see Pettit and Gombotz, TIBTECH 16:343, 1998; Patton et al, Adv, Drug Deliv. Rev. 32:235, 1999; and AERX diabetes management system which is a hand-held electronic inhaler that delivers insulin). Compositions of the invention or polypeptides therein can be delivered across skin at therapeutic concentrations with the aid of low-frequency ultrasound (see Mitragorti et al, Scinece 269:850, 1995), or transdermally using electroporation (Potts et al, Pharm. Biotechnol. 10:213, 1997).

A composition of the invention or components thereof, chimeric polypeptides, or a homeodomain polypeptide and a nucleoporin, can be encapsulated within liposomes using standard techniques of protein microencapsulation (see, for example, Anderson et al, Infect Immun 31:1099, 1981; Anderson et al., Cancer Res. 50:1853, 1990; and Cohen et al, Biochim Biophys. Acta. 1063:95, 1991; Alving et al, “Preparation and Use of Liposomes in Immunological Studies,” in Lipsome Technology, 2^{nd} Edition, Vol. III, Gregoriadis (ed.), page 317 (CRC Press 1993), Wassef et al, Meth. Enzymol. 149:124, 1987). Liposomes provide a means to deliver the compositions or components thereof to a subject intravenously, intraperitoneally, intrathecally, intramuscularly, subcutaneously, or by oral administration, inhalation, or intranasal administration. Liposomes can be prepared to target particular cells or organs by varying the phospholipid composition or binding targeting agents to the surface of the liposome such as antibodies, carbohydrates, vitamins, and transport proteins.

Degradable polymer microspheres may be used to maintain high systemic levels of a composition of the invention or components thereof. Microspheres can be prepared from degradable polymers including poly(lactide-co-glycolide)(PLG), polyanhydrides, poly(ortho esters), nonbiodegradable ethylvinyl acetate polymers, in which polypeptides are entrapped in the polymer (see for example, Gombotz and Pettit, Bioconjugate Chem. 6:332, 1995; Ranade, “Role of Polymers in Drug Delivery: in Drug Delivery Systems, Ranade and Hollinger (eds.), pages 51-93 (CRC Press, 1995); Roskos and Maskiewicz, “Degradable Controlled Release Systems Useful for Protein Delivery,” in Protein Delivery: Physical Systems, Sanders and Hendren (eds.), pages 45092 (Plenum Press 1997); Barus et al., Science 281: 1161, 1998; Putney an Burke, Nature Biotechnology 16:153, 1998; Putney, Curr. Opin. Chem. Biol. 2:548 (1998). Nanospheres coated with polyethylene glycol can also provide carriers for intravenous administration of a homeodomain polypeptide and a nucleoporin, or a composition of the invention or components thereof (see for example, Gref et al., Pharm Biotechnol. 10: 167, 1997).

Other dosage forms for delivering a composition of the invention and components thereof can be devised by a person skilled in the art (for example, see Remington's Remington's Pharmaceutical Sciences, 19^{th} Edition (Mack Publishing Company, Easton, Pa., USA 1995).

The invention features methods for enhancing expansion or regenerative potential of stem cells in a subject by modifying stem cells in culture. These methods involve administering to a subject, stem cells that have been expanded in vitro and/or transplanting transduced cells in a subject that have a competitive advantage. General procedures for clinical applications of hematopoietic cells are described in standard textbooks, including the Textbook of Internal Medicine, 3d Edition, by W. N. Kelly (ed.), (Lippincott-Raven, 1997), and in publications including Hematopoietic Stem Cell Transplantation, by A. D. Ho et al.(eds.)., Blackwell Science Inc, 1999; Hematopoietic Stem Cell Therapy, E. D. Ball, (J. Lister & P. Law, (Churchill Livingstone, 2000). Other clinical uses that will occur to a clinical practitioner are also within the scope of this invention.

In an aspect, the invention relates to a method for providing stem cells (e.g. primitive bone marrow cells) with increased regenerative potential in vivo by modifying the stem cells to express a nucleic acid construct of the invention, a homeobox polynucleotide and a sequence encoding a TAD (e.g. nucleoporin polynucleotide), a homeodomain polypeptide and a nucleoporin, a chimeric polypeptide, a composition of the invention, or a Abd-B-like HOX polynucleotide or polypeptide. This potential may be exploited through in vitro cultures to expand stem cells and/or following in vivo transplantation where transduced cells have a competitive proliferative advantage, resulting in significantly greater reconstitution of the stem cell compartment. This is particularly useful for re-establishing hematopoietic capability in subjects in which native hematopoietic function has been partially, substantially, or completely compromised.

According to another aspect of the invention there is provided a method of hematopoietic cell transplantation. The method is effected by (a) obtaining from a donor hematopoietic stem cells to be transplanted; (b) modifying the hematopoietic stem cells with a nucleic acid construct of the invention, a homeobox polynucleotide and a sequence encoding a TAD (e.g. nucleoporin polynucleotide), a homeodomain polypeptide and a nucleoporin, a chimeric polypeptide, a composition of the invention, or a Abd-B-like HOX polynucleotide or polypeptide; (c) culturing the modified stem cells under proliferation conditions to thereby expand the stem cells; and (d) transplanting the expanded stem cells in a patient. The transplanted cells can be administered in a pharmaceutical composition as described herein.

The invention provides a method of accelerating engraftment after transplantation in a subject comprising administering a modified stem cell of the invention to a subject in need thereof. In an aspect, the invention relates to a method of regenerating tissue in a subject comprising administering a modified stem cell of the invention to a subject in need thereof. In another aspect, the invention is directed to a method of recovering bone marrow in a patient suffering from loss of bone marrow cells comprising administering a modified stem cell of the invention to a subject in need thereof.

In a particular method of the invention, stem cells from any tissue are removed from a subject, modified by insertion of a nucleic acid construct, a homeobox polynucleotide and a sequence encoding a TAD (e.g. nucleoporin polynucleotide), or a Abd-B-like HOX polynucleotide, or introduction of a homeodomain polypeptide and a TAD (e.g. nucleoporin), chimeric polypeptide, or a composition, expanded in vitro by expression of the construct, polynucleotide(s), polypeptides, or composition, and returned to the subject. In the alternative, the modified stem cells can be returned to the subject without in vitro expansion. If necessary, the process may be repeated to provide substantial repopulation of the stem cells. The cells in the modified stem cell population returned to the subject retains pluripotent characteristics, e.g., self-renewal and ability to generate cells of all hematopoietic lineages. When in vitro expansion is desirable, a combination of various cytokines can be utilized to ensure that the transduced cell population in addition to the stem cells, includes expanded numbers of progenitor cells and more mature cells of the various hematopoietic lineages (e.g., megakaryocytes, neutrophils) to provide a cell population that will provide both short-term and long-term repopulation potential.

In another particular method of the invention, hematopoietic stem cells are removed from a human patient, and a population of stem cells isolated. These stem cells are modified by transduction with a vector comprising a construct of the invention, a homeobox polynucleotide and a sequence encoding a TAD (e.g. nucleoporin polynucleotide), or an Abd-B-like HOX polynucleotide, or by transduction with a homeodomain polypeptide and a TAD (e.g. nucleoporin), chimeric polypeptide, or a composition. The population of modified stem cells is then restored to the human patient with or without in vitro expansion. The patient may be treated to partially, substantially, or completely ablate the native hematopoietic capability prior to restoration of the modified stem cells. If necessary, the process may be repeated to ensure substantial repopulation of the modified stem cells.

Aspects of the invention provide methods for producing hematopoietic stem cells from embryonic stem cells, and methods for enhancing the output of hematopoietic stem cells from embryonic stem cells, in particular initiated or differentiated embryonic stem cells.

The invention contemplates a method of producing hematopoietic stem cells from embryonic stem cells comprising obtaining embryonic stem cells and modifying the stem cells with a homeodomain polypeptide, a homeodomain polypeptide and a TAD (e.g. a nucleoporin polypeptide), a nucleic acid construct, a homeobox polynucleotide, a homeobox polynucleotide and a sequence encoding a TAD (e.g. a nucleoporin polynucleotide), a composition of the invention or components thereof, chimeric polypeptide, or in particular an Abd-B-like HOX polynucleotide or polypeptide, so that the embryonic stem cells form hematopoietic stem cells. In an aspect of the invention, the embryonic stem cells are initiated embryonic stem cells. In other aspects of the invention, the embryonic stem cells are differentiated embryonic stem cells.

The invention also relates to methods for enhancing expansion or output of hematopoietic stem cells from embryonic stem cells. In particular a homeodomain polypeptide, a homeodomain polypeptide and a TAD (e.g. a nucleoporin polypeptide), a nucleic acid construct, a homeobox polynucleotide, a homeobox polynucleotide and a sequence encoding a TAD (e.g. a nucleoporin polynucleotide), a composition of the invention or components thereof, chimeric polypeptide, or in particular an Abd-B-like HOX polynucleotide or polypeptide are used to enhance output of hematopoietic stem cells from embryonic stem cells.

In an aspect, method for enhancing expansion of stem cells is provided comprising delivering to embryonic stem cells an effective amount of a homeodomain polypeptide, a homeodomain polypeptide and a TAD (e.g. a nucleoporin polypeptide), a nucleic acid construct, a homeobox polynucleotide, a homeobox polynucleotide and a sequence encoding a TAD (e.g. a nucleoporin polynucleotide), a composition of the invention or components thereof, chimeric polypeptide, or in particular an Abd-B-like HOX polynucleotide or polypeptide to provide enhanced expansion of stem cells from the embryonic stem cells.

Expression of a homeodomain polypeptide, a homeodomain polypeptide and a TAD (e.g. a nucleoporin polypeptide), a nucleic acid construct, a homeobox polynucleotide, a homeobox polynucleotide and a sequence encoding a TAD (e.g. a nucleoporin polynucleotide), a composition of the invention or components thereof, chimeric polypeptide, or in particular an Abd-B-like HOX polynucleotide or polypeptide, in embryonic stem cells results in enhanced ability of the stem cells to generate hematopoietic stem cells, and in particular expanded populations of hematopoietic stem cells.

The invention provides a method for expanding hematopoietic stem cells comprising obtaining embryonic stem cells; modifying the embryonic stem cells with a homeodomain polypeptide, a homeodomain polypeptide and a TAD (e.g. a nucleoporin polypeptide), nucleic acid construct, a homeobox polynucleotide, a homeobox polynucleotide and a sequence encoding a TAD (e.g. a nucleoporin polynucleotide), a composition of the invention or components thereof, chimeric polypeptide, or in particular an Abd-B-like HOX polynucleotide or polypeptide, and culturing and isolating increased numbers of hematopoietic stem cells.

Methods of the invention for enhancing expansion of stem cells may involve obtaining stem cells from a subject. Stem cells that can be expanded using the methods of the invention include stem and/or progenitor cells such as stem cells of hematopoietic cells, neural cells, oligodendrocyte cells, skin cells, hepatic cells, embryonic cells, muscle cells, bone cells, mesenchymal cells, pancreatic cells, chondrocytes, stromal cells, and stem cells derived from embryonic stem cells. In particular aspects, the stem cells can be obtained from bone marrow, peripheral blood or umbilical cord blood.

In aspects of the invention, stem cells may be obtained from peripheral blood of a subject. Methods for mobilizing stem cells into the peripheral blood are known in the art and can involve treatment with chemotherapeutic drugs, e.g., cytoxan, cyclophosphamide, VP-16, and cytokines such as GM-CSF, G-CSF, or IL-3, or combinations thereof. It will be appreciated that mobilizing stem cells into the peripheral blood may also be achieved using the constructs and chimeric polypeptides of the invention. Typically, removal of blood begins when the total white cell count reaches 500-2000 cells/μl and the platelet count reaches 50,000/μl. Leukapheris samples may be obtained daily and monitored for the presence of CD34⁺ cells to determine the peak of stem cell mobilization and, thus, the optimal time for harvesting peripheral blood stem cells.

Differentiated cells are preferably initially removed from a blood sample using a relatively crude separation, where the major populations of mature cells, such as lymphocytes, granulocytes, monocytes, megakaryocytic, mast cells, eosinophils, platelets, and basophils are removed. Generally, at least about 70 to 90 percent of differentiated cells are removed.

A subset of cells expressing the CD34 antigen (CD34⁺) can be obtained using negative and positive selection methods known in the art (Berenson et al. (1991) Blood 77:1717-1722). A fraction of CD34⁺ cells can be further subdivided based on additional antigen characteristics (Lansdorp et al. (1990) J. Exp. Med. 172:363-366; Verfaille et al. (1990) J. Exp. Med. 172:509-520; Briddell et al. (1992) Blood 79:3159-3167) including the lack of lineage specific markers (Lin⁻) (Baum et al. (1992) Proc. Natl. Acad. Sci. USA 89:2804-2808; Craig et al. (1993) J. Exp. Med. 177:1331-1342; Murray et al. (1990) Blood Cells 20:364-370; Murray et al. (1995) Blood 85:468). A separation technique used to obtain a specific fraction should maximize the viability of the fraction to be collected.

Separation techniques that can be used to obtain specific fractions of stem cells can be based on differences in physical (density gradient centrifugation and counter-flow centrifugal elutriation), cell surface (lectin and antibody affinity), and vital staining properties. Procedures for separation may include but are not limited to magnetic separation, using antibody-coated magnetic beads, affinity chromatography, cytotoxic agents joined to a monoclonal antibody or used in conjunction with a monoclonal antibody, including complement and cytotoxins, and “panning” with antibody attached to a solid matrix or any other convenient technique. Flow cytometry which can have varying degrees of sophistication, e.g., a plurality of color channels, low angle and obtuse light scattering detecting channels, impedance channels, etc. can also be used to provide specific stem cell fractions.

Embryonic stem cells used in methods of the invention can be isolated from blastocysts of members of the primate species (see, for example, Thomson et al., Proc. Natl. Acad. Sci. USA 92:7844, 1995; Thomson et al, Science 282:1145, 1998; Thomson et al., Curr. Top. Dev. Biol. 38:133ff., 1998; Ruebinoff et al., Nature Bitech. 18:399, 2000). Embryonic stem cells can also be isolated from preimplantation embryos, or in vitro fertilized embryos or one-cell embryos can be expanded to the blastocyst stage (see, for example, Bongso et al., Hum Reprod 4:706, 1989). Embryonic germ cells can be prepared from primordial germ cells present in fetal material.

Certain aspects of the invention utilize initiated embryonic stem cells. Initiated embryonic stem cells can be produced by culturing the cells under conditions to initiate differentiation in a non-specific way. For example, the cells can be cultured in differentiation medium causing the stem cells to form embryoid bodies or aggregates by overgrowth of an embryonic stem cell culture or by culturing the cells in suspension with a substrate with low adhesion properties. The undifferentiated stem cells are removed from culture, dissociated into clusters and cultured in a medium that supports differentiation or the undifferentiated stem cells are removed in strips and cultured in differentiation medium and aggregate into rounded cell masses. Factors that inhibit differentiation can also be withdrawn to initiate differentiation. Examples of other methods of non-specifically differentiating embryonic stem cells are known and include adding retinoic acid or dimethyl sulfoxide in the culture medium; not culturing cells on an extracellular matrix (see for example, WO 01/51616), or forming primitive ectoderm like cells (Rathjen et al., J. Cell Sci. 112:601, 1999).

Differentiated embryonic stem cells may also be used in certain methods of the invention. These cells can be produced by culturing undifferentiated or initiated embryonic stem cells in the presence of one or more hematopoietic differentiation factors. Examples of hematopoietic differentiation factors include hematogenic cytokines such as stem cell factor (SCF), interleukin 3 (IL-3), interleukin 6 (IL-6), granulocyte-colony-stimulating factor (G-CSF), either alone, or in combination with bone morphogenic proteins such as BMP-2, BMP-4, or BMP-7. Typically, at least two, three, or more than three hematapoietic factors are combined to create a differentiation cocktail. The undifferentiated or initiated embryonic stem cells are cultured in the factors for a sufficient time to permit the desired phenotype to emerge. It may also be beneficial to perform the culture over a substrate such as fibronectin, or the cells can be cocultured in the presence of stromal cells.

In methods of the invention, nucleic acid constructs or an Abd-B-like HOX nucleic acid can be introduced in stem cells (e.g. harvested stem cells) via conventional techniques as described herein. Suitable methods for transforming and transfecting cells can be found in Sambrook et al., and other laboratory textbooks. By way of example, a nucleic acid construct or a Abd-B-like HOX polynucleotide may be introduced into cells using an appropriate expression vector including but not limited to cosmids, plasmids, or modified viruses (e.g. replication defective retroviruses, adenoviruses and adeno-associated viruses). Transfection is easily and efficiently obtained using standard methods including culturing the cells on a monolayer of virus-producing cells.

Non-viral methods can also be used to introduce nucleic acid constructs or an Abd-B-like HOX polynucleotide in stem cells. Most non-viral methods of gene transfer rely on normal mechanisms used by mammalian cells for the uptake and transport of macromolecules. Non-viral methods include but are not limited to calcium phosphate or calcium chloride co-precipitation, DEAE-dextran-mediated transfection, lipofection, electroporation, or microinjection, liposomal derived systems, poly-lysine conjugates, and artificial viral envelopes.

Transduction of stem cells in vitro may be accomplished by the direct co-culture of stem cells with producer cells, following methods known in the art. For clinical applications, transduction by culturing the stem cells with viral supernatant alone or with purified viral preparations, in the absence of stromal cells, is preferred. Polycations, such as protamine sulfate, polybrene and the like, will generally be included to promote binding. Protamine sulfate and polybrene are typically used in the range of 4 μg/ml. Additionally, cytokines may also be added, including, e.g., IL-3, IL-6, LIF, steel factor (Stl) GM-CSF, G-CSF, MIP-1α., and Flk2/Flt3, preferably including Stl. The factors employed may be naturally occurring or synthetic, e.g., prepared recombinantly, and preferably human.

Expression of a nucleic acid construct, a homeobox polynucleotide or a sequence encoding a TAD (e.g. nucleoporin polynucleotide), or an Abd-B-like HOX polynucleotide in a modified stem cell can be controlled in a variety of ways. Thus, the nucleic acid construct, a homeobox polynucleotide or a sequence encoding a TAD (e.g. nucleoporin polynucleotide), or Abd-B-like HOX polynucleotide may be put under the control of a promoter that will cause the construct or polynucleotides to be expressed constitutively, only under specific physiologic conditions, or in particular cell types. Examples of promoters that may be used to cause expression of the introduced sequence in specific cell types include the CD34 promoter for expression in stem and progenitor cells, Granzyme A and Granzyme B for expression in T-cells and NK cells, the CD8 promoter for expression in cytotoxic cells, and the CDIIb promoter for expression in myeloid cells. Inducible regulatory elements may be used for gene expression under certain physiologic conditions. By appropriate use of an inducible regulatory element, expression of polypeptide products can be achieved in response to particular stimuli such as chemicals, chemo-attractants, particular ligands, and the like.

A gene encoding a detectable substance may be integrated into the stem cells for the identification of transformed cells. For example, a gene which encodes a protein such as β-galactosidase, chloramphenicol acetyltransferase, firefly luciferase, or a fluorescent protein marker may be integrated into the cells. Examples of fluorescent protein markers are the Green Fluorescent Protein (GFP) from the jellyfish A. Victoria, or a variant thereof that retains its fluorescent properties when expressed in vertebrate cells. (For example, the GFP variants described in references 22-24; and EGFP commercially available from Clontech Palo Alto, Calif.).

A sequence encoding an epitope tag may be integrated into the stem cells to facilitate detection, isolation, and/or purification of a polypeptide produced using the construct. Examples of epitope tags include, without limitation, Flag-tag, His-tag, and GST-tag.

Stem cells may be modified using a homeodomain polypeptide and nucleoporin, or a chimeric polypeptide, in particular a delivery composition thereof, using conventional methods. In an aspect, the invention provides a method of delivering a homeodomain polypeptide and TAD (e.g. nucleoporin) or chimeric polypeptide to a cell comprising contacting a cell membrane of a stem cell with a homeodomain polypeptide and a TAD (e.g. nucleoporin) each in association with a translocating polypeptide, or a chimeric polypeptide in association with a translocating polypeptide, such that the translocating polypeptide transports the polypeptide and nucleoporin, or chimeric polypeptide across the cell membrane.

Cationic delivery vehicles such as cationic lipids, negatively charged polymers, and cationic liposomes can also be used to deliver polypeptides into stem cells. Examples of such methods are described herein and in PCT Published Application No. WO 03095641 (Application WO 2003US0013873). Therefore, aspects of the invention include a method for delivering a homeodomain polypeptide and TAD (e.g. nucleoporin) or chimeric polypeptide to a cell comprising contacting a cell membrane of a stem cell with a homeodomain polypeptide and a TAD (e.g. nucleoporin) each in association with a cationic delivery vehicle, or a chimeric polypeptide in association with a cationic delivery vehicle, such that the delivery vehicle associates with the cell membrane and thereby delivers the polypeptide and nucleoporin, or chimeric polypeptide across the cell membrane. In particular, the vehicle is a cationic lipid which fuses with cell membrane to thereby allow the associated polypeptide to enter the cell.

In an embodiment, a homeodomain polypeptide and a TAD (e.g. nucleoporin), or a chimeric polypeptide can be introduced into stem cells by encapsulating the polypeptide(s) in a liposome. For example, an encapsulated polypeptide can be prepared by mixing a cationic lipid film and a polypeptide to be introduced into stem cells. Some of the polypeptide may become complexed with lipid and incorporated into the lipid phase. Examples of cationic lipids capable of encapsulating polypeptides include those described in U.S. Pat. Nos. 4,897,355; 5,264,618, and 5,459,127 and XG40 (see U.S. application Ser. No. 09/448,876). A co-lipid such as dioleoylphosphatidyl ethanolamine (DOPE), polyethyleneglycolphosphatidylethanolamine (PEG-PE), diphytanoyl-PE, cholesterol and monooleoylglycerol, may also be included in the reaction mixture. A mixture of the cationic lipid film and polypeptides can be added to cultured stem cells or introduced in vivo, and the polypeptide/lipid complexes associate with negatively charged cell surfaces. Following cell surface attachment, the liposomes fuse with the cell membrane and deliver encapsulated polypeptides into the cell. Liposomes can also be endocytosed and fused with the endosome releasing the encapsulated polypeptides into the cytoplasm.

Another polypeptide delivery method involves introducing a homeodomain polypeptide and a nucleoporin, or a chimeric polypeptide into stem cells comprising contacting the stem cells with one or more composition comprising a homeodomain polypeptide and a TAD (e.g. nucleoporin) or a chimeric polypeptide, a negatively charged polymer having a reactive group capable of coupling to the polypeptides, and a cationic liposome which interacts with the negatively charged polymer. Examples of polymers include polynucleotides (DNA or RNA, oligonucleotides), heparin, dextran sulfate, and polyglutamic acid. In an aspect the polymer is an oligonucleotide conjugated to available amino groups on a polypeptide to be delivered using for example a NHS-activated oligonucleotide. A polypeptide oligonucleotide complex can be transfected into stem cells using conventional lipid transfection reagents. A polypeptide can be conjugated to an oligonucleotide using heterobifunctional crosslinkers such as imidoesters, N-hydroxysuccinimide (NHS)-esters such as SMCC and succimidyl-4-(p-maleimidophenyl)-butyrate, maleimides, haloacetyls, pyridyl disulfides, carbodiimide crosslinkers (e.g. 1-ethyl-3-(3-dimethylaminopropyl-carbodihnide hydrochloride.

A homeodomain polypeptide and a TAD (e.g. nucleoporin) or a chimeric polypeptide can be introduced into stem cells by attaching the polypeptides to a polynucleotide (RNA or DNA), in particular a plasmid, and transfecting the plasmid into the cells with a conventional DNA transfection reagent. These types of complexes have been referred to as lipoplexes. In an aspect of the invention, a method is provided comprising delivering a homeodomain polypeptide and a TAD (e.g. a nucleoporin) or a chimeric polypeptide to stem cells comprising contacting the stem cells with one or more composition comprising (a) a polynucleotide, (b) a peptide nucleic acid (PNA) bound to the polynucleotide, wherein the PNA includes a reactive group capable of binding to the polypeptide, (c) a homeodomain polypeptide and a TAD (e.g. a nucleoporin) each bound to a reactive group of a PNA or a chimeric polypeptide bound to a reactive group of a PNA, and (c) a cationic lipid. The use of PNA clamps to attach polypeptides onto DNA is described in Zelphati et al., Biotechniques 28: 304-310, 2000; and PCT Publication No. WO9819503. The PNA clamp hybridizes with a complementary binding site on a plasmid to form a highly stable PNA-DNA-PNA triplex. Plasmids which can be utilized in this method include pGeneGrip (Gene Therapy Systems, Inc, San Diego, Calif.). Labeled PNA clamps may be used such as PNA labeled with reactive groups including biotin, maleimide and fluorescent labels (e.g., rhodamine and fluorescein). Methods using PNA clamps are described for example in PCT Published Application No. WO 03095641 (Application WO 2003US0013873).

A reverse protein delivery method can also be used for introducing a homeodomain polypeptide and a TAD (e.g. a nucleoporin) or a chimeric polypeptide into stem cells. In an aspect, a method is provided for transfecting stem cells with a homeodomain polypeptide and nucleoporin, or chimeric polypeptide using surface-mediated delivery. In an embodiment, a substrate surface having a polypeptide to be introduced into stem cells is used for culturing the stem cells in vitro. A polypeptide to be introduced into stem cells is pre-complexed with a carrier reagent before being applied to the surface. Stem cells are overlaid onto the prepared surface and the carrier reagent promotes the delivery of the polypeptide into the cells. In another embodiment, polypeptides to be introduced into stem cells are attached on a suitable substrate surface, a carrier reagent is added to the polypeptides to form complexes on the surface. In an embodiment, a polypeptide fused covalently to a translocating polypeptide is employed (e.g. a herpes simplex protein, VP22). In particular embodiments, a helper reagent is included to enhance the protein delivery efficiency.

Other methods for delivering polypeptides into cells can be utilized including electroporation, microinjection, methods using viral fusion proteins or cationic lipids, and methods devised by a person skilled in the art (see for example, Protein Delivery: Physical Systems, Sanders and Hendren (eds) (Plenum Press, 1997).

To ensure that the stem cells have been successfully modified, PCR may be used to amplify vector specific sequences in the transduced stem cells or their progeny. In addition, the cells may be grown under various conditions to ensure that they are capable of maturation to all of the hematopoietic lineages while maintaining the capability, as appropriate, of the introduced DNA. Various in vitro and in vivo tests may be employed to ensure that the pluripotent capability of the stem cells has been maintained. The stem cells can also be characterized based on tissue-specific markers using suitable immunological techniques or immunohistochemistry, microscopic observation of morphological features, functional criteria measurable in vitro, and behaviour upon infusion into a host animal.

Modified stem cells can be cultured using standard proliferation conditions. The stem cells may be cultured either with or without stromal cells. Stromal cells may be freshly isolated from bone marrow or from cloned stromal cell lines. Such lines may be human, murine, or porcine. For clinical applications, it is preferred to culture the stem cells in the absence of stromal cells. Expansion may be conducted with a variety of cytokines and growth factors, e.g., FLT-3 or steel factor. Various in vitro and in vivo tests known to the art may be employed to ensure that the pluripotent capability of the stem cells has been maintained.

The nucleic acid constructs, a homeobox polynucleotide and a sequence encoding a TAD (e.g. nucleoporin polynucleotide), Abd-B-like HOX polynucleotides, Abd-B-like Hox polypeptides, chimeric polypeptides, a homeodomain polypeptide and a nucleoporin, compositions and combinations described herein may be used to promote the survival of stem cells in in vitro culture. Thus, the invention contemplates a method for promoting survival of stem cells in culture comprising culturing the cells with a nucleic acid construct, a homeobox polynucleotide and a sequence encoding a TAD (e.g. nucleoporin polynucleotide), Abd-B-like HOX polynucleotide, Abd-B-like Hox polypeptide, chimeric polypeptide, a homeodomain polypeptide and a nucleoporin, a composition and combinations described herein.

The methods of the invention may be used to enhance in vivo regeneration by stem cells. Hematopoietic stem cells for autologous transplantation may be removed from a subject by aspiration or by mobilization for gene transfer of a homeobox polynucleotide or sequence encoding a TAD (e.g. nucleoporin polynucleotide) using direct transfection or various viral vectors. After the subject is treated (e.g., with chemotherapy or radiation therapy) the modified stem cells are re-injected into the subject and modified stem cells show accelerated engraftment and repopulation of bone marrow shortening the time of the aplastic window thereby decreasing the transplantation related mortality. Alternatively, the removed stem cells are incubated in a culture medium that contains a homeobox polypeptide and a TAD (e.g. a nucleoporin) comprising translocating polypeptides and the cultures cells are re-injected into the subject. The polypeptides delivered into the cells increase the ability of the cells to self renew and reconstitute in bone marrow.

Expanded stem cell preparations of the invention comprising increased numbers of stem cells may be used for enhancing the immune system of a subject. The cell preparations will facilitate enhancement or reconstitution of a subject's immune and/or blood forming system. In an aspect of the invention, the stem cell preparations of the invention are used in the treatment of leukemia (e.g. acute myelogenous leukemia, chronic myelogenous leukemia), lymphomas (e.g. non-Hodgkin's lymphoma), neuroblastoma, testicular cancer, multiple myeloma, melanomas, breast cancer, solid tumors that have a stem cell etiology, or other cancers in which therapy results in the depletion of hematopoietic cells.

In another aspect of the invention, a stem cell preparation of the invention, with or without genetic modification (see below) to provide resistance to HIV, is used to treat subjects infected with HIV-1 that have undergone severe depletion of their hematopoietic cell compartment resulting in a state of immune deficiency.

Modified stem cells or stem cells in an expanded cell preparation may be used in gene therapy. According to an aspect of the invention, modified stem cells or stem cells in an expanded cell preparation may be transfected with a desired exogenous gene, in particular a gene encoding a therapeutic, which can be used for treatment of neoplastic, infectious, or genetic diseases.

To perform gene therapy according to an aspect of the invention, modified cells or cell preparations comprising a therapeutic or drug are administered to a subject in need of the gene therapy, and then monitored biochemically and clinically for correction of the deficiency or dysfunction. Where possible it is preferable that the modified cells or cell preparations match the histocompatibility type of the cells being administered with the histocompatibility type of the subject. In particular, cells matched at the HLA-A, HLA-13, and HLA-DR loci are optimal. Where an exact histocompatibility match is not available, a match at one or two Class I or Class II loci is useful.

In an aspect, the stem cells may be modified to produce a product to correct a genetic deficiency, or where the host has acquired a genetic deficiency through a subsequent disease. For example, hematopoietic cell-related genetic diseases can be treated by grafting the expanded cell preparation with cells transfected with a gene that can make up for the deficiency or the abnormality of the gene causing the diseases. For example, a normal wild type gene that causes a disease such as β-thalassemia (Mediterranean anemia), sickle cell anemia, ADA deficiency, recombinase deficiency, recombinase regulatory gene deficiency and the like, can be transferred into the stem cells or by homologous or random recombination and the cells can be grafted into a patient. Further, an expanded preparation comprising normal hematopoietic stem cells free from abnormalities of genes (from a suitable donor) can be used for treatment.

Another application of gene therapy permits the use of a drug in a high concentration, which is normally considered to be toxic, by providing drug resistance to normal stem cells by transferring a drug resistance gene into the stem cells. In particular, it is possible to carry out the treatment using an anticancer drug in high concentration by transferring a gene having drug resistance against the anticancer drug, e.g., a multiple drug resistance gene into an expanded cell preparation comprising stem cells. For example, one may introduce genes that confer resistance to chemotherapeutic agents, thereby protecting the hematopoietic cells, allowing higher doses of chemotherapy and thereby improving the therapeutic benefit of treatment.

Diseases other than those relating to the hematopoietic system can be treated by using the expanded cell preparations of the invention in so far as the diseases relate to a deficiency of secretory proteins such as hormones, enzymes, cytokines, growth factors and the like. A deficient protein can be induced and expressed by transferring a gene encoding a target protein into a modified stem cell under the control of a suitable promoter. The expression of the protein can be controlled to obtain the same activity as that obtained by the natural expression in vivo.

For viral infections that primarily affect hematolymphoid cells, stem cells may be modified to endow the progeny with resistance to the infectious agent. In the case of human immunodeficiency virus (HIV), for example, specific antisense or ribozyme sequences may be introduced that interfere with viral infection or replication in the target cells. Alternatively, the introduced gene products may serve as “decoys” by binding essential viral proteins, thereby interfering with the normal viral life cycle and inhibiting replication. For example, stem cells can be subjected to gene modification to express an antisense nucleic acid or a ribozyme, which can prevent growth of hematic pathogens such as HIV, HTLV-I, HTLV-II and the like in the stem cells or cells differentiated from the stem cells.

The modified stem cells or expanded cell preparations comprising stem cells can be introduced in a vertebrate, which is a recipient of cell grafting, by conventional methods, for example, parenteral administration (e.g. intravenous and intra-arterial as well as other appropriate parenteral routes), subcutaneous administration, or transdermal administration. Cells or expanded cell preparations may be prepared for administration using standard techniques. The cells or expanded cell preparations may be supplied in the form of a pharmaceutical composition, comprising an isotonic excipient prepared under sufficiently sterile conditions for human administration. In particular, the cells or expanded cells may be prepared as a concentrated cell suspension in a sterile isotonic buffer. General procedures for formulating cell compositions are described, for example, in Cell Therapy: Stem Cell Transplantation, Gene Therapy, and Cellular Immunotherapy, by G. Morstyn & W. Sheridan eds., Cambridge University Press, 1996. Compositions and combinations of the invention intended for distribution are optionally packaged with written instructions for a desired purpose, such as the reconstitution of hematopoietic function or gene therapy.

The methods of the invention can be tested in well-established animal models. For example, repopulation of hematopoietic cells produced by a method of the invention for clinical application can be assessed in mice genetically engineered to forestall xenograft rejection. In particular, the NOD/SCID mouse containing the non-obese diabetic (NOD) genotype, crossed into mice with severe combined immunodeficiency (SCID) can be used to assess reconstitution. (See Larochelle et al., Nat. Med. 2:1329, 1996; Dick et al., Stem Cells 15:199, 1997; and Vormoor et al., J. Hematother. 2:215, 1993.) Hematopoietic cells produced by methods of the present invention can also be tested in less severely compromised immune systems, such as in non-irradiated NOD/SCID mice, SCID mice, nude mice, and immune competent mice.

According to an aspect of the invention a method is provided for conducting a regenerative medicine business, comprising: (a) a service for accepting and logging in samples from a client comprising stem cells; (b) a system for modifying and expanding cells dissociated from the samples in accordance with methods described herein; (c) a cell preservation system for preserving cells generated by the system in (b) for later retrieval on behalf of the client or a third party. The method may further comprise a billing system for billing the client or a medical insurance provider thereof.

The invention further relates to the use of modified stem cells and preparations comprising same, including expanded cell preparations, in drug discovery. Modified stem cells described herein can be used to screen for test substances (e.g., solvents, small molecule drugs, peptides, polynucleotides, or pharmaceutical compounds), or environmental conditions (e.g., culture conditions or manipulations) that affect proliferation of stem cells, or the characteristics of stem cells and their progeny. Thus, the invention provides a method for screening a test substance for its potential to affect proliferation or expansion of stem cells comprising:
(a) culturing modified stem cells comprising a nucleic acid construct, a homeobox polynucleotide and a sequence encoding a TAD (e.g. nucleoporin polynucleotide), Abd-B-like HOX polynucleotide, Abd-B-like Hox polypeptide, chimeric polypeptide, a homeodomain polypeptide and a nucleoporin, composition or combination described herein in the presence of the test substance or environmental condition;
(b) detecting the presence or absence of an effect of the test substance or environmental condition on expansion, or morphology, marker phenotype, or functional activity of the modified cells whereby an alteration in the amount of expansion indicates the test substance effects proliferation of stem cells.

Still another aspect of the present invention provides a method of conducting a drug discovery business comprising:
(a) providing one or more systems for identifying agents by their ability to inhibit or potentiate expansion of modified stem cells of the invention;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Modified stem cells and expanded cell preparations of the invention can be used in various bioassays. Different biological compounds (e.g. hormones, specific growth factors, etc.) can be added in a stepwise fashion to modified stem cells or expanded stem cell preparations to identify biological compounds that induce or inhibit proliferation or differentiation of stem cells. Other uses in a bioassay for the cells are differential display (i.e. mRNA differential display) and protein-protein interactions using proteins from the cells. Protein-protein interactions can be determined with techniques such as a yeast two-hybrid system. Proteins from modified cells and expanded cell preparations can be used to identify unknown proteins that interact with the cells including but not limited to growth factors, hormones, enzymes, transcription factors, translational factors, and tumor suppressors. Bioassays involving modified stem cells and expanded cell preparations of the invention, and the protein-protein interactions these cells form and the effects of protein-protein or cell-cell contact may be used to determine how surrounding tissue contribute to proliferation of hematopoietic cells.

In an aspect, the invention provides a culture system comprising modified stem cells or expanded cell preparations from which genes, proteins, and other metabolites involved in proliferation of stem cells, in particular hematopoietic cells, can be identified and isolated. The stem cells in a culture system of the invention may be compared with other cells (e.g. differentiated cells) to determine the mechanisms and compounds that stimulate production of mature cells, in particular hematopoietic cells.

Hematopoietic cells and cell preparations of the invention can be used to prepare a cDNA library relatively uncontaminated with cDNA preferentially expressed in cells from other lineages. Hematopoietic cells and cell preparations of the invention can also be used to prepare antibodies specific for markers of hematopoietic cells according to methods known to a skilled artisan.

The following non-limiting examples are illustrative of the present invention:

### EXAMPLE 1

### Enhanced Ex Vivo Expansion of Hematopoietic Stem Cells by NUP-Hox Fusion Genes

Expanding hematopoietic stem cells (HSCs) remains a considerable challenge and needs an improved strategy. HOXB4 over expression is known to enhance HSC expansion 41-fold in 2-week culture. HOXA9 has also been reported to trigger HSC expansion in vivo prior to onset of myeloproliferative disease. Overexpression of naturally occurring and engineered novel NUP98-Hox fusion genes showed enhanced effects in CFU-S output and blocked differentiation in vitro with NUP98-Abd-B-like HOX genes (NUP98-HOXA10 (NA10) and NUP98-HOXD13 (ND13) more potent than NUP98-Antennepedia-like HOX genes (NUP98-HOXB4 (NB4) and NUP98-HOXB3 (NB3). In order to evaluate overlap or differences in HSC expanding potentials between different prologues of HOX genes, specifically NUP98-Hox-fusions, in vitro HSC expansion of NB4 and NA10 were compared to HOXB4 using a limiting dilution assay for competitive repopulation unit (CRU) for lympho-myeloid reconstitution (>1% of donor-derived cells in PB). 5-FU treated BM cells were harvested (Day 0), and 3×10⁵ cells per culture were prestimulated with IL-3, IL-6, and SK for 2 days, retrovirally transduced with each gene or GFP, and cultured for a further 6 days for a total of 10 days. The transduced cells were transplanted at various dilutions without selection into sublethally irradiated recipients at Day 10. The proportion of GFP positive cells were 77.3-92.2% at the time of transplant. At Day 0 CRU frequency was 1 in 2238 cells or each culture contained 134 CRUs. After 10-day culture there were >34 CRUs in the GFP culture, while there were >150000, 37500, and 16700 CRUs in the NA10, NB4, and HOXB4 culture, respectively. Consequently, total number of CRUs increased by >1100-fold, 280-fold, and 120-fold in the NA10, NB4, and HOXB4 culture, respectively. Similar results were observed in another experiment for NA10 and NB4, and in previous study for HOXB4. These data indicate that the ability of Hox to enhance HSC expansion is not unique to HOXB4 and that an Abd-B-like HOX gene (HOXA10) is more potent than Antennepedia-like-HOX gene (HOXB4) when combined with NUP98. Furthermore, the comparison of NB4 with HOXB4 revealed that HSC expanding potential can be augmented by fusion to a NUP98 gene. Altogether, overexpression of NUP98 fusion genes, in particular NA10, might provide a more powerful strategy to expand HSCs ex vivo. To avoid the leukemogenic effects, however, protein based delivery systems or inducible gene transfer systems are preferred.

### EXAMPLE 2

### Enhanced Repopulation of Sublethally Conditioned Mice Using Ex Vivo Expanded HOXB4-Transduced Hematopoietic Stem Cells (HSC)

Reduced intensity regimens are of interest as a way to minimize morbidity and mortality associated with stem cell transplantation-based therapies. However, achievement of high level donor chimerism in this setting requires the use of additional strategies to allow the transplanted cells to outcompete the large numbers of surviving endogenous HSCs. One approach is to increase the number of HSCs transplanted, as confirmed in studies showing 220% long term donor chimerism following the transplantation of 1.5×10⁶ day 4 5-FU bone marrow (BM) cells into mice given 200 cGY. Based on the ability of forced HOXB4 expression to increase HSCs (>40-fold) within 2 weeks in vitro, the applicability of this approach was investigated in recipients given reduced intensity preparative regimens. 5-FU murine BM was exposed to retroviral vectors encoding HOXB4 and/or GFP, cultured for a further 7 days and the progeny of 8,000 or 80,000 original 5-FU BM cells then transplanted into mice given 250 cGy. At 2 months post-transplant, no donor-derived cells were present in the peripheral ±5% in recipients of the higher transplant dose. Transplantation of the lower dose of HOXB4 infected cells gave equivalent chimerism as the higher dose of GFP-infected cells (12±7%) and 63+12% chimerism in recipients of the higher dose of HOXB4-infected cells. Donor chimerism was lympho-myeloid and stable out to 7 months. These results show that HSC expanded in vitro using HOXB4 retain full lympho-myeloid reconstituting potential in minimally conditioned recipients and importantly may enable reductions in cell dose of some 20-200 fold to achieve clinically useful levels of donor chimerism. This now sets the stage for future applications in gene therapy and other settings where non-myeloablative treatments would be highly desirable.

### EXAMPLE 3

### A NUP98-HOX Fusion Gene Containing Only the Homeodomain of HOXA10 Stimulates Very Large Expansions of Hematopoietic Stem Cells in Culture

Engineered overexpression of the homeobox transcription factor HOXB4 has emerged as a powerful stimulator of hematopoietic stem cell (HSC) expansion in vitro (>40-fold). Strikingly, this activity is augmented by fusion to the N-terminus of NUP98, a gene that fuses with multiple partners in human AML. Studies described herein indicated that remarkable expansions of HSC (>1.000-fold) could be achieved in vitro by forced expression of engineered fusions between NUP98 and the second exon of HOXA10. This second exon of HOXA10 encodes a homeodomain plus flanking sequences of unknown function and a Pbx-binding motif. To analyze further the HOXA10 sequence requirements to achieve the effect obtained on HSC, a novel NUP98-fusion gene has been tested that retains only the homeodomain of HOXA10 (NUP98-HOXA10(hd)) [See SEQ ID NO. 35]. Cultures initiated with 3×106 5-FU pre-treated mouse marrow cells were prestimulated with IL-3, IL-6 and SF, retrovirally-transduced with GFP control or NUP98-HOXA10hd vectors and cultured for another 6 days with the same growth factors. Limiting dilution assays of competitive lympho-myeloid repopulating (>4 months) unit (CRU) frequencies before and after culture showed that the control CRU content had declined ˜50-fold (from 640 to 12) by day 10. In contrast, the CRU content of the cultures of NUP98-HOXA10hd-transduced cells increased (from 500-fold to >2000-fold) and proviral integration analysis of cells from the reconstituted recipients revealed this was a polyclonal expansion of CRU activity. Similarly, the same effect was obtained in cultures of NUP98-HOXA10hd-transduced cells that had been initiated with limiting numbers of input CRU (1-2). These findings confirm the extreme potency of NUP98-HOX fusions as novel agents for HSC expansion, reveal the sufficient contribution of the DNA-binding homeodomain to achieve this effect and set the stage for the design of minimal Hox-based molecules for HSC expansion.

TABLE 1

| | Polypeptide | | | |
|---|---|---|---|---|
| | Accession | | | Gene Accession |
| | Number | | | Number |
| | (NCBI)/SEQ | | | (NCBI/SEQ ID |
| Polypeptide Name | ID NO. | Organism | Gene Symbol | NO. |
| Homeobox Protein | P17483 | Homo sapiens | HOXB4 or HOX2F | AF307160 |
| Hox-B4 (Hox-2F) | AAH49204 | | | BC049204 |
| (Hox-2.6) | SEQ ID NO.5 | | | SEQ ID NO.3 |
| | | | | (second exon) and 4 |
| Homeobox Protein | P10284 | Mus musculus | HOXB4 or | M36654 |
| Hox-B4 | AAH49204 | | HOXB-4 or HOX-2.6 | |
| Homeobox Protein | P31260 | Homo sapiens | HOXA10 | AF040714 |
| Hox-A10 (Hox-1H), | Q15949 | | HOX-1H | X58430 |
| (Hox-1.8) | SEQ ID NO.8 | | | BC071843 |
| | | | | SEQ ID NO.6 and |
| | | | | 7 (Second exon) |
| Homeobox Protein | P31310 | Mus musculus | HOXA10 | L08757 |
| Hox-A10, (Hox-1.8) | | | HOXA-10, | |
| | | | HOX-1.8 | |
| Homeobox Protein | P35453 | Homo sapiens | HOXD13 or HOX4I | NM_000523 |
| Hox-D13, (Hox-41) | SEQ ID NO.10 | | | AB032481 |
| | | | | SEQ ID NO.9 |
| Homeobox Protein | P70217 | Mus musculus | HOXD13 or HOX-4.8 | X99291 |
| Hox-D13, (Hox-41) | Q64177 | | | |
| Homeobox Protein | P31269 | Homo sapiens | HOXA9 or | NM_152739 |
| Hox-A9, (Hox-41) | 099820 | | HOXIG | NM_002142 |
| | 043369 | | | SEQ ID NO.13 and 14 |
| | 043429 | | | |
| | SEQ ID NO.15 | | | |
| Homeobox Protein | P09631 | Mus musculus | HOXA9 or HOXA-9 or | AB005457 |
| Hox-A9, (Hox-1.7) | 070154 | | HOX-1.7 | AB008914 |
| Homeobox Protein | P14651 | Homo sapiens | HOXB3 | NM_002146 |
| Hox-B3 | NP 002137 | | HOX2 | X16667 |
| | SEQ ID NO.12 | | HOX2G | SEQ ID NO.11 |
| | | | HOX-2.7 | |
| Homeobox Protein | NP_034598 | Mus musculus | HOXB3 | |
| Hox-B3 | | | | |

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the host cells, vectors, methodologies etc. which are reported therein which might be used in connection with the invention. Nothing herein is to be construed as an admission that the invention is not entitled to antedate such disclosure by virtue of prior invention.

What is claimed is:

1. A method of expanding a population of stem cells comprising modifying the stem cells with a (i) a sequence encoding a homeodomain polypeptide and a sequence encoding a transcription activation domain (TAD); (ii) a homeodomain polypeptide and a TAD; or (iii) an Abd-B-like HOX polynucleotide or a Abd-B-like polypeptide.

2. A method of enhancing expansion of stem cells comprising delivering to the stem cells an effective amount of (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-B-like HOX polynucleotide or a Abd-B-like polypeptide; to provide enhanced expansion of the stem cells.

3. A method according to claim 1 or 2 wherein the stem cells are cultured ex vivo to thereby expand the stem cells.

4. A method for expanding hematopoietic stem cells and progenitor cells comprising (a) obtaining a sample comprising stem cells and enriching for stem cells by positive or negative selection; (b) modifying the enriched stem cells so that they comprise (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or, (iii) an Abd-B-like HOX polynucleotide or a Abd-B-like polypeptide; and (c) culturing and isolating increased numbers of stem cells and progenitor cells.

5. A method according to claim 4 wherein in (a) the cells are enriched for CD34⁺ cells.

6. A method according to claim 4 which is characterized by not altering the normal proportion of mature blood cells and/or commitment to specific blood lineages obtained with non-modified cells.

7. A method according to any preceding claim wherein modifying the stem cells results in at least a 10-fold, 20-fold, 50-fold, or 1000-fold expansion of pluripotent stem cells relative to expansion of stem cells modified with HOXB4 alone.

8. A method for enhancing the stability and/or potency of HOXB4 for enhancing expansion of stem cells comprising obtaining stem cells and modifying the stem cells with a HOXB4 polynucleotide fused to a sequence encoding a TAD wherein expansion of the stem cells is increased at least 10-fold, 20-fold, 50-fold, or 1000-fold relative to expansion of the stem cells with HOXB4 alone.

9. A method for modifying stem cells comprising obtaining stem cells to be genetically modified, providing the stem cells ex vivo with conditions for cell proliferation, and genetically modifying the stem cells with a homeobox polynucleotide and a sequence encoding a TAD, or delivering a homeodomain polypeptide and a TAD polypeptide into the cells.

10. A method according to any preceding claim wherein the sequence encoding a homeodomain polypeptide is fused to the sequence encoding the TAD to provide a nucleic acid construct.

11. A method according to any preceding claim wherein the homeodomain polypeptide is fused to the TAD to provide a chimeric polypeptide.

12. A method of producing hematopoietic stem cells from embryonic stem cells comprising obtaining embryonic stem cells and modifying the stem cells with (i) a homeodomain polypeptide; (ii) a homeobox polynucleotide; (iii) a sequence encoding a homeodomain polypeptide and a sequence encoding a transcription activation domain (TAD); or (iv) a homeodomain polypeptide and a TAD so that the embryonic stem cells form hematopoietic stem cells.

13. A method for enhancing expansion or output of hematopoietic stem cells from embryonic stem comprising modifying the embryonic stem cells with an effective amount of (i) a homeodomain polypeptide; (ii) a homeobox polynucleotide; (iii) a sequence encoding a homeodomain polypeptide and a sequence encoding a transcription activation domain (TAD); or (iv) a homeodomain polypeptide and a TAD, to generate expanded populations of hematopoietic stem cells.

14. A method according to claim 12 or 13 wherein the embryonic stem cells are initiated embryonic stem cells.

15. A method according claim 12 or 13 wherein the embryonic stem cells are differentiated embryonic stem cells.

16. A method according to any preceding claim further comprising administering the modified or expanded stem cells to a subject to reconstitute stem cells in the subject.

17. A method according to any preceding claim wherein the cells are hematopoietic cells.

18. A method according to any one of claims 1, 2, 3, and 7 through 11 wherein the cells are embryonic stem cells.

19. An isolated cell preparation comprising modified stem cells produced by a method according to any one of claims 1 and 4 through 8.

20. An ex vivo expanded cell preparation obtained by a method of any preceding claim.

21. A stem cell modified to express a nucleic acid construct comprising a homeobox polynucleotide and a sequence encoding a TAD, wherein the stem cell has an enhanced ability to proliferate to form an expanded population of pluripotent stem cells.

22. A stem cell according to claim 21 which has an at least 10-fold, 20-fold, 50-fold, or 1000-fold enhanced ability to proliferate relative to a stem cell modified with HOXB4 alone.

23. A method for treating a condition or disease in a subject in which reconstitution of stem cells is desirable comprising administering a therapeutically effective amount of stem cells modified with (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD and optionally an element that enhances or facilitates delivery of the sequences into stem cells; or (iii) an Abd-B-like HOX polynucleotide or a Abd-B-like polypeptide.

24. A method for restoring hematopoietic capability to a mammalian subject comprising the steps of:
a) removing hematopoietic stern cells from a mammalian subject;
b) modifying the stem cells with (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitated delivery of the sequences into stem cells; or (iii) an Abd-B-like HOX polynucleotide or a Abd-B-like polypeptide
c) expanding the stem cells to form an expanded population of stem cells; and
d) returning the expanded stem cells to the subject, wherein hematopoietic capability is restored to the subject.

25. A method of claim 23 or 24 wherein the stem cells are hematopoietic cells.

26. A method according to claim 23, 24, or 25 wherein the subject has a condition or disease involving hematopoietic cells.

27. A method of claim 23, 24, 25 or 26 wherein the stem cells are administered to a subject and expanded in vivo.

28. A method of claim 23, 24, 25, or 26 wherein the stem cells are expanded in vitro and administered to the subject.

29. A method according to any preceding claim further comprising modifying the stem cells with a proliferation factor.

30. A method of hematopoietic cell transplantation comprising obtaining hematopoietic stem cells to be transplanted from a donor; modifying the hematopoietic stem cells with (i) a sequence encoding a homeodomain polypeptide and a sequence encoding a transcription activation domain (TAD); (ii) a homeodomain polypeptide and a TAD; or (iii) an Abd-B-like HOX polynucleotide or a Abd-B-like polypeptide; culturing the hematopoietic stem cells under proliferation conditions to thereby expand the hematopoietic stem cells; and transplanting the hematopoietic stem cells to a patient.

31. A method of claim 30 wherein the donor and patient is a single individual.

32. A method of adoptive immunotherapy comprising obtaining hematopoietic stem cells from a patient; modifying the hematopoietic stem cells with (i) a sequence encoding a homeodomain polypeptide and a sequence encoding a transcription activation domain (TAD); (ii) a homeodomain polypeptide and a TAD; or (iii) an Abd-B-like HOX polynucleotide or a Abd-B-like polypeptide; culturing the hematopoietic stem cells under proliferation conditions to thereby expand the hematopoietic stem cells; and transplanting the hematopoietic stem cells to the patient.

33. A method for restoring hematopoietic capability to a human subject comprising recovering stem cells from the subject, modifying and expanding the stem cells in vitro wherein the stem cells are modified with (i) a sequence encoding a homeodomain polypeptide and a sequence encoding a transcription activation domain (TAD) and optionally a sequence encoding an element that enhances or facilitates delivery of the sequences into stem cells; (ii) a homeodomain polypeptide and a TAD, and optionally an element that enhances or facilitated delivery of the sequences into stem cells; or (iii) an Abd-B-like HOX polynucleotide or a Abd-B-like polypeptide, and returning the stem cells to the subject or a different subject, resulting in enhancement or restoration of hematopoietic capability to the subject.

34. A method of claim 33 wherein the stem cells are hematopoietic stem cells or embryonic stem cells.

35. A method for preventing and/or treating leukemia in a patient comprising administering to the patient a therapeutically effective amount of stem cells modified with (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-B-like HOX polynucleotide or a Abd-B-like polypeptide.

36. A gene therapy method comprises removing hematopoietic stem cells from a subject, transducing the stem cells in vitro with an exogenous gene and a sequence encoding a homeodomain polypeptide and a sequence encoding a transcription activation domain (TAD); or an Abd-B-like HOX polynucleotide, and administering transduced stem cells to a subject wherein the stem cells having the capability of substantial self-renewal and ability to give rise to all hematopoietic cell lineages.

37. A method of claim 36 wherein the exogenous gene encodes a therapeutic.

38. An in vivo method for expanding stem cells in a subject in need thereof comprising administering to stem cells in the subject a therapeutically effective amount of (i) a sequence encoding a homeodomain polypeptide and a sequence encoding a transcription activation domain (TAD) and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-B-like HOX polynucleotide or a Abd-B-like polypeptide to thereby in vivo expand the stem cells in the subject.

39. A method of claim 38 wherein the stem cells of the subject are transduced in vivo with a homeodomain polypeptide and nucleoporin.

40. A method of claim 38 wherein stem cells of the subject are transduced in vivo with a vector(s) comprising a nucleic acid construct, a homeobox polynucleotide and a sequence encoding a TAD, or an Abd-B-like HOX polynucleotide, and an inducible regulatory element which is activated by an endogenous or exogenous factor.

41. A method according to any one of claims 23 to 40 wherein the sequence encoding a homeodomain polypeptide is fused to the sequence encoding the TAD to provide a nucleic acid construct.

42. A method according to any one of claims 23 to 40 wherein the homeodomain polypeptide is fused to the TAD to provide a chimeric polypeptide.

43. An isolated nucleic acid construct as defined in any preceding claim.

44. An isolated nucleic acid construct comprising a homeobox polynucleotide and a sequence encoding a TAD, and optionally a sequence encoding an element that enhances or facilitates delivery of the sequences into stem cells wherein the construct provides enhanced expansion of stem cells.

45. An isolated nucleic acid construct of claim 44 wherein the homeobox polynucleotide is an Abd-B-like HOX polynucleotide.

46. An isolated nucleic acid construct of claim 44 wherein the homeobox polynucleotide is HOXA10, HOXD13, HOXB4, or HOXB3.

47. An isolated nucleic acid construct of any preceding claim wherein the TAD is a nucleoporin.

48. An isolated nucleic acid construct of any preceding claim wherein the TAD is Nup98.

49. An isolated nucleic acid construct of any preceding claim comprising a sequence of SEQ ID NO. 16, 18, 20, 22, 24, or 25.

50. Use of (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-3-like HOX polynucleotide or a Abd-B-like polypeptide, in the preparation of a medicament for restoring hematopoietic capability to a mammalian subject.

51. Use of a therapeutically effective amount of stem cells modified with (i) a sequence encoding a homeodomain polypeptide and a sequence encoding a transcription activation domain (TAD); (ii) a homeodomain polypeptide and a TAD; or (iii) an Abd-B-like HOX polynucleotide or a Abd-B-like polypeptide, in the preparation of a medicament for treating a condition or disease in a subject in which reconstitution of stem cells is desirable.

52. Use of a therapeutically effective amount of hematopoietic stem cells modified with (i) a sequence encoding a homeodomain polypeptide and a sequence encoding a transcription activation domain (TAD); (ii) a homeodomain polypeptide and a TAD; or (iii) an Abd-B-like HOX polynucleotide or a Abd-B-like polypeptide, in the preparation of a medicament for restoring hematopoietic capability to a mammalian subject.

53. Use of claim 51 or 52 wherein the subject has a condition or disease involving hematopoietic cells.

54. Use of a therapeutically effective amount of stem cells modified with (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-B-like HOX polynucleotide or a Abd-B-like polypeptide in the preparation, of a medicament for transplantation into a subject in need of such transplantation.

55. Use of a therapeutically effective amount of stem cells modified with (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-B-like HOX polynucleotide or a Abd-B-like polypeptide, in the preparation of a medicament for adoptive immunotherapy.

56. Use of a therapeutically effective amount of hematopoietic stem cells modified with (i) a sequence encoding a homeodomain polypeptide and a sequence encoding a transcription activation domain (TAD), and optionally a sequence encoding an element that enhances or facilitates delivery of the sequences into stem cells; (ii) a homeodomain polypeptide and a TAD, and optionally an element that enhances or facilitates delivery of the sequences into stem cells; or (iii) an Abd-B-like HOX polynucleotide or a Abd-B-like polypeptide, in the preparation of a medicament for preventing and/or treating leukemia in a subject.

57. Use of a therapeutically effective amount of stem cells modified with a sequence encoding a exogenous gene and a sequence encoding a homeodomain polypeptide and a sequence encoding a transcription activation domain (TAD), or an Abd-B-like HOX polynucleotide, in the preparation of a medicament for gene therapy.

58. Use according to claim 57 wherein the exogenous gene encodes a therapeutic.

59. A kit for carrying out a method according to any preceding claim.


SEQUENCE LISTING (38 sequences)

SEQ ID NO: 1 - DNA, 1410 bp, Artificial Sequence
  NUP98 portion shared among engineered NUP98- Hox fusion genes NUP98 (ATG removed)
tttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagaattc

SEQ ID NO: 2 - protein, 470 aa, Artificial Sequence
  Amino acid Sequence Nup98 in fusions EF aa generated from synthetic EcoRI fusion site
FNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVEF

SEQ ID NO: 3 - DNA, 306 bp, Artificial Sequence
  HOXB4:(NCBI: AF307160) Fusion #1 NB4: fusion of whole second exon of B4 (EcoRI site added upstream start of B4 second exon
gaattcgtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctag

SEQ ID NO: 4 - DNA, 2040 bp, Homo sapiens
  ref <308>: DATABASE ACCESSION NUMBER: BC049204
  ref <309>: DATABASE ENTRY DATE: 2004-06-30
  ref <313>: RELEVANT RESIDUES IN SEQ ID NO: (1)..(2040)
ggaaaacgagtcaggggtcggaataaattttagtatattttgtgggcaattcccagaaattaatggctatgagttcttttttgatcaactcaaactatgtcgaccccaagttccctccatgcgaggaatattcacagagcgattacctacccagcgaccactcgcccgggtactacgccggcggccagaggcgagagagcagcttccagccggaggcgggcttcgggcggcgcgcggcgtgcaccgtgcagcgctacgcggcctgccgggaccctgggcccccgccgcctccgccaccacccccgccgcccccgccaccgcccggtctgtcccctcgggctcctgcgccgccacccgccggggccctcctcccggagcccggccagcgctgcgaggcggtcagcagcagccccccgccgcctccctgcgcccagaaccccctgcaccccagcccgtcccactccgcgtgcaaagagcccgtcgtctacccctggatgcgcaaagttcacgtgagcacggtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctagtgcccccgcacgcgggagccacgaacctcggggtgggggtgggcagtgagtgcaggggatggggtggggggacaggagggggccctggggcctgggccccggaaaaatctatctgccctcccccacactttatatacgaataaacgcagaagagggggaggggaagctttatttatagaaatgacaatagagggccacggggaggcccccccagaagcaagattcaaatctcttgctttctttcttaaaaaaaagaaaaagaaaaagcaagaagaaggaagaaagaaaaagacagaaagagaaataggaggaggctgcagctcctcgttttcagctttggcgaagatggatccacgtttcatctttaatcacgccaggtccaggcccatctgtcttgtttcctctgccgaggagaagacgggcctcggtggcgaccattacctcgacacccgctaacaaatgaggcccggctcggccgcctccgcctctgctactgccgctgctggaagacagcctggatttcctttctttgtcccccactcccgatacccagcgaaagcaccctctgactgccagatagtgcagtgttttggtcacggtaacacacacacactctccctcatctttcgtgcccattcactgagggccagaatgactgctcacccacttccaccgtggggttgggggtgggcaacagaggaggggagcaagtagggaagggggtggccttgacaactcaggagtgagcagggaaattgagtccaaggaaaaagagagactcagagacccgggagggccttcctctgaaaggccaagccaagccatgcttggcagggtgaggggccagttgagttctgggagctgggcactactctgccagtccagagttgtacagcagaagcctctctcctagactgaaaatgaatgtgaaactaggaaataaaatgtgcccctcccagtctgggaggaggatgttgcagagccctctcccatagtttattatgttgcatcgtttattattattattgataatattattattactatttttttgtgtcatgtgagtcctctctccttttctctttctgacattccaaaaccaggccccttcctacctctggggctgcttgagtctagaacccttcgtatgtgtgaatatctgtgtgctgtacagagtgacaatagaaataaatgtttggtttcttgtgaaaaaaaaaaaaaa

SEQ ID NO: 5 - protein, 251 aa, Homo sapiens
  ref <308>: DATABASE ACCESSION NUMBER: AAH49204
  ref <309>: DATABASE ENTRY DATE: 2004-06-30
  ref <313>: RELEVANT RESIDUES IN SEQ ID NO: (1)..(251)
MAMSSFLINSNYVDPKFPPCEEYSQSDYLPSDHSPGYYAGGQRRESSFQPEAGFGRRAACTVQRYAACRDPGPPPPPPPPPPPPPPPGLSPRAPAPPPAGALLPEPGQRCEAVSSSPPPPPCAQNPLHPSPSHSACKEPVVYPWMRKVHVSTVNPNYAGGEPKRSRTAYTRQQVLELEKEFHYNRYLTRRRRVEIAHALCLSERQIKIWFQNRRMKWKKDHKLPNTKIRSGGAAGSAGGPPGRPNGGPRAL

SEQ ID NO: 6 - DNA, 1183 bp, Artificial Sequence
  HOXA10 Nucleic acid sequence  AF040714 (gi|2789671:320-1226,2401-2675)  Homo sapiens homeobox protein A10 (HOXA10) gene, complete cds
atgtcatgctcggagagccccgccgcgaactcttttttggtcgactcgctcatcagctcgggcagaggcgaggcaggcggcggtggtggtggcgcggggggcggcggcggtggcggttactacgcccacggcggggtctacctgccgcccgccgccgacctgccatacgggctgcagagctgcgggctcttccccacgctgggcggcaagcgcaatgaggcagcgtcgccgggcagcggtggcggtggcgggggtctaggtcccggggcgcacggctacgggccctcgcccatagacctgtggctagacgcgccccggtcttgccggatggagccgcctgacgggccgccgccgccgccccagcagcagccgccgcccccgccgcaaccaccccagccagcgccgcaggccacctcgtgctctttcgcgcagaacatcaaagaagagagctcctactgcctctacgactcggcggacaaatgccccaaagtctcggccaccgccgccgaactggctcccttcccgcggggcccgccgcccgacggctgcgccctgggcacctccagcggggtgccagtgcctggctacttccgcctttctcaggcctacggcaccgccaagggctatggcagcggcggcggcggcgcgcagcaactcggggctggcccgttccccgcgcagcccccggggcgcggtttcgatctcccgcccgcgctagcctccggctcggccgatgcggcccggaaggagcgagccctcgattcgccgccgccccccacgctggcttgcggcagcggcgggggctcgcagggcgacgaggaggcgcacgcgtcgtcctcggccgcggaggagctctccccggccccttccgagagcagcaaagcctcgccggagaaggattccctgggcaattccaaaggtgaaaacgcagccaactggctcacggcaaagagtggtcggaagaagcgctgcccctacacgaagcaccagacactggagctggagaaggagtttctgttcaatatgtaccttactcgagagcggcgcctagagattagccgcagcgtccacctcacggacagacaagtgaaaatctggtttcagaaccgcaggatgaaactgaagaaaatgaatcgagaaaaccggatccgggagctcacagccaactttaatttttcctgac

SEQ ID NO: 7 - DNA, 282 bp, Artificial Sequence
  NA10: second exon of A10
gaattcggcaattccaaaggtgaaaacgcagccaactggctcacggcaaagagtggtcggaagaagcgctgcccctacacgaagcaccagacactggagctggagaaggagtttctgttcaatatgtaccttactcgagagcggcgcctagagattagccgcagcgtccacctcacggacagacaagtgaaaatctggtttcagaaccgcaggatgaaactgaagaaaatgaatcgagaaaaccggatccgggagctcacagccaactttaatttttcctga

SEQ ID NO: 8 - protein, 393 aa, Homo sapiens
  ref <308>: DATABASE ACCESSION NUMBER: P31260
  ref <309>: DATABASE ENTRY DATE: 2005-05-01
  ref <313>: RELEVANT RESIDUES IN SEQ ID NO: (1)..(393)
MSCSESPAANSFLVDSLISSGRGEAGGGGGGAGGGGGGGYYAHGGVYLPPAADLPYGLQSCGLFPTLGGKRNEAASPGSGGGGGGLGPGAHGYGPSPIDLWLDAPRSCRMEPPDGPPPPPQQQPPPPPQPPQPAPQATSCSFAQNIKEESSYCLYDSADKCPKVSATAAELAPFPRGPPPDGCALGTSSGVPVPGYFRLSQAYGTAKGYGSGGGGAQQLGAGPFPAQPPGRGFDLPPALASGSADAARKERALDSPPPPTLACGSGGGSQGDEEAHASSSAAEELSPAPSESSKASPEKDSLGNSKGENAANWLTAKSGRKKRCPYTKHQTLELEKEFLFNMYLTRERRLEISRSVHLTDRQVKIWFQNRRMKLKKMNRENRIRELTANFNFS

SEQ ID NO: 9 - DNA, 1461 bp, Homo sapiens
  ref <308>: DATABASE ACCESSION NUMBER: NM_000523
  ref <309>: DATABASE ENTRY DATE: 2005-04-22
  ref <313>: RELEVANT RESIDUES IN SEQ ID NO: (1)..(1461)
gagaaaggagaggagggaggaggcgcgccgcgccatggtgtcctgcgcggggccagggccagggccggggccgggccaggccgggccatgagccgcgccgggagctgggacatggacgggctgcgggcagacggcgggggcgccggtggcgccccggcctcttcctcctcctcatcggtggcggcggcggcggcgtcaggccagtgccgcggctttctctccgcgcctgtgttcgccgggacgcattcggggcgggcggcggcggcggcagcggcggctgcggcggcggcggcggcagcctccggctttgcgtaccccgggacctctgagcgcacgggctcttcctcgtcgtcgtcctcttctgccgttgtagcggcgcgcccggaggctcccccagccaaagagtgcccagcacccacgcctgcagcggccgctgcagcgcccccgagcgctccagcgctgggctacggctaccacttcggcaacggctactacagctgccgtatgtcgcacggcgtgggcttacagcagaatgcgctcaagtcatcgccgcacgcctcgctgggaggctttcccgtggagaagtacatggacgtgtcaggcctggcgagcagcagcgtaccggccaacgaggtgccagcgcgagccaaggaggtatccttctaccagggctatacgagcccttaccagcacgtgcccggctatatcgacatggtgtccactttcggctccggggagcctcggcacgaggcctacatctccatggaggggtaccagtcctggacgctggctaacgggtggaacagccaggtgtactgcaccaaggaccagccacaggggtcccacttttggaaatcttcctttccaggggatgtggctctaaatcagccggacatgtgcgtctaccgaagagggaggaagaagagagtgccttacaccaaactgcagcttaaagaactggagaacgagtatgccattaacaaattcattaacaaggacaagcggcggcgtatctcggctgctacgaacctatctgagagacaagtgaccatttggtttcagaaccgaagagtgaaggacaagaaaattgtctccaagctcaaagatactgtctcctgatgtggtccaggttggccacagacagcttagaagccattcggttgtctccaaaaggcctttggaaagacttgaatatgtatttaattccccccaccccctgccaatggtggcaaattttgtgaattgtttttctctcttccccttatctggctctaaaaccttctgctgcccaacctgactttgtagttctgatttttacttgtttattattggttttgttcttgcctagggtttttaaaatatctgtttttaatgttttgtttctccctccaggccagtataaagggacttgaagtattttttaataatccgccccccaatgaacttcagaagtgccattct

SEQ ID NO: 10 - protein, 335 aa, Homo sapiens [sequence omitted]

SEQ ID NO: 11 - DNA, 2603 bp, Homo sapiens
  ref <308>: DATABASE ACCESSION NUMBER: NM_002146
  ref <309>: DATABASE ENTRY DATE: 2005-04-23
  ref <313>: RELEVANT RESIDUES IN SEQ ID NO: (1)..(2603)
ctgggtagggcagggggaaccgacaggccggtgtccccagccgcaaaagagctgctgaactgtccgtttaaatgctgctgggagactcgtaaaaaaatcatcgtggacctggaggatgagaggggcgagctttatttcggtcggattgcggtgtggtggtttagctgcaaggggatgccgcagccccagttgagggggaaaatagttcttaaaaagcatatgcccccctaaggaatgtctctaaagaaccaaatcaaagctgctctttggaaggtatgaatagaatttaaaaaaaaaagatttctatggagcttaaagttcacagccattctgtgtagacaagagctaagaaaaatgtgagaattatacagaaaaccattaatcacttcttttctttaaatacgtatcctctctcctttgttattattcaacagcaaatctccttggaccggctgttgggggaaaaaagtgttagccgtctctcccggatctgcaagggggaaaaaatttggaaccataaagttgaaaacttttttctctcagtttggaagaagcccttcgtcatgaatgggatctgcagagttcgggcgagaggaggcgagaggcgcaaaggaggggagatttgtcgcctgccgctcgctctggggctcgatgtgaatatatattatgtctgcctgttctcccctcgtcggtggctaaggtcagccgcttggaacagaccccggaggaggggggcagagaggggaggtgggggggggggggtccggcgtgtcacgtgacccccagggttgccaatgtccggtcctgagggtatcaggcctttccaagttgccacccactgcccaggcctcacccagcgatgcagaaagccacctactacgacaacgccgcggctgctctcttcggaggctattcctcgtaccctggcagcaatggcttcggcttcgatgtccccccccaacccccatttcaggccgccacgcacctggagggcgactaccagcgctcagcttgctcgctgcagtccctgggcaacgctgccccacatgccaagagcaaggagctcaacggcagctgcatgaggccgggtctggcccccgagaccctgtcggccccgcctggctcacccccgcccagtgccgcacctaccagtgccactagcaacagcagtaatgggggcgggcccagcaaaagtggtcccccaaagtgcggtcccggcaccaactccaccctcaccaaacagatattcccctggatgaaagagtcgaggcaaacgtccaagctgaaaaacaactcccccggcacagcagagggctgtggtggcggcggcggtggcggcggcggcggaggcagtggtggcagcgggggcggtggcggcggcggcgggggaggggacaagagccccccggggtcggcggcgtccaagcgggcgcggacggcgtacacgagcgcgcagctggtggagctggagaaggagttccattttaaccgctacctgtgccggcctcgccgtgtagagatggccaacctgctgaacctcagcgagcggcagatcaagatctggttccagaaccggcgcatgaagtacaagaaggaccagaaggccaagggattggcctcgtcgtcggggggcccatctccagccggcagccccccgcagcccatgcagtccacggccggcttcatgaacgccttacactccatgacccccagctacgagagcccgtccccacccgccttcggtaaagcccaccagaatgcctacgcgctgccctccaactaccagccccctctcaaaggctgcggcgccccgcagaagtaccctccgaccccggcgcccgagtatgagccgcacgtcctccaagccaacgggggcgcctacgggacgcccaccatgcagggcagtccggtgtacgtgggcgggggcggctacgcggatccgctgccgccccctgccggcccctccctctatggcctcaaccacctttcccatcacccttccgggaacctggactacaacggggcgccccctatggcgcccagccagcaccacggaccctgcgaaccccaccccacctacacagacctctcctctcaccacgcgcctcctcctcagggtagaatccaagaagcgcccaaattaacacacctgtgatgggaaagggcgaacgaggattaggggatggggaggaagagagagactgtggagctctggggggcaacctggaggtctgaaaagaggagccagagaaggtggtacccaggcttcctggtcagaaccggcctggagctccttcccttccccctggcctgagaggttgcttttaagtcttccaccccttgttccatctgcctgccaacccatcggaaaggaatccacatcatattggagatgaccccatcaaccccagggctccagcactaccaagttggaattccacgcccgggagtggggtagaggaagacgagacaggacgaggcagaaaagcacattttaaaaaccagacaagatggctaggccatcaccaaccaacggacttaccttacatctttgtaggtaattccccccaaatcttgattttttttttcctcaattatccttt

SEQ ID NO: 12 - protein, 431 aa, Homo sapiens
  ref <308>: DATABASE ACCESSION NUMBER: P14651
  ref <309>: DATABASE ENTRY DATE: 2005-05-01
  ref <313>: RELEVANT RESIDUES IN SEQ ID NO: (1)..(431)
MQKATYYDNAAAALFGGYSSYPGSNGFGFDVPPQPPFQAATHLEGDYQRSACSLQSLGNAAPHAKSKELNGSCMRPGLAPETLSAPPGSPPPSAAPTSATSNSSNGGGPSKSGPPKCGPGTNSTLTKQIFPWMKESRQTSKLKNNSPGTAEGCGGGGGGGGGGGSGGSGGGGGGGGGGDKSPPGSAASKRARTAYTSAQLVELEKEFHFNRYLCRPRRVEMANLLNLSERQIKIWFQNRRMKYKKDQKAKGLASSSGGPSPAGSPPQPMQSTAGFMNALHSMTPSYESPSPPAFGKAHQNAYALPSNYQPPLKGCGAPQKYPPTPAPEYEPHVLQANGGAYGTPTMQGSPVYVGGGGYADPLPPPAGPSLYGLNHLSHHPSGNLDYNGAPPMAPSQHHGPCEPHPTYTDLSSHHAPPPQGRIQEAPKLTHL

SEQ ID NO: 13 - DNA, 2076 bp, Homo sapiens
  ref <308>: DATABASE ACCESSION NUMBER: NM_152739
  ref <309>: DATABASE ENTRY DATE: 2005-04-23
  ref <313>: RELEVANT RESIDUES IN SEQ ID NO: (1)..(2076)
agttgttacatgaaatctgcagtttcataatttccgtgggtcgggccgggcgggccaggcgctgggcacggtgatggccaccactggggccctgggcaactactacgtggactcgttcctgctgggcgccgacgccgcggatgagctgagcgttggccgctatgcgccggggaccctgggccagcctccccggcaggcggcgacgctggccgagcaccccgacttcagcccgtgcagcttccagtccaaggcgacggtgtttggcgcctcgtggaacccagtgcacgcggcgggcgccaacgctgtacccgctgcggtgtaccaccaccatcaccaccacccctacgtgcacccccaggcgcccgtggcggcggcggcgccggacggcaggtacatgcgctcctggctggagcccacgcccggtgcgctctccttcgcgggcttgccctccagccggccttatggcattaaacctgaaccgctgtcggccagaaggggtgactgtcccacgcttgacactcacactttgtccctgactgactatgcttgtggttctcctccagttgatagagaaaaacaacccagcgaaggcgccttctctgaaaacaatgctgagaatgagagcggcggagacaagccccccatcgatcccaataacccagcagccaactggcttcatgcgcgctccactcggaaaaagcggtgcccctatacaaaacaccagaccctggaactggagaaagagtttctgttcaacatgtacctcaccagggaccgcaggtacgaggtggctcgactgctcaacctcaccgagaggcaggtcaagatctggttccagaaccgcaggatgaaaatgaagaaaatcaacaaagaccgagcaaaagacgagtgatgccatttgggcttatttagaaaaaagggtaagctagagagaaaaagaaagaactgtccgtcccccttccgccttctcccttttctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgccaaataaaatatattgattcttttctaaaaaaaaaaaaaaaaaaaa

SEQ ID NO: 14 - DNA, 1903 bp, Homo sapiens
  ref <308>: DATABASE ACCESSION NUMBER: NM_02142
  ref <309>: DATABASE ENTRY DATE: 2005-04-23
  ref <313>: RELEVANT RESIDUES IN SEQ ID NO: (1)..(1903)
agttgttacatgaaatctgcagtttcataatttccgtgggtcgggccgggcgggccaggcgctgggcacggtgatggccaccactggggccctgggcaactactacgtggactcgttcctgctgggcgccgacgccgcggatgagctgagcgttggccgctatgcgccggggaccctgggccagcctccccggcaggcggcgacgctggccgagcaccccgacttcagcccgtgcagcttccagtccaaggcgacggtgtttggcgcctcgtggaacccagtgcacgcggcgggcgccaacgctgtacccgctgcggtgtaccaccaccatcaccaccacccctacgtgcacccccaggcgcccgtggcggcggcggcgccggacggcagttgatagagaaaaacaacccagcgaaggcgccttctctgaaaacaatgctgagaatgagagcggcggagacaagccccccatcgatcccaataacccagcagccaactggcttcatgcgcgctccactcggaaaaagcggtgcccctatacaaaacaccagaccctggaactggagaaagagtttctgttcaacatgtacctcaccagggaccgcaggtacgaggtggctcgactgctcaacctcaccgagaggcaggtcaagatctggttccagaaccgcaggatgaaaatgaagaaaatcaacaaagaccgagcaaaagacgagtgatgccatttgggcttatttagaaaaaagggtaagctagagagaaaaagaaagaactgtccgtcccccttccgccttctcccttttctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgccaaataaaatatattgattcttttctaaaaaaaaaaaaaaaaaaaa

SEQ ID NO: 15 - protein, 272 aa, Homo sapiens
  ref <308>: DATABASE ACCESSION NUMBER: P31269
  ref <309>: DATABASE ENTRY DATE: 2005-05-01
  ref <313>: RELEVANT RESIDUES IN SEQ ID NO: (1)..(272)
MATTGALGNYYVDSFLLGADAADELSVGRYAPGTLGQPPRQAATLAEHPDFSPCSFQSKATVFGASWNPVHAAGANAVPAAVYHHHHHHPYVHPQAPVAAAAPDGRYMRSWLEPTPGALSFAGLPSSRPYGIKPEPLSARRGDCPTLDTHTLSLTDYACGSPPVDREKQPSEGAFSENNAENESGGDKPPIDPNNPAANWLHARSTRKKRCPYTKHQTLELEKEFLFNMYLTRDRRYEVARLLNLTERQVKIWFQNRRMKMKKINKDRAKDE

SEQ ID NO: 16 - DNA, 1686 bp, Artificial Sequence
  NA10-PIM sequence excluding FLAG
tttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagaattcggcaattccaaaggtgaaaacgcagccaactggctcacggcaaagagtggtcggaagaagcgctgcccctacacgaagcaccagacactggagctggagaaggagtttctgttcaatatgtaccttactcgagagcggcgcctagagattagccgcagcgtccacctcacggacagacaagtgaaaatctggtttcagaaccgcaggatgaaactgaagaaaatgaatcgagaaaaccggatccgggagctcacagccaactttaatttttcctga

SEQ ID NO: 17 - protein, 561 aa, Artificial Sequence
  AA Sequence NA10
FNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVEFGNSKGENAANWLTAKSGRKKRCPYTKHQTLELEKEFLFNMYLTRERRLEISRSVHLTDRQVKIWFQNRRMKLKKMNRENRIRELTANFNFS

SEQ ID NO: 18 - DNA, 1715 bp, Artificial Sequence
  NUP98-HOXB4 Nucleic acid sequence Sequence of NB4 excluding FLAG sequence
tttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagaattcgtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctagcggcc

SEQ ID NO: 19 - protein, 569 aa, Artificial Sequence
  NUP98-HOXB4 Amino acid sequence
FNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVEFVNPNYAGGEPKRSRTAYTRQQVLELEKEFHYNRYLTRRRRVEIAHALCLSERQIKIWFQNRRMKWKKDHKLPNTKIRSGGAAGSAGGPPGRPNGGPRAL

SEQ ID NO: 20 - DNA, 1684 bp, Artificial Sequence
  NUP98-HOXD13 Nucleic acid sequenceNUP98-HoxD13 (ATG removed)
tttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtaggggatgtggctctaaatcagccggacatgtgcgtctaccgaagagggaggaagaagagagtgccttacaccaaactgcagcttaaagaactggagaacgagtatgccattaacaaattcattaacaaggacaagcggcggcgtatctcggctgctacgaacctatctgagagacaagtgaccatttggtttcagaaccgaagagtgaaggacaagaaaattgtctccaagctcaaagatactgtctcctgatgtggtccaggttggccacagacagctt

SEQ ID NO: 21 - protein, 548 aa, Artificial Sequence
  NUP98-HOXD13 Amino acid sequenceAA Sequence ND13 Fusion
FNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVALNQPDMCVYRRGRKKRVPYTKLQLKELENEYAINKFINKDKRRRISAATNLSERQVTIWFQNRRVKDKKIVSKLKDTVS

SEQ ID NO: 22 - DNA, 2259 bp, Artificial Sequence
  NUP98-HOXB3 Nucleic acid sequence Homeobox containing portion fused (last exon) B3 portion fused through EcoRI siteNB3 sequence excluding FLAG:
tttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagaattcgcagagggctgtggtggcggcggcggtggcggcggcggcggaggcagtggtggcagcgggggcggtggcggcggcggcgggggaggggacaagagccccccggggtcggcggcgtccaagcgggcgcggacggcgtacacgagcgcgcagctggtggagctggagaaggagttccattttaaccgctacctgtgccggcctcgccgtgtagagatggccaacctgctgaacctcagcgagcggcagatcaagatctggttccagaaccggcgcatgaagtacaagaaggaccagaaggccaagggattggcctcgtcgtcggggggcccatctccagccggcagccccccgcagcccatgcagtccacggccggcttcatgaacgccttacactccatgacccccagctacgagagcccgtccccacccgccttcggtaaagcccaccagaatgcctacgcgctgccctccaactaccagccccctctcaaaggctgcggcgccccgcagaagtaccctccgaccccggcgcccgagtatgagccgcacgtcctccaagccaacgggggcgcctacgggacgcccaccatgcagggcagtccggtgtacgtgggcgggggcggctacgcggatccgctgccgccccctgccggcccctccctctatggcctcaaccacctttcccatcacccttccgggaacctggactacaacggggcgccccctatggcgcccagccagcaccacggaccctgcgaaccccaccccacctacacagacctctcctctcaccacgcgcctcctcctcagggtagaatccaagaagcgcccaaattaacacacctgtga

SEQ ID NO: 23 - protein, 752 aa, Artificial Sequence
  NUP98-HOXB3 Amino acid sequenceAA Sequence NB3
FNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVEFAEGCGGGGGGGGGGGSGGSGGGGGGGGGGDKSPPGSAASKRARTAYTSAQLVELEKEFHFNRYLCRPRRVEMANLLNLSERQIKIWFQNRRMKYKKDQKAKGLASSSGGPSPAGSPPQPMQSTAGFMNALHSMTPSYESPSPPAFGKAHQNAYALPSNYQPPLKGCGAPQKYPPTPAPEYEPHVLQANGGAYGTPTMQGSPVYVGGGGYADPLPPPAGPSLYGLNHLSHHPSGNLDYNGAPPMAPSQHHGPCEPHPTYTDLSSHHAPPPQGRIQEAPKLTHL

SEQ ID NO: 24 - DNA, 384 bp, Artificial Sequence
  Fusion:  NB4-PIM: Includes the Pbx Interacting Motif (PIM) of B4-starts 14 codons upstream the PIM motif. engineered EcoRI site shown in bold
gaattcctgcaccccagcccgtcccactccgcgtgcaaagagcccgtcgtctacccctggatgcgcaaagttcacgtgagcacggtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctag

SEQ ID NO: 25 - DNA, 1793 bp, Artificial Sequence
  NB4-PIM sequence excluding FLAG
tttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagaattcctgcaccccagcccgtcccactccgcgtgcaaagagcccgtcgtctacccctggatgcgcaaagttcacgtgagcacggtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctagcggcc

SEQ ID NO: 26 - protein, 3224 aa, Homo sapiens
  ref <308>: DATABASE ACCESSION NUMBER: NP_006267
  ref <309>: DATABASE ENTRY DATE: 2005-04-22
  ref <313>: RELEVANT RESIDUES IN SEQ ID NO: (1)..(3224)
MRRSKADVERYIASVQGSTPSPRQKSMKGFYFAKLYYEAKEYDLAKKYICTYINVQERDPKAHRFLGLLYELEENTDKAVECYRRSVELNPTQKDLVLKIAELLCKNDVTDGRAKYWLERAAKLFPGSPAIYKLKEQLLDCEGEDGWNKLFDLIQSELYVRPDDVHVNIRLVEVYRSTKRLKDAVAHCHEAERNIALRSSLEWNSCVVQTLKEYLESLQCLESDKSDWRATNTDLLLAYANLMLLTLSTRDVQESRELLQSFDSALQSVKSLGGNDELSATFLEMKGHFYMHAGSLLLKMGQHSSNVQWRALSELAALCYLIAFQVPRPKIKLIKGEAGQNLLEMMACDRLSQSGHMLLNLSRGKQDFLKEIVETFANKSGQSALYDALFSSQSPKDTSFLGSDDIGNIDVREPELEDLTRYDVGAIRAHNGSLQHLTWLGLQWNSLPALPGIRKWLKQLFHHLPHETSRLETNAPESICILDLEVFLLGVVYTSHLQLKEKCNSHHSSYQPLCLPLPVCKQLCTERQKSWWDAVCTLIHRKAVPGNVAKLRLLVQHEINTLRAQEKHGLQPALLVHWAECLQKTGSGLNSFYDQREYIGRSVHYWKKVLPLLKIIKKKNSIPEPIDPLFKHFHSVDIQASEIVEYEEDAHITFAILDAVNGNIEDAVTAFESIKSVVSYWNLALIFHRKAEDIENDALSPEEQEECKNYLRKTRDYLIKIIDDSDSNLSVVKKLPVPLESVKEMLNSVMQELEDYSEGGPLYKNGSLRNADSEIKHSTPSPTKYSLSPSKSYKYSPKTPPRWAEDQNSLLKMICQQVEAIKKEMQELKLNSSNSASPHRWPTENYGPDSVPDGYQGSQTFHGAPLTVATTGPSVYYSQSPAYNSQYLLRPAANVTPTKGPVYGMNRLPPQQHIYAYPQQMHTPPVQSSSACMFSQEMYGPPALRFESPATGILSPRGDDYFNYNVQQTSTNPPLPEPGYFTKPPIAAHASRSAESKTIEFGKTNFVQPMPGEGLRPSLPTQAHTTQPTPFKFNSNFKSNDGDFTFSSPQVVTQPPPAAYSNSESLLGLLTSDKPLQGDGYSGAKPIPGGQTIGPRNTFNFGSKNVSGISFTENMGSSQQKNSGFRRSDDMFTFHGPGKSVFGTPTLETANKNHETDGGSAHGDDDDDGPHFEPVVPLPDKIEVKTGEEDEEEFFCNRAKLFRFDVESKEWKERGIGNVKILRHKTSGKIRLLMRREQVLKICANHYISPDMKLTPNAGSDRSFVWHALDYADELPKPEQLAIRFKTPEEAALFKCKFEEAQSILKAPGTNVAMASNQAVRIVKEPTSHDNKDICKSDAGNLNFEFQVAKKEGSWWHCNSCSLKNASTAKKCVSCQNLNPSNKELVGPPLAETVFTPKTSPENVQDRFALVTPKKEGHWDCSICLVRNEPTVSRCIACQNTKSANKSGSSFVHQASFKFGQGDLPKPINSDFRSVFSTKEGQWDCSACLVQNEGSSTKCAACQNPRKQSLPATSIPTPASFKFGTSETSKTLKSGFEDMFAKKEGQWDCSSCLVRNEANATRCVACQNPDKPSPSTSVPAPASFKFGTSETSKAPKSGFEGMFTKKEGQWDCSVCLVRNEASATKCIACQNPGKQNQTTSAVSTPASSETSKAPKSGFEGMFTKKEGQWDCSVCLVRNEASATKCIACQNPGKQNQTTSAVSTPASSETSKAPKSGFEGMFTKKEGQWDCSVCLVRNEASATKCIACQCPSKQNQTTAISTPASSEISKAPKSGFEGMFIRKGQWDCSVCCVQNESSSLKCVACDASKPTHKPIAEAPSAFTLGSEMKLHDSSGSQVGTGFKSNFSEKASKFGNTEQGFKFGHVDQENSPSFMFQGSSNTEFKSTKEGFSIPVSADGFKFGISEPGNQEKKSEKPLENGTGFQAQDISGQKNGRGVIFGQTSSTFTFADLAKSTSGEGFQFGKKDPNFKGFSGAGEKLFSSQYGKMANKANTSGDFEKDDDAYKTEDSDDIHFEPVVQMPEKVELVTGEEDEKVLYSQRVKLFRFDAEVSQWKERGLGNLKILKNEVNGKLRMLMRREQVLKVCANHWITTTMNLKPLSGSDRAWMWLASDFSDGDAKLEQLAAKFKTPELAEEFKQKFEECQRLLLDIPLQTPHKLVDTGRAAKLIQRAEEMKSGLKDFKTFLTNDQTKVTEEENKGSGTGAAGASDTTIKPNPENTGPTLEWDNYDLREDALDDSVSSSSVHASPLASSPVRKNLFRFGESTTGFNFSFKSALSPSKSPAKLNQSGTSVGTDEESDVTQEEERDGQYFEPVVPLPDLVEVSSGEENEQVVFSHRAKLYRYDKDVGQWKERGIGDIKILQNYDNKQVRIVMRRDQVLKLCANHRITPDMTLQNMKGTERVWLWTACDFADGERKVEHLAVRFKLQDVADSFKKIFDEAKTAQEKDSLITPHVSRSSTPRESPCGKIAVAVLEETTRERTDVIQGDDVADATSEVEVSSTSETTPKAVVSPPKFVFGSESVKSIFSSEKSKPFAFGNSSATGSLFGFSFNAPLKSNNSETSSVAQSGSESKVEPKKCELSKNSDIEQSSDSKVKNLFASFPTEESSINYTFKTPEKAKEKKKPEDSPSDDDVLIVYELTPTAEQKALATKLKLPPTFFCYKNRPDYVSEEEEDDEDFETAVKKLNGKLYLDGSEKCRPLEENTADNEKECIIVWEKKPTVEEKAKADTLKLPPTFFCGVCSDTDEDNGNGEDFQSELQKVQEAQKSQTEEITSTTDSVYTGGTEVMVPSFCKSEEPDSITKSISSPSVSSETMDKPVDLSTRKEIDTDSTSQGESKIVSFGFGSSTGLSFADLASSNSGDFAFGSKDKNFQWANTGAAVFGTQSVGTQSAGKVGEDEDGSDEEVVHNEDIHFEPIVSLPEVEVKSGEEDEEILFKERAKLYRWDRDVSQWKERGVGDIKILWHTMKNYYRILMRRDQVFKVCANHVITKTMELKPLNVSNNALVWTASDYADGEAKVEQLAVRFKTKEVADCFKKTFEECQQNLMKLQKGHVSLAAELSKETNPVVFFDVCADGEPLGRITMELFSNIVPRTAENFRALCTGEKGFGFKNSIFHRVIPDFVCQGGDITKHDGTGGQSIYGDKFEDENFDVKHTGPGLLSMANQGQNTNNSQFVITLKKAEHLDFKHVVFGFVKDGMDTVKKIESFGSPKGSVCRRITITECGQI

SEQ ID NO: 27 - DNA, 10697 bp, Homo sapiens
  ref <308>: DATABASE ACCESSION NUMBER: NM-006267
  ref <309>: DATABASE ENTRY DATE: 2005-04-22
  ref <313>: RELEVANT RESIDUES IN SEQ ID NO: (1)..(10697)
cacagtggtcctccgccggctacggcgctgcgtcactggtttgcaggcgctttcctcttggaagtggcgactgctgcgggcctgagcgctggtctcacgcgcctcgggagccaggttggcggcgcgatgaggcgcagcaaggctgacgtggagcggtacatcgcctcggtgcagggctccaccccgtcgcctcgacagaagtcaatgaaaggattctattttgcaaagctgtattatgaagctaaagaatatgatcttgctaaaaaatacatatgtacttacattaatgtgcaagagagggatcccaaagctcacagatttctgggtcttctttatgaattggaagaaaacacagacaaagccgttgaatgttacaggcgttcagtggaattaaacccaacacaaaaagatcttgtgttgaagattgcagaattgctttgtaaaaatgatgttactgatggaagagcaaaatactggcttgaaagagcagccaaacttttcccaggaagtcctgcaatttataaactaaaggaacagcttctagattgtgaaggtgaagatggatggaataaactttttgacttgattcagtcagaactttatgtaagacctgatgacgtccatgtgaacatccggctagtggaggtgtatcgctcaactaaaagattgaaggatgctgtggcccactgccatgaggcagagaggaacatagctttgcgttcaagtttagaatggaattcgtgtgttgtacagacccttaaggaatatctggagtctttacagtgtttggagtctgataaaagtgactggcgagcaaccaatacagacttactgctggcctatgctaatcttatgcttcttacgctttccactagagatgtgcaggaaagtagagaattactgcaaagttttgatagtgctcttcagtctgtgaaatctttgggtggaaatgatgaactgtcagctactttcttagaaatgaaaggacatttctacatgcatgctggttctctgcttttgaagatgggtcagcatagtagtaatgttcaatggcgagctctttctgagctggctgcattgtgctatctcatagcatttcaggttccaagaccaaagattaaattaataaaaggtgaagctggacaaaatctgctggaaatgatggcctgtgaccgactgagccaatcagggcacatgttgctaaacttaagtcgtggcaagcaagattttttaaaagagattgttgaaacttttgccaacaaaagcgggcagtctgcattatatgatgctctgttttctagtcagtcacctaaggatacatcttttcttggtagcgatgatattggaaacattgatgtacgagaaccagagcttgaagatttgactagatacgatgttggtgctattcgagcacataatggtagtcttcagcaccttacttggcttggcttacagtggaattcattgcctgctttacctggaatccgaaaatggctaaaacagcttttccatcatttgccccatgaaacctcaaggcttgaaacaaatgcacctgaatcaatatgtattttagatcttgaagtatttctccttggagtagtatataccagccacttacaattaaaggagaaatgtaattctcaccacagctcctatcagccgttatgcctgccccttcctgtgtgtaaacagctttgtacagaaagacaaaaatcttggtgggatgcggtttgtactctgattcacagaaaagcagtacctggaaacgtagcaaaattgagacttctagttcagcatgaaataaacactctaagagcccaggaaaaacatggccttcaacctgctctgcttgtacattgggcagaatgccttcagaaaacgggcagcggtcttaattctttttatgatcaacgagaatacatagggagaagtgttcattattggaagaaagttttgccattgttgaagataataaaaaagaagaacagtattcctgaacctattgatcctctgtttaaacattttcatagtgtagacattcaggcatcagaaattgttgaatatgaagaagacgcacacataacttttgctatattggatgcagtaaatggaaatatagaagatgctgtgactgcttttgaatctataaaaagtgttgtttcttattggaatcttgcactgatttttcacaggaaggcagaagacattgaaaatgatgccctttctcctgaagaacaagaagaatgcaaaaattatctgagaaagaccagggactacctaataaagattatagatgacagtgattcaaatctttcagtggtcaagaaattgcctgtgcccctggagtctgtaaaagagatgcttaattcagtcatgcaggaactcgaagactatagtgaaggaggtcctctctataaaaatggttctttgcgaaatgcagattcagaaataaaacattctacaccgtctcctaccaaatattcactatcaccaagtaaaagttacaagtattctcccaaaacaccacctcgatgggcagaagatcagaattctttactgaaaatgatttgccaacaagtagaggccattaagaaagaaatgcaggagttgaaactaaatagcagtaactcagcatcccctcatcgttggcccacagagaattatggaccagactcggtgcctgatggatatcaggggtcacagacatttcatggggctccactaacagttgcaactactggcccttcagtatattatagtcagtcaccagcatataattcccagtatcttctcagaccagcagctaatgttactcccacaaagggcccagtctatggcatgaataggcttccaccccaacagcatatttatgcctatccgcaacagatgcacacaccgccagtgcaaagctcatctgcttgtatgttctctcaggagatgtatggtcctcctgcattgcgttttgagtctcctgcaacgggaattctatcgcccaggggtgatgattactttaattacaatgttcaacagacaagcacaaatccacctttgccagaaccaggatatttcacaaaacctccgattgcagctcatgcttcaagatctgcagaatctaagactatagaatttgggaaaactaattttgttcagcccatgccgggtgaaggattaaggccatctttgccaacacaagcacacacaacacagccaactccttttaaatttaactcaaatttcaaatcaaatgatggtgacttcacgttttcctcaccacaggttgtgacacagccccctcctgcagcttacagtaacagtgaaagccttttaggtctcctgacttcagataaacccttgcaaggagatggctatagtggagccaaaccaattcctggtggtcaaaccattgggcctcgaaatacattcaattttggaagcaaaaatgtgtctggaatttcatttacagaaaacatggggtcgagtcagcaaaagaattctggttttcggcgaagtgatgatatgtttactttccatggtccagggaaatcagtatttggaacacccactttagagacagcaaacaagaatcatgagacagatggaggaagtgcccatggggatgatgatgatgacggtcctcactttgagcctgtagtacctcttcctgataagattgaagtaaaaactggtgaggaagatgaagaagaattcttttgcaaccgcgcgaaattgtttcgtttcgatgtagaatccaaagaatggaaagaacgtgggattggcaatgtaaaaatactgaggcataaaacatctggtaaaattcgccttctaatgagacgagagcaagtattgaaaatctgtgcaaatcattacatcagtccagatatgaaattgacaccaaatgctggatcagacagatcttttgtatggcatgcccttgattatgcagatgagttgccaaaaccagaacaacttgctattaggttcaaaactcctgaggaagcagcactttttaaatgcaagtttgaagaagcccagagcattttaaaagccccaggaacaaatgtagccatggcgtcaaatcaggctgtcagaattgtaaaagaacccacaagtcatgataacaaggatatttgcaaatctgatgctggaaacctgaattttgaatttcaggttgcaaagaaagaagggtcttggtggcattgtaacagctgctcattaaagaatgcttcaactgctaagaaatgtgtatcatgccaaaatctaaacccaagcaataaagagctcgttggcccaccattagctgaaactgtttttactcctaaaaccagcccagagaatgttcaagatcgatttgcattggtgactccaaagaaagaaggtcactgggattgtagtatttgtttagtaagaaatgaacctactgtatctaggtgcattgcgtgtcagaatacaaaatctgctaacaaaagtggatcttcatttgttcatcaagcttcatttaaatttggccagggagatcttcctaaacctattaacagtgatttcagatctgttttttctacaaaggaaggacagtgggattgcagtgcatgtttggtacaaaatgaggggagctctacaaaatgtgctgcttgtcagaatccgagaaaacagagtctacctgctacttctattccaacacctgcctcttttaagtttggtacttcagagacaagtaaaactctaaaaagtggatttgaagacatgtttgctaagaaggaaggacagtgggattgcagttcatgcttagtgcgaaatgaagcaaatgctacaagatgtgttgcttgtcagaatccggataaaccaagtccatctacttctgttccagctcctgcctcttttaagtttggtacttcagagacaagcaaggctccaaagagcggatttgagggaatgttcactaagaaggagggacagtgggattgcagtgtgtgcttagtaagaaatgaagccagtgctaccaaatgtattgcttgtcagaatccaggtaaacaaaatcaaactacttctgcagtttcaacacctgcctcttcagagacaagcaaggctccaaagagcggatttgagggaatgttcactaagaaggagggacagtgggattgcagtgtgtgcttagtaagaaatgaagccagtgctaccaaatgtattgcttgtcagaatccaggtaaacaaaatcaaactacttctgcagtttcaacacctgcctcttcagagacaagcaaggctccaaagagcggatttgagggaatgttcactaagaaggaaggacagtgggattgcagtgtgtgcttagtaagaaatgaagccagtgctaccaaatgtattgcttgtcagtgtccaagtaaacaaaatcaaacaactgcaatttcaacacctgcctcttcggagataagcaaggctccaaagagtggatttgaaggaatgttcatcaggaaaggacagtgggattgtagtgtttgctgtgtacaaaatgagagttcttccttaaaatgtgtggcttgtgatgcctctaaaccaactcataaacctattgcagaagctccttcagctttcacactgggctcagaaatgaagttgcatgactcttctggaagtcaggtgggaacaggatttaaaagtaatttctcagaaaaagcttctaagtttggcaatacagagcaaggattcaaatttgggcatgtggatcaagaaaattcaccttcatttatgtttcagggttcttctaatacagaatttaagtcaaccaaagaaggattttccatccctgtgtctgctgatggatttaaatttggcatttcggaaccaggaaatcaagaaaagaaaagtgaaaagcctcttgaaaatggtactggcttccaggctcaggatattagtggccagaagaatggccgtggtgtgatttttggccaaacaagtagcacttttacatttgcagatcttgcaaaatcaacttcaggagaaggatttcagtttggcaaaaaagaccccaatttcaagggattttcaggtgctggagaaaaattattctcatcacaatacggtaaaatggccaataaagcaaacacttccggtgactttgagaaagatgatgatgcctataagactgaggacagcgatgacatccattttgaaccagtagttcaaatgcccgaaaaagtagaacttgtaacaggagaagaagatgaaaaagttctgtattcacagcgggtaaaactatttagatttgatgctgaggtaagtcagtggaaagaaaggggcttggggaacttaaaaattctcaaaaacgaggtcaatggcaaactaagaatgctgatgcgaagagaacaagtactaaaagtgtgtgctaatcattggataacgactacgatgaacctgaagcctctctctggatcagatagagcatggatgtggttagccagtgatttctctgatggtgatgccaaactagagcagttggcagcaaaatttaaaacaccagagctggctgaagaattcaagcagaaatttgaggaatgccagcggcttctgttagacataccacttcaaactccccataaacttgtagatactggcagagctgccaagttaatacagagagctgaagaaatgaagagtggactgaaagatttcaaaacatttttgacaaatgatcaaacaaaagtcactgaggaagaaaataagggttcaggtacaggtgcggccggtgcctcagacacaacaataaaacccaatcctgaaaacactgggcccacattagaatgggataactatgatttaagggaagatgctttggatgatagtgtcagtagtagctcagtacatgcttctccattggcaagtagccctgtgagaaaaaatcttttccgttttggtgagtcaacaacaggatttaacttcagttttaaatctgctttgagtccatctaagtctcctgccaagttgaatcagagtgggacttcagttggcactgatgaagaatctgatgttactcaagaagaagagagagatggacagtactttgaacctgttgttcctttacctgatctagttgaagtatccagtggtgaggaaaatgaacaagttgtttttagtcacagggcaaaactctacagatatgataaagatgttggtcaatggaaagaaaggggcattggtgatataaagattttacagaattatgataataagcaagttcgtatagtgatgagaagggaccaagtattaaaactttgtgccaatcacagaataactccagacatgactttgcaaaatatgaaagggacagaaagagtatggttgtggactgcatgtgattttgcagatggagaaagaaaagtagagcatttagctgttcgttttaaactacaggatgttgcagactcgtttaagaaaatttttgatgaagcaaaaacagcccaggaaaaagattctttgataacacctcatgtttctcggtcaagcactcccagagagtcaccatgtggcaaaattgctgtagctgtattagaagaaaccacaagagagaggacagatgttattcagggtgatgatgtagcagatgcaacttcagaagttgaagtgtctagcacatctgaaacaacaccaaaagcagtggtttctcctccaaagtttgtatttggttcagagtctgttaaaagcatttttagtagtgaaaaatcaaaaccatttgcattcggcaacagttcagccactgggtctttgtttggatttagttttaatgcacctttgaaaagtaacaatagtgaaactagttcagtagcccagagtggatctgaaagcaaagtggaacctaaaaaatgtgaactgtcaaagaactctgatatcgaacagtcttcagatagcaaagtcaaaaatctctttgcttcctttccaacggaagaatcttcaatcaactacacatttaaaacaccagaaaaggcaaaagagaagaaaaaacctgaagattctccctcagatgatgatgttctcattgtatatgaactaactccaaccgctgagcagaaagcccttgcaaccaaacttaaacttcctccaactttcttctgctacaagaatagaccagattatgttagtgaagaagaggaggatgatgaagatttcgaaacagctgtcaagaaacttaatggaaaactatatttggatggctcagaaaaatgtagacccttggaagaaaatacagcagataatgagaaagaatgtattattgtttgggaaaagaaaccaacagttgaagagaaggcaaaagcagatacgttaaaacttccacctacatttttttgtggagtctgtagtgatactgatgaagacaatggaaatggggaagactttcaatcagagcttcaaaaagttcaggaagctcaaaaatctcagacagaagaaataactagcacaactgacagtgtatatacaggtgggactgaagtgatggtaccttctttctgtaaatctgaagaacctgattctattaccaaatccattagttcaccatctgtttcctctgaaactatggacaaacctgtagatttgtcaactagaaaggaaattgatacagattctacaagccaaggggaaagcaagatagtttcatttggatttggaagtagcacagggctctcatttgcagacttggcttccagtaattctggagattttgcttttggttctaaagataaaaatttccaatgggcaaatactggagcagctgtgtttggaacacagtcagtcggaacccagtcagccggtaaagttggtgaagatgaagatggtagtgatgaagaagtagttcataatgaagatatccattttgaaccaatagtgtcactaccagaggtagaagtaaaatctggagaagaagatgaagaaattttgtttaaagagagagccaaactttatagatgggatcgggatgtcagtcagtggaaggagcgcggtgttggagatataaagattctttggcatacaatgaagaattattaccggatcctaatgagaagagaccaggtttttaaagtgtgtgcaaaccacgttattactaaaacaatggaattaaagcccttaaatgtttcaaataatgctttagtttggactgcctcagattatgctgatggagaagcaaaagtagaacagcttgcagtgagatttaaaactaaagaagtagctgattgtttcaagaaaacatttgaagaatgtcagcagaatttaatgaaactccagaaaggacatgtatcactggcagcagaattatcaaaggagaccaatcctgtggtgttttttgatgtttgtgcggacggtgaacctctagggcggataactatggaattattttcaaacattgttcctcggactgctgagaacttcagagcactatgcactggagagaaaggctttggtttcaagaattccatttttcacagagtaattccagattttgtttgccaaggaggagatatcaccaaacatgatggaacaggcggacagtccatttatggagacaaatttgaagatgaaaattttgatgtgaaacatactggtcctggtttactatccatggccaatcaaggccagaataccaataattctcaatttgttataacactgaagaaagcagaacatttggactttaagcatgtagtatttgggtttgttaaggatggcatggatactgtgaaaaagattgaatcatttggttctcccaaagggtctgtttgtcgaagaataactatcacagaatgtggacagatataaaatcattgttgttcatagaaaatttcatctgtataagcagttggattgaagcttagctattacaatttgatagttatgttcagcttttgaaaatggacgtttccgatttacaaatgtaaaattgcagcttatagctgttgtcactttttaatgtgttataattgaccttgcatggtgtgaaataaaagtttaaacactggtgatttcaggtgtacttgtgtttatgtactcctgacgtattaaaatggaataatactaatcttgttaaaagcaatagacctcaaactattgaaggaatatgatatatgcaatttaattttaattccttttaagatatttggacttcctgcatggatatacttaccatttgaataaagggaccacaacttggataatttaattttaggtttgaaatatatttggtaatcttaactattggtgtactcatttatgcatagagactcgtttatgaatgggtagagccacagaacgtatagagttaaccaaagtgctcttctctagaatctttacacctcctgtgtggttacaagttaactttgtaagtagcgtaccttccttccttaaaatatctagcttcctgtgccctttcatagatattcgattaatttttacattttaaacaagttgactatttcctttaggggttttgtttcaaacttttctgtcatctgtctctactacctcagaaactgcagcttggttctgatgatagaaattgaatttttccttgtagttattgtgataaagtatgaatatttttagaaagtctataccatgttctttcgttaaagatttgctttatacaagattgttgcagtacctttttctggtaaattttgtagcagaaataaaatgacaattcctaagagccaaaa

SEQ ID NO: 28 - protein, 2090 aa, Homo sapiens [sequence omitted]

SEQ ID NO: 29 - DNA, 6614 bp, Homo sapiens
  ref <308>: DATABASE ACCESSION NUMBER: NM_005085
  ref <309>: DATABASE ENTRY DATE: 2005-04-22
  ref <313>: RELEVANT RESIDUES IN SEQ ID NO: (1)..(6614)
ctgcgcgccgctggcgctgaggggaggaagtttgctgtcgagcggcctgggttccgtgggcaaggccgtgggaggcagcgttggctgcttcgacacactgagggcggcgcgatgggagacgagatggatgccatgattcccgagcgggagatgaaggattttcagtttagagcgctaaagaaggtgagaatctttgactcccctgaggaattgcccaaggaacgctcgagtctgcttgctgtgtccaacaaatatggtctggtcttcgctggtggagccagtggcttgcagatttttcctactaaaaatcttcttattcaaaataaacccggagatgatcccaacaaaatagttgataaagtccaaggcttgctagttcctatgaaattcccaatccatcacctggccttgagctgtgataacctcacactctctgcgtgcatgatgtccagtgaatatggttccattattgctttttttgatgttcgcacattctcaaatgaggctaaacagcaaaaacgcccatttgcctatcataagcttttgaaagatgcaggaggcatggtgattgatatgaagtggaaccccactgtcccctccatggtggcagtttgtctggctgatggtagtattgctgtcctgcaagtcacggaaacagtgaaagtatgtgcaactcttccttccacggtagcagtaacctctgtgtgctggagccccaaaggaaagcagctggcagtgggaaaacagaatggaactgtggtccagtatcttcctactttgcaggaaaaaaaagtcattccttgtcctccgttttatgagtcagatcatcctgtcagagttctggatgtgctgtggattggtacctacgtcttcgccatagtgtatgctgctgcagatgggaccctggaaacgtctccagatgtggtgatggctctactaccgaaaaaagaagaaaagcacccagagatatttgtgaactttatggagccctgttatggcagctgcacggagagacagcatcattactacctcagttacattgaggaatgggatttagtgctggcagcatctgcggcttcaacagaagttagtatccttgctcgacaaagtgatcagattaattgggaatcttggctactggaggattctagtcgagctgaattgcctgtgacagacaagagtgatgactccttgcccatgggagttgtcgtagactatacaaaccaagtggaaatcaccatcagtgatgaaaagactcttcctcctgctccagttctcatgttactttcaacagatggtgtgctttgtccattttatatgattaatcaaaatcctggggttaagtctctcatcaaaacaccagagcgactttcattagaaggagagcgacagcccaagtcaccaggaagtactcccactaccccaacctcctctcaagccccacagaaactggatgcttctgcagctgcagcccctgcctctctgccaccttcatcacctgctgctcccattgccactttttctttgcttcctgctggtggagcccccactgtgttctcctttggttcttcatctttgaagtcatctgctacggtcactggggagcccccttcatattccagtggctccgacagctccaaagcagccccaggccctggcccatcaaccttctcttttgttcccccttctaaagcctccctagcccccacccctgcagcgtctcctgtggctccatcagctgcttcattctcctttggatcatctggttttaagcctaccctggaaagcacaccagtgccaagtgtgtctgctccaaatatagcaatgaagccctccttcccaccctcaacctctgctgtcaaagtcaaccttagtgaaaagtttactgctgcagctacctctactcctgttagtagctcccagagcgcacccccgatgtcgccattctcttctgcctccaagccagctgcttctggaccactcagccaccccacacctctctcagcaccacctagttccgtgccattgaagtcctcagtcttgccctcaccatcaggacgatctgctcagggcagttcaagcccagtgccctcaatggtacagaaatcacccaggataacccctccagcggcaaagccaggctctccccaggcaaagtcacttcagcctgctgttgcagaaaagcagggacatcagtggaaagattcagatcctgtaatggctggaattggggaggagattgcacactttcagaaggagttggaagagttaaaagcccgaacttccaaagcctgtttccaagtgggcacttctgaggagatgaagatgctgcgaacagaatcagatgacttgcatacctttcttttggagattaaagagaccacagagtcgcttcatggagatataagtagcctgaaaacaactttacttgagggctttgctggtgttgaggaagccagagaacaaaatgaaagaaatcgtgactctggttatctgcatttgctttataaaagaccactggatcccaagagtgaagctcagcttcaggaaattcggcgccttcatcagtatgtgaaatttgctgtccaagatgtgaatgatgttctagacttggagtgggatcagcatctggaacaaaagaaaaaacaaaggcacctgcttgtgccagagcgagagacactgtttaacaccctagccaacaatcgggaaatcatcaaccaacagaggaagaggctgaatcacctggtggatagtcttcagcagctccgcctttacaaacagacttccctgtggagcctgtcctcggctgttccttcccagagcagcattcacagttttgacagtgacctggaaagcctgtgcaatgctttgttgaaaaccaccatagaatctcacaccaaatccttgcccaaagtaccagccaaactgtcccccatgaaacaggcacaactgagaaacttcttggccaagaggaagaccccaccagtgagatccactgctccagccagcctgtctcgatcagcctttctgtctcagagatattatgaagacttggatgaagtcagctcaacgtcatctgtctcccagtctctggagagtgaagatgcacggacgtcctgtaaagatgacgaggcagtggttcaggcccctcggcacgcccccgtggttcgcactccttccatccagcccagtctcttgccccatgcagcaccttttgctaaatctcacctggttcatggttcttcacctggtgtgatgggaacttcagtggctacatctgctagcaaaattattcctcaaggggccgatagcacaatgcttgccacgaaaaccgtgaaacatggtgcacctagtccttcccaccccatctcagccccgcaggcagctgccgcagcagcactcaggcggcagatggccagtcaggcaccagctgtaaacactttgactgaatcaacgttgaagaatgtccctcaagtggtaaatgtgcaggaattgaagaataaccctgcaaccccttctacagccatgggttcttcagtgccctactccacagccaaaacacctcacccagtgttgaccccagtggctgctaaccaagccaagcaggggtctctaataaattcccttaagccatctgggcctacaccagcatccggtcagttatcatctggtgacaaagcttcagggacagccaagatagaaacagctgtgacttcaaccccatctgcttctgggcagttcagcaagcctttctcattttctccatcagggactggctttaattttgggataatcacaccaacaccgtcttctaatttcactgctgcacaaggggcaacaccctccactaaagagtcaagccagccggacgcattctcatctggtgggggaagcaaaccttcttatgaggccattcctgaaagctcacctccctcaggaatcacatccgcatcaaacaccaccccaggagaacctgccgcatctagcagcagacctgtggcaccttctggaactgctctttccaccacctctagtaagctggaaaccccaccgtccaagctgggagagcttctgtttccaagttctttggctggagagactctgggaagtttttcaggactgcgggttggccaagcagatgattctacaaaaccaaccaataaggcttcatccacaagcctaactagtacccagccaaccaagacgtcaggcgtgccctcagggtttaattttactgcccccccggtgttagggaagcacacggagccccctgtgacatcctctgcaaccaccacctcagtagcaccaccagcagccaccagcacttcctcaactgccgtttttggcagtctgccagtcaccagtgcaggatcctctggggtcatcagttttggtgggacatctctaagtgctggcaagactagtttttcatttggaagccaacagaccaatagcacagtgcccccatctgccccaccaccaactacagctgccactccccttccaacatcattccccacattgtcatttggtagcctcctgagttcagcaactaccccctccctgcctatgtccgctggcagaagcacagaagaggccacttcatcagctttgcctgagaagccaggtgacagtgaggtctcagcatcagcagcctcacttctagaggagcaacagtcagcccagcttccccaggctcctccgcaaacttctgactctgttaaaaaagaacctgttcttgcccagcctgcagtcagcaactctggcactgcagcatctagtactagtcttgtagcactttctgcagaggctaccccagccaccacgggggtccctgatgccaggacggaggcagtaccacctgcttcctccttttctgtgcctgggcagactgctgtcacagcagctgctatctcaagtgcaggccctgtggccgtcgaaacatcaagtacccccatagcctccagcaccacgtccattgttgctcccggcccatctgcagaggcagcagcatttggtaccgtcacttctggctcatccgtctttgctcagcctcctgctgccagttctagctcagctttcaaccagctcaccaacaacacagccactgccccctctgccacgcccgtgtttgggcaagtggcagccagcaccgcaccaagtctgtttgggcagcagactggtagcacagccagcacagcagctgccacaccacaggtcagcagctcagggtttagcagcccagcttttggtaccacagccccaggggtctttggacagacaaccttcgggcaggcctcagtctttgggcagtcggcgagcagtgctgcaagtgtcttttccttcagtcagcctgggttcagttccgtgcctgccttcggtcagcctgcttcctccactcccacatccaccagtggaagtgtctttggtgccgcctcaagtaccagtagctccagttccttctcatttggacagtcttctcccaacacaggaggggggctgtttggccaaagcaacgctcctgcttttgggcagagtcctggctttggacagggaggctctgtctttggtggtacctcagctgccaccacaacagcagcaacctctgggttcagcttttgccaagcttcaggttttgggtctagtaatactggttctgtgtttggtcaagcagccagtactggtggaatagtctttggccagcaatcatcctcttccagtggtagcgtgtttgggtctggaaacactggaagagggggaggtttcttcagtggccttggaggaaaacccagtcaggatgcagccaacaaaaacccattcagctcggccagtgggggctttggatccacagctacctcaaatacctctaacctatttggaaacagtggggccaagacatttggtggatttgccagctcgtcgtttggagagcagaaacccactggcactttcagctctggaggaggaagtgtggcatcccaaggctttgggttttcctctccaaacaaaacaggtggcttcggtgctgctccagtgtttggcagccctcctacttttgggggatcccctgggtttggaggggtgccagcattcggttcagccccagcctttacaagccctctgggctcgacgggaggcaaagtgttcggagagggcactgcagctgccagcgcaggaggattcgggtttgggagcagcagcaacaccacatccttcggcacgctcgcgagtcagaatgcccccactttcggatcactgtcccaacagacttctggttttgggacccagagtagcggattctctggttttggatcaggcacaggagggttcagctttgggtcaaataactcgtctgtccagggttttggtggctggcgaagctgagggcgtgtcagcaggcctttcgatccctgggaccaaccgcatcctcagcttcttccccgagaaatgctggagcaggctgttcagaccgacgttgccatcaaaacacatacacccagaaagaaacaacagaaaccaaaactcacaaggcgcatgattacttgttttatatttcatgttgggttttccctcccactattaaacagtctgtttccgtacaaaaaaaaaaaaaa

SEQ ID NO: 30 - protein, 1726 aa, Homo sapiens
  ref <308>: DATABASE ACCESSION NUMBER: NM_624358
  ref <309>: DATABASE ENTRY DATE: 2005-05-16
  ref <313>: RELEVANT RESIDUES IN SEQ ID NO: (1)..(1726)
MFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVALTDPNASAAQQAVLQQHINSLTYSPFGDSPLFRNPMSDPKKKEERLKPTNPAAQKALTTPTHYKLTPRPATRVRPKALQTTGTAKSHLFDGLDDDEPSLANGAFMPKKSIKKLVLKNLNNSNLFSPVNRDSENLASPSEYPENGERFSFLSKPVDENHQQDGDEDSLVSHFYTNPIAKPIPQTPESAGNKHSNSNSVDDTIVALNMRAALRNGLEGSSEETSFHDESLQDDREEIENNSYHMHPAGIILTKVGYYTIPSMDDLAKITNEKGECIVSDFTIGRKGYGSIYFEGDVNLTNLNLDDIVHIRRKEVVVYLDDNQKPPVGEGLNRKAEVTLDGVWPTDKTSRCLIKSPDRLADINYEGRLEAVSRKQGAQFKEYRPETGSWVFKVSHFSKYGLQDSDEEEEEHPSKTSTKKLKTAPLPPASQTTPLQMALNGKPAPPPQSQSPEVEQLGRVVELDSDMVDITQEPVLDTMLEESMPEDQEPVSASTHIASSLGINPHVLQIMKASLLTDEEDVDMALDQRFSRLPSKADTSQEICSPRLPISASHSSKTRSLVGGLLQSKFTSGAFLSPSVSVQECRTPRAASLMNIPSTSSWSVPPPLTSVFTMPSPAPEVPLKTVGTRRQLGLVPREKSVTYGKGKLLMDMALFMGRSFRVGWGPNWTLANSGEQLNGSHELENHQIADSMEFGFLPNPVAVKPLTESPFKVHLEKLSLRQRKPDEDMKLYQTPLELKLKHSTVHVDELCPLIVPNLGVAVIHDYADWVKEASGDLPEAQIVKHWSLTWTLCEALWGHLKELDSQLNEPREYIQILERRRAFSRWLSCTATPQIEEEVSLTQKNSPVEAVFSYLTGKRISEACSLAQQSGDHRLALLLSQFVGSQSVRELLTMQLVDWHQLQADSFIQDERLRIFALLAGKPVWQLSEKKQINVCSQLDWKRSLAIHLWYLLPPTASISRALSMYEEAFQNTSDSDRYACSPLPSYLEGSGCVIAEEQNSQTPLRDVCFHLLKLYSDSIREKAVRELLTRHCQLLETPESWAKETFLTQKLRVPAKWIHEAKAVRAHMESDKHLEALCLFKAEHWNRCHKLIIRHLASDAIINENYDYLKGFLEDLAPPERSSLIQDWETSGLVYLDYIRVIEMLRHIQQVDCSGNDLEQLHIKVTSLCSRIEQIQCYSAKDRLAQSDMAKRVANLLRVVLSLHHPPDRTSDSTPDPQRVPLRLLAPHIGRLPMPEDYAMDELRSLTQSYLRELAVGSL

SEQ ID NO: 31 - DNA, 6628 bp, Homo sapiens
  ref <308>: DATABASE ACCESSION NUMBER: NM_139132
  ref <309>: DATABASE ENTRY DATE: 2005-05-16
  ref <313>: RELEVANT RESIDUES IN SEQ ID NO: (1)..(6628)
gcccaatgaataacagggtcgacccagagtggcggccgcgtccaccaattcgcggacggcgagggcggagcgtccgcaacgagcctctgacgccgggcggcgtgtgacgcagtcgggccctctgcgcgctgcgcccgaagcggcggtcggtggcaggggtggtagcggcggcggcgacggtttcgtgggggccgcgcgctgctctgtgagcggcgggtggcagcaggggactcctgacacttccccttccccaccgaaccgcgctttctgaaacaaagactcattttgaagatgtttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagctttgacagatccaaatgcttctgctgcccagcaggctgttctccagcagcacatcaatagtctaacatactcaccttttggagactctcctctcttccggaatccgatgtcagaccctaagaagaaggaagagagattgaaaccaacaaatccagcagcccagaaggctcttactacacctactcattataaactgacaccccgccctgccactagagtccggccaaaggctttacaaacaacaggcacagccaagtcacatctctttgatgggctggatgacgatgaaccatccctagccaatggagcattcatgcccaagaagagcattaagaagttggttttgaagaaccttaataatagcaatctcttttctcctgttaatcgtgattcagaaaatctagcttcaccatctgaatatccagaaaatggagagagatttagtttcctaagcaaacctgttgatgagaatcaccagcaggatggagatgaagattcccttgtttcacatttttatactaaccctattgccaaacctattcctcaaaccccagaaagtgctggaaataaacacagcaacagcaacagtgtggatgataccattgttgcattaaacatgcgtgctgctttgcgaaatgggctggaaggaagcagtgaagaaacgtcttttcatgatgagtcacttcaggatgaccgagaagaaatagaaaataattcttaccatatgcacccagcaggtattattctcactaaggttggttactatactattccatctatggatgaccttgctaaaattaccaatgaaaaaggagagtgcattgtctctgatttcactattggtcggaaaggttatggttcaatctattttgaaggagatgtgaatttgacaaatctaaatttggatgatattgtgcatatccggaggaaagaagtagttgtctacttagatgataaccaaaaaccacctgtgggtgaagggctaaataggaaggctgaagttacattggatggagtttggccaacagataaaacatctcgttgtttaataaagagcccagatcgccttgctgatatcaactatgaaggaagattggaagcagtttcaaggaaacagggagctcaattcaaagaataccggcctgaaactggttcttgggtgtttaaggtctcccatttttctaagtatggccttcaggattctgatgaagaggaggaggagcatccgtctaaaactagtacaaagaagttgaagactgctcctttgcctcctgcaagccagactacgcccttgcagatggctcttaatggcaaacctgcacctccacctcagagccagagcccagaggtggagcagttagggagggttgtggaactggacagtgacatggtagatatcacccaggagccagttttggataccatgttagaagagagcatgcctgaggatcaggaacctgtgtctgcctcaacacatattgcatcttcactgggaattaatccacatgtcttacagatcatgaaagcatcattgcttactgatgaagaagatgtagatatggcactggatcaacgcttcagtcgcctgccttccaaagcagatacttctcaagaaatctgttctcccagactccccatttcagcatcccactcgtcgaaaactcgttcactagttggtgggttactacaatcaaaatttacaagtggagcttttctttcaccaagtgtctctgtgcaagaatgtcgtactcccagagcagcatctttaatgaatatcccatccacatcctcttggtctgtccctccacccctgacttctgtgttcacaatgcccagcccagcccctgaggttccgttgaaaacagtgggtacacgtaggcaactaggcctagtccctcgtgaaaagtctgtcacctatggcaagggaaaactcttgatggacatggccctattcatgggacgctcatttcgtgttggttggggccccaactggactcttgctaatagtggagaacagctgaatggctctcatgaactagaaaatcatcagattgccgattccatggagtttggattcctgcccaatccagtagctgttaaacccctaactgagtccccattcaaagttcacttagaaaaactcagtttgagacaaagaaagccagatgaagacatgaaattatatcagacacctctggagctcaaattaaaacacagcactgtccatgtggatgaactgtgtcctctcattgtccccaatctgggagttgctgtcattcatgactatgcagattgggttaaagaagcatcaggagacttaccagaagcacaaattgtgaagcactggagcctgacatggacactatgtgaagccctatggggccacctgaaggagcttgacagccagctaaatgaaccccgtgaatacattcaaattctggagcgaagaagagctttctcccgctggctatcctgtactgccacacctcagattgaagaggaagtctccttaacccaaaaaaacagccctgtggaggctgtattcagctacctcacaggcaaaaggatcagtgaggcctgctctctggcccagcagtcaggggatcatcgtcttgctcttcttttatctcagtttgtgggtagccagtcagtccgggagctgctcaccatgcagttggtggactggcatcagctccaagcagactccttcatccaggatgagagactgcgcatctttgctctgttggctggaaaaccggtgtggcagctctcagaaaagaagcaaataaacgtgtgctcccagttggattggaaacgctccctggctatccatctttggtatttgcttccaccaacagcctccatttctagggcgctcagcatgtatgaagaagcatttcagaatacctctgacagtgacagatatgcctgctccccacttccttcgtatctggagggttctggctgtgtgatagcggaggagcaaaactcacagacaccacttcgagatgtctgctttcaccttctaaaactctacagtgacagcatacgtgagaaggctgttcgagagctgcttacccggcactgccagctgttggagacccctgaatcttgggctaaagagactttccttacccagaagctccgtgtacctgccaaatggatccacgaggccaaagctgtgcgagcacacatggaatctgacaagcacttagaggccctttgcttatttaaggctgagcactggaaccgctgccacaagctcatcatccgacacttagcttctgatgccatcattaatgagaactatgactacctgaaggggttcttggaagacctggcacctccagagcgcagcagcctaattcaggattgggaaacatctgggcttgtttacctggactatattagagtcattgaaatgctccgccatatacagcaggtggattgctcaggtaatgacctggagcagttacacatcaaagtgacttcactgtgcagtcggatagagcagattcagtgttacagtgctaaagatcgcctggctcagtcagacatggccaaacgtgtagccaacctgctgcgcgtggtgctgagtctgcatcatcctcctgatagaacctccgactcaacaccagaccctcagcgagtccctttgcgcctcttggctccccacattggccggcttcccatgcctgaggactatgccatggacgaactgcgcagccttacccagtcctatctgcgagaactggctgttgggagcctgtgagccccaggcactttgcatcacagtcacatgcccattcacaccacacagaggttccctgccttgtttggattggcactgtttgccattctctgggttggctgtggcatctaccctccctccctgctgccagaagcagcatcctccacttgttcagggcttttcttaatactgaacgtagcataagggcttctggaacccagaagaggagacagtttaccatcctcaagatcattcagtgtttttcctttaaaaaaatggtcaataaagctcctttggcagaatcccccaaagaaccagggtattctttttccatccctagccattctggatcttgtgaccctccatgccaaccagcttccctactcctaccctggcccttttatactaggactccttaggaggagtgagacaggtgataatggatccttaacagatgaactatccacagaaggaagagggatccgtctcttaagtaattggttagttaacactgaattttggaggcaaaggagggttggcctgagttaggaaacaaaatgggatctttctgacacacttagggcagaagtgaatgcctgtcacggagggattgatcttcagggctgtttttgttcctgcctttagagttccatgaacaccatacctttgctactactatgtgcaggaacccttggtcacatgtgacatgtctgtgggaagctcccagagtttggtttggtccctggttttcagtcttgctgagactctgtctggatttgcctgcagagtttggataaaaaatggcaggttgggtaaccctccctgttcatcccatgttagctccaaagcatttcccaccctccatctaccccttccagaagcaaaaacaaaccatgactgaggcaggcatggagttgggcgttaggggcaggcagagggcctttgctacactgctgacagctatagggagccccaggtaatggcatgagatagctggtgttagggctatctcaggcaatatggccacacctgggtctttatgcatgaagataatgtaaaggtttttattaaaaaatatatatatgtataaataaatgatctagatattttcctctttttctgaagctactttcttaaaaaataaataaaatgtttatagcattcctggtaaaaaaaa

SEQ ID NO: 32 - protein, 1475 aa, Homo sapiens
  ref <308>: DATABASE ACCESSION NUMBER: S42718
  ref <309>: DATABASE ENTRY DATE: 1999-11-05
  ref <313>: RELEVANT RESIDUES IN SEQ ID NO: (1)..(1475)
MASGAGGVGGGGGGKIRTRRCHQGPIKPYQQGRQQHQGILSRVTESVKNIVPGWLQRYFNKNEDVCSCSTDTSEVPRWPENKEDHLVYADEESSNITDGRITPEPAVSNTEEPSTTSTASNYPDVLTRPSLHRSHLNFSMLESPALHCQPSTSSAFPIGSSGFSLVKEIKDSTSQHDDDNISTTSGFSSRASDKDITVSKNTSLPPLWSPEAERSHSLSQHTATSSKKPAFNLSAFGTLSPSLGNSSILKTSQLGDSPFYPGKTTYGGAAAAVRQSKLRNTPYQAPVRRQMKAKQLSAQSYGVTSSTARRILQSLEKMSSPLADAKRIPSIVSSPLNSPLDRSGIDITDFQAKREKVDSQYPPVQRLMTPKPVSIATNRSVYFKPSLTPSGEFRKTNQRIDNKCSTGYEKNMTPGQNREQRESGFSYPNFSLPAANGLSSGVGGGGGKMRRERTRFVASKPLEEEEMEVPVLPKISLPITSSSLPTFNFSSPEITTSSPSPINSSQALTNKVQMTSPSSTGSPMFKFSSPIVKSTEANVLPPSSIGFTFSVPVAKTAELSGSSSTLEPIISSSAHHVTTVNSTNCKKTPPEDCEGPFRPAEILKEGSVLDILKSPGFASPKIDSVAAQPTATSPVVYTRPAISSFSSSGIGFGESLKAGSSWQCDTCLLQNKVTDNKCIACQAAKLSPRDTAKQTGIETPNKSGKTTLSASGTGFGDKFKPVIGTWDCDTCLVQNKPEAIKCVACETPKPGTCVKRALTLTVVSESAETMTASSSSCTVTTGTLGFGDKFKRPIGSWECSVCCVSNNAEDNKCVSCMSEKPGSSVPASSSSTVPVSLPSGGSLGLEKFKKPEGSWDCELCLVQNKADSTKCLACESAKPGTKSGFKGFDTSSSSSNSAASSSFKFGVSSSSSGPSQTLTSTGNFKFGDQGGFKIGVSSDSGSINPMSEGFKFSKPIGDFKFGVSSESKPEEVKKDSKNDNFKFGLSSGLSNPVSLTPFQFGVSNLGQEEKKEELPKSSSAGFSFGTGVINSTPAPANTIVTSENKSSFNLGTIETKSASVAPFTCKTSEAKKEEMPATKGGFSFGNVEPASLPSASVFVLGRTEEKQQEPVTSTSLVFGKKADNEEPKCQPVFSFGNSEQTKDENSSKSTFSFSMTKPSEKESEQPAKATFAFGAQTSTTADQGAAKPVFSFLNNSSSSSSTPATSAGGGIFGSSTSSSNPPVATFVFGQSSNPVSSSAFGNTAESSTSQSLLFSQDSKLATTSSTGTAVTPFVFGPGASSNNTTTSGFGFGATTTSSSAGSSFVFGTGPSAPSASPAFGANQTPTFGQSQGASQPNPPGFGSISSSTALFPTGSQPAPPTFGTVSSSSQPPVFGQQPSQSAFGSGTTPNSSSAFQFGSSTTNFNFTNNSPSGVFTFGANSSTPAASAQPSGSGGFPFNQSPAAFTVGSNGKNVFSSSGTSFSGRKIKTAVRRRK

SEQ ID NO: 33 - DNA, 5687 bp, Homo sapiens
  ref <308>: DATABASE ACCESSION NUMBER: NM_005124
  ref <309>: DATABASE ENTRY DATE: 2005-05-02
  ref <313>: RELEVANT RESIDUES IN SEQ ID NO: (1)..(5687)
ttccccgctgccagcactcaccctctctccgctcctgctcgcaacctcgcggttcctggggtgcttgtgcccactgtgtggacagcgcgggcggacttttgggccggggccgggcggcgggggaggctctctaaggcctccgcctctgcctctcccgcccccttacccgccccggagcgggaagcggcggaggctccgccatggcctcgggagccggaggagtcggagggggcggtggcggcaagatccggacgcggcgttgccaccaggggccaattaagccttaccagcaggggcgacaacagcatcagggcattcttagcagggttacagaatctgttaagaatattgtgccagggtggctacaaagatacttcaacaagaatgaagatgtatgcagctgttcaacagacacaagcgaggttccacgctggccagaaaataaagaggaccatctggtatatgccgatgaggagagctctaatattactgatgggagaatcacacctgagccagcagtcagtaatacagaagaaccttcaacaactagtactgcttcaaattatccagatgtgttaacaaggccttctcttcatcggagccatctgaatttttccatgttggaatcccctgcattacactgtcagccatctacatcctcggcattcccaattggcagttcgggattttcccttgtaaaggaaattaaagattctacctctcagcatgatgatgataacatctcaactaccagtggtttttcttcaagagcttctgataaagatataactgtttcaaagaacacttcattgccacctctgtggtccccagaagctgaacgttctcactcactctcacagcacactgccaccagctcaaaaaaaccagcattcaacttgtctgcctttggaacactttccccttcacttgggaattcttcaatccttaaaaccagtcagcttggagattctcctttttatcctggaaaaacaacatacggtggggcagcagctgctgtaagacagtctaaactacgaaatacaccttatcaggcaccagttagaagacaaatgaaagctaagcaactcagtgcacaatcttacggtgtgaccagttcaacagctcggcgaatattgcagtctttagagaagatgtcaagccctttagcggatgcaaaaagaattccatccattgtttcttctcctctgaattctcctcttgataggagtgggatagatatcacagattttcaggccaaaagagaaaaggtggattctcaatatcctcctgttcagagacttatgaccccaaagccagtttccatagcaacaaatcgaagtgtttattttaaaccatctctgactccttctggtgaattcaggaagactaatcaaagaatagataacaagtgcagtactggatatgaaaaaaatatgacacccggacaaaatagagaacaacgagaaagtggcttttcatatccaaatttcagtttgcctgcagccaatggtttatcttctggagtaggtggtggaggtggcaagatgagacgagaaagaacacgctttgttgcttctaaacctctggaggaggaggaaatggaagttccagtattaccgaaaatctctctaccgatcaccagttcttcactgcctacctttaattttagttcccctgagatcacaacttcctctccatcacccatcaattcgtctcaagcattaacaaacaaggtacaaatgacctctccgagcagcactggcagtcccatgtttaaattttcatctccaatcgtaaaatctactgaggcaaatgtactacctccatcatctattggatttacatttagtgtgcctgttgcaaaaacagcagaactttctggttctagtagtactttagaaccaattataagtagttcagctcatcatgtcactacagtgaacagtacaaattgtaagaagacaccacctgaagattgtgagggtccttttagacctgcagaaatcctgaaagaaggaagtgttctagatattctgaaaagccctggtttcgcatcgccgaagatagattctgttgctgctcagcccaccgcaacaagcccagtagtttatacaagaccagcaataagtagcttttcttctagtggaattgggtttggggagagtttaaaagctgggtcatcatggcagtgtgatacatgtctactccagaacaaagttacagacaacaaatgcatagcctgtcaagcagcaaaattgtcacccagagatactgctaaacagactggaattgaaacaccaaataaaagtggcaaaacaactctttctgcatcagggacaggctttggagacaaatttaaaccagtgataggcacttgggattgtgatacctgtttagtgcaaaataaacctgaagcaataaaatgtgtagcctgtgaaacaccgaaacctggaacttgtgtgaagcgagcccttacattgacagtggtttcggaaagtgctgagactatgactgcttcatcttccagctgcactgtaaccactggtaccttaggatttggagataaattcaaaaggcccattggatcttgggagtgttcagtatgctgtgtttctaataatgcagaagacaataagtgtgtgtcctgtatgtctgagaaaccaggaagttcagtacctgcttcaagtagcagcactgtacctgtctctctgccttctggaggctctctaggattggaaaagttcaagaaacccgagggaagctgggactgtgaattgtgcctagtgcagaataaggcagactctaccaaatgtttggcatgtgaaagtgcaaagccaggcacaaaatctgggtttaaaggctttgacacatcttcctcatcttcgaactcagcagcctcctcatccttcaaatttggtgtctcatcatcctcttctgggccttctcagactttaacaagcactggaaattttaaatttggagatcagggaggattcaaaataggtgtgtcatctgattctgggtctataaaccccatgagtgaaggctttaaattttctaaaccaataggagattttaaatttggagtttcatctgaatctaagcccgaagaagttaaaaaagatagtaagaatgataattttaagtttggactttcttctggtttaagcaacccagtttctttaactccatttcaatttggggtatctaatcttggacaggaagaaaagaaagaggaactgcccaaatcttcctctgcaggttttagctttggtacaggtgttattaactccacccctgctcctgctaacaccatagtgacctctgagaacaagagcagcttcaaccttggaaccatagaaaccaagagtgcttcagtggctcctttcacatgtaagacatcagaagctaaaaaagaagaaatgcctgccaccaaaggaggattctcttttggcaacgtggagcctgcctctctgccatctgcctcagtgtttgttttgggaaggacagaagagaaacagcaagagcctgtcacttctacttccctagtttttgggaagaaagctgacaatgaagagccaaagtgtcaaccagtgttttcctttgggaattcagagcaaaccaaagatgagaattcttcaaagtccacatttagttttagtatgacaaaaccatctgagaaggaatctgaacagccagcaaaagccacttttgcctttggagctcaaactagtactacagctgatcaaggtgcagcaaagccagtttttagtttcttgaacaacagttcctctagttcaagtacaccagccacttctgctggtggtggcatatttggtagttccacctcttcctccaatccacctgtggctacctttgtgtttggacagtccagcaatcctgtgagcagctctgcctttggtaacactgctgaatccagcacctctcagtctttgctattttctcaagatagcaaactagcaaccacatccagcacaggtacagctgtcaccccatttgtctttggtccaggagccagcagtaataatactaccacctctggtttcggctttggagccacaaccacatctagctctgcaggatcctcctttgtatttggaactggaccctcagcaccatctgccagtccagcatttggtgctaaccagaccccaacatttggacaaagtcaaggtgccagccagcccaatcccccaggctttggatctatatcatcttccacagcattatttcccactggttctcagcctgcaccacctacttttgggacagtgtcaagcagtagccagccccctgtgtttggacagcaacctagtcagtctgcatttggctctggaacaactcctaattctagttcggctttccagtttggcagcagcactacaaatttcaacttcacaaacaacagtccatcaggagtgttcacatttggtgcaaattctagcacacctgcagcctcagcccagccttcaggctcggggggctttccatttaaccagtctccagcagcatttacagtggggtcaaatgggaaaaatgtgttctcttcttctggaacttcattctctggtcgcaagataaagactgctgttagacgcaggaaataaaggtcacattggtgttgtactcaattttaacaacagctggtgccctgctttcagatactggattgtactttgtgctggggttatctgaagtcagatctgcctaaggacttctttaattttggaattttcctcctttctctttcgttacagaagccccaccctgcctcacccacccttttttaaataaataaatagctagactggtgactgattcttcagcaaaaatattttatgatccagcagattattcactgatttgacatagtctggctgtacccaggaatggagcctgcacggtgaatggctttgtatagaacctctttgtctacaccattatgtgcgctgataagcgttcatggaacgcgttgaaattgtaattatatctgaggaattctgtatagattagaattctgtatagattagagagtgttgaaacggatgatttctatgctgagtttgtgctggtgtatgtgtgaagtgagtgagttgggtgtattgtgcgctaaacttttctgatagaggaagcctgattaaagaatggtccgtgctaaggacttgttagatctagttcactctccatttaataattatatgctatttctatattttcattctcctatcacctgtcttgcctttttcattattttattatgaaacttgtgtaaatacaattttgtttctgtactttttggcataacataaatctgtgaacttgaaatttgaattttgtgttagagatttttttgttgtttgtttagtcttgtctcagattttattatgtaaatcccattattcaaagttgcctaaatccatttggaaatctttaaaaaaaaaattggggattcttaaagttgaatttattggcttttctgatccagttttgtttggaccaaaaaccagtattgtacaaagtattaagcatatatttttatatttactaaaatggactgtggtgactttggataataaggaaaagtttaatattaaagccatgtttattacagtataattaacatgttaaaccatgggataaatgccatcaataaaaaattatgacat

SEQ ID NO: 34 - protein, 1726 aa, Homo sapiens; identical to SEQ ID NO: 30
  ref <308>: DATABASE ACCESSION NUMBER: NP_624358
  ref <309>: DATABASE ENTRY DATE: 2005-04-23
  ref <313>: RELEVANT RESIDUES IN SEQ ID NO: (1)..(1726)

SEQ ID NO: 35 - DNA, 1644 bp, Artificial Sequence
  FLAG NUPA10(HD)
atggactacaaagacgatgacgataaagcaagaagagcatctgtgggatcctttaacaaatcatttggaacaccctttgggggtggcacaggtggctttggcacaacttcaacatttggacagaatactggctttggcactactagtggaggggcatttggaacatctgcatttggttctagcaacaatactggaggcctctttggaaattcacagactaaaccaggaggattgtttggaaccagttcatttagccagccagctacctccacaagcactggctttgggtttggtacgtcaacaggaacagcaaataccttgtttggaactgcaagcacagggaccagtctcttctcatcccaaaacaatgcctttgcacaaaataaaccaactggctttggcaattttggaaccagtactagcagtggaggactctttggaaccacaaataccacctctaatccttttggcagcacatctggctccctctttgggccaagtagttttacagctgctcctactgggactactattaaatttaaccctccaactggtacagatactatggtcaaagctggagttagcactaacataagtaccaagcaccagtgtattactgctatgaaagaatatgaaagcaagtcactagaggaacttcgtttagaggattatcaggctaacaggaagggcccacagaaccaggtgggagcaggtaccacaactggcttgtttgggtcttctccagccacttccagcgcaacaggactcttcagctcctccaccactaattcaggctttgcatatggtcagaacaaaactgcctttggaactagtacaactggatttggaacaaatccaggtggtctctttggccaacagaatcagcagactaccagcctcttcagcaaaccatttggccaggctacaaccacccagaacactggcttttcctttggtaataccagcaccataggacagccaagcaccaacaccatgggattatttggagtaacccaagcctcacagcctggaggtctttttgggacagctacaaacaccagcactgggacagcatttggaacaggaacaggtctctttgggcagaccaatactggatttggtgctgttggttcgaccctgtttggcaataacaagcttactacatttggaagcagcacaaccagtgcaccttcatttggtacaaccagtggcgggctctttggttttggcacaaataccagtgggaatagtatttttggaagtaaaccagcacctgggactcttggaactgggcttggtgcaggatttggaacagctcttggtgctggacaggcatctttgtttgggaacaaccaacctaagattggagggcctcttggtacaggagcctttggggcccctggatttaatactacgacagccactttgggctttggagccccccaggccccagtagaattcaagagtggtcggaagaagcgctgcccctacacgaagcaccagacactggagctggagaaggagtttctgttcaatatgtaccttactcgagagcggcgcctagagattagccgcagcgtccacctcacggacagacaagtgaaaatctggtttcagaaccgcaggatgaaactgaagaaaatg

SEQ ID NO: 36 - protein, 548 aa, Artificial Sequence
  FLAG NUPA10(HD)
MDYKDDDDKARRASVGSFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTAFGTGTGLFGQTNTGFGAVGSTLFGNNKLTTFGSSTTSAPSFGTTSGGLFGFGTNTSGNSIFGSKPAPGTLGTGLGAGFGTALGAGQASLFGNNQPKIGGPLGTGAFGAPGFNTTTATLGFGAPQAPVEFKSGRKKRCPYTKHQTLELEKEFLFNMYLTRERRLEISRSVHLTDRQVKIWFQNRRMKLKKM

SEQ ID NO: 37 - protein, 9 aa, Artificial Sequence
  Fragment of HOXD13, NA10 and NUPA10
RKKRRQRRR

SEQ ID NO: 38 - protein, 16 aa, Artificial Sequence
  Fragment of HOXB3 and HOXB4
RQIKIWFQNRRMKWKK